SEQUENCE LISTING (1 sequence)

SEQ ID NO: 1 - protein, 458 aa, Homo sapiens
  MISC_FEATURE (5)..(458): Amino Acid Sequence residues 13-466 of human Glucokinase
MKLMALTLVEQILAEFQLQEEDLKKVMRRMQKEMDRGLRLETHEEASVKMLPTYVRSTPEGSEVGDFLSLDLGGTNFRVMLVKVGEGEEGQWSVKTKHQMYSIPEDAMTGTAEMLFDYISECISDFLDKHQMKHKKLPLGFTFSFPVRHEDIDKGILLNWTKGFKASGAEGNNVVGLLRDAIKRRGDFEMDVVAMVNDTVATMISCYYEDHQCEVGMIVGTGCNACYMEEMQNVELVEGDEGRMCVNTEWGAFGDSGELDEFLLEYDRLVDESSANPGQQLYEKLIGGKYMGELVRLVLLRLVDENLLFHGEASEQLRTRGAFETRFVSQVESDTGDRKQIYNILSTLGLRPSTTDCDIVRRACESVSTRAAHMCSAGLAGVINRMRESRSEDVMRITVGVDGSVYKLHPSFKERFHASVRRLTPSCEITFIESEEGSGRGAALVSAVACKKACMLGQ

(19) United States
(12) Patent Application Publication
(10) Pub. No.: US 2007/0244169 A1
(43) Pub. Date: Oct. 18, 2007

(54) GLUCOKINASE ACTIVATORS

(76) Inventors: Jun Feng, Carlsbad, CA (US); Stephen L. Gwaltney, San Diego, CA (US); David J. Hosfield, Solana Beach, CA (US); Shigekazu Sasaki, Osaka (JP); Robert J. Skene, San Marcos, CA (US); Michael B. Wallace, San Diego, CA (US)

Correspondence Address: TAKEDA SAN DIEGO, INC., 10410 SCIENCE CENTER DRIVE, SAN DIEGO, CA, 92121, US

(21) Appl. No.: 11/613,059

(22) Filed: Dec. 19, 2006

Related U.S. Application Data
(60) Provisional application No. 60/752,750, filed on Dec. 20, 2005.

(51) Int. Cl. A61K 31/415; A61K 31/427; C07D 231/54; C07D 277/38

(52) U.S. Cl. 514/370; 514/407; 548/198; 548/356.5

(57) ABSTRACT

Compounds, pharmaceutical compositions, kits and methods are provided for use with glucokinase that comprise a compound selected from the group consisting of: 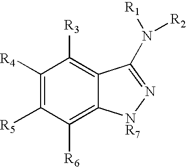 wherein the variables are as defined herein.

### RELATED APPLICATION

This application claims the benefit of U.S. Provisional Application No. 60/752,750, filed Dec. 20, 2005, which is incorporated herein by reference.

### FIELD OF THE INVENTION

The present invention relates to compounds that may be used to activate hexokinases, as well as compositions of matter, kits and articles of manufacture comprising these compounds. In addition, the present invention relates to methods of making said compounds, as well as intermediates useful in such methods. The invention also relates to methods for activating hexokinases and treatment methods using compounds according to the present invention. In particular, the present invention relates to glucokinase activators, compositions of matter and kits comprising these compounds and methods for activating glucokinase.

### BACKGROUND OF THE INVENTION

Glucokinase (GK, Hexokinase IV) is one of four hexokinases that are found in mammals (Colowick, S. P., in The Enzymes, Vol. 9 (P. Boyer, ed.) Academic Press, New York, N.Y., pages 1-48, 1973). The hexokinases catalyze the first step in the metabolism of glucose, i.e., the conversion of glucose to glucose-6-phosphate. Glucokinase is found principally in pancreatic β-cells and liver parenchymal cells, two cell types that are known to play critical roles in whole-body glucose homeostasis. Specifically, GK is a rate-controlling enzyme for glucose metabolism in these two cell types (Chipkin, S. R., Kelly, K. L., and Ruderman, N. B. in Joslin's Diabetes (C. R. Khan and G. C. Wier, eds.), Lea and Febiger, Philadelphia, Pa., pages 97-115, 1994).

The concentration of glucose at which GK demonstrates half-maximal activity is approximately 8 mM. The other three hexokinases are saturated with glucose at much lower concentrations (<1 mM). Therefore, the flux of glucose through the GK pathway rises as the concentration of glucose in the blood increases from fasting levels (5 mM) to postprandial levels following a carbohydrate-containing meal (about 10-15 mM) (Printz, R. G., Magnuson, M. A., and Granner, D. K. in Ann. Rev. Nutrition Vol. 13 (R. E. Olson, D. M. Bier, and D. B. McCormick, eds.), Annual Review, Inc., Palo Alto, Calif., pages 463-496, 1993). These findings suggest that GK functions as a glucose sensor in β-cells and hepatocytes (Meglasson, M. D. and Matschinsky, F. M. Amer. J. Physiol. 246, E1-E13, 1984).

More recently, studies in transgenic animals confirmed that GK does indeed play a critical role in whole-body glucose homeostasis. Animals that do not express GK die within days of birth with severe diabetes, while animals overexpressing GK have improved glucose tolerance (Grupe, A., Hultgren, B., Ryan, A. et al., Cell 83, 69-78, 1995; Ferrie, T., Riu, E., Bosch, F. et al., FASEB J., 10, 1213-1218, 1996). An increase in glucose exposure is coupled through GK in β-cells to increased insulin secretion and in hepatocytes to increased glycogen deposition and perhaps decreased glucose production.

The finding that type II maturity-onset diabetes of the young (MODY-2) is caused by loss of function mutations in the GK gene suggests that GK also functions as a glucose sensor in humans (Liang, Y., Kesavan, P., Wang, L. et al., Biochem. J. 309, 167-173, 1995). Additional evidence supporting an important role for GK in the regulation of glucose metabolism in humans was provided by the identification of patients that express a mutant form of GK with increased enzymatic activity. These patients exhibit a fasting hypoglycemia associated with an inappropriately elevated level of plasma insulin (Glaser, B., Kesavan, P., Heyman, M. et al., New England J. Med. 338, 226-230, 1998). Accordingly, compounds that activate GK and, thereby, increase the sensitivity of the GK sensor system are expected to be useful in the treatment of the hyperglycemia characteristic of all type II diabetes. Glucokinase activators should increase the flux of glucose metabolism in β-cells and hepatocytes, which will be coupled to increased insulin secretion.

There is a continued need to find new therapeutic agents to treat human diseases. The hexokinases, specifically but not limited to glucokinase, are especially attractive targets for the discovery of new therapeutics due to their important role in diabetes, hyperglycemia and other diseases.

### SUMMARY OF THE INVENTION

The present invention relates to compounds that activate glucokinase. The present invention also provides compositions, articles of manufacture and kits comprising these compounds.

In one embodiment, a pharmaceutical composition is provided that comprises a glucokinase activator according to the present invention as an active ingredient. Pharmaceutical compositions according to the invention may optionally comprise 0.001%-100% of one or more activators of this invention. These pharmaceutical compositions may be administered or coadministered by a wide variety of routes, including for example, orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compositions may also be administered or coadministered in slow release dosage forms.

The invention is also directed to kits and other articles of manufacture for treating disease states associated with glucokinase.

In one embodiment, a kit is provided that comprises a composition comprising at least one glucokinase activator of the present invention in combination with instructions. The instructions may indicate the disease state for which the composition is to be administered, storage information, dosing information and/or instructions regarding how to administer the composition. The kit may also comprise packaging materials. The packaging material may comprise a container for housing the composition. The kit may also optionally comprise additional components, such as syringes for administration of the composition. The kit may comprise the composition in single or multiple dose forms.

In another embodiment, an article of manufacture is provided that comprises a composition comprising at least one glucokinase activator of the present invention in combination with packaging materials. The packaging material may comprise a container for housing the composition. The container may optionally comprise a label indicating the disease state for which the composition is to be administered, storage information, dosing information and/or instructions regarding how to administer the composition. The kit may also optionally comprise additional components, such as syringes for administration of the composition. The kit may comprise the composition in single or multiple dose forms.

Also provided are methods for preparing compounds, compositions and kits according to the present invention. For example, several synthetic schemes are provided herein for synthesizing compounds according to the present invention.

Also provided are methods for using compounds, compositions, kits and articles of manufacture according to the present invention.

In one embodiment, the compounds, compositions, kits and articles of manufacture are used to modulate glucokinase. In particular, the compounds, compositions, kits and articles of manufacture can be used to activate glucokinase.

In another embodiment, the compounds, compositions, kits and articles of manufacture are used to treat a disease state for which increasing glucokinase activity ameliorates the pathology and/or symptomology of the disease state.

In another embodiment, a compound is administered to a subject wherein glucokinase activity within the subject is altered and, in one embodiment, increased.

In another embodiment, a prodrug of a compound is administered to a subject that is converted to the compound in vivo where it activates glucokinase.

In another embodiment, a method of activating glucokinase is provided that comprises contacting glucokinase with a compound according to the present invention.

In another embodiment, a method of activating glucokinase is provided that comprises causing a compound according to the present invention to be present in a subject in order to activate glucokinase in vivo.

In another embodiment, a method of activating glucokinase is provided that comprises administering a first compound to a subject that is converted in vivo to a second compound wherein the second compound activates glucokinase in vivo. It is noted that the compounds of the present invention may be the first or second compounds.

In another embodiment, a therapeutic method is provided that comprises administering a compound according to the present invention.

In another embodiment, a method of treating a condition in a patient that is known to be mediated by glucokinase, or which is known to be treated by glucokinase activators, is provided comprising administering to the patient a therapeutically effective amount of a compound according to the present invention.

In another embodiment, a method is provided for treating a disease state for which increasing glucokinase activity ameliorates the pathology and/or symptomology of the disease state, the method comprising: causing a compound according to the present invention to be present in a subject in a therapeutically effective amount for the disease state.

In another embodiment, a method is provided for treating a disease state for which increasing glucokinase activity ameliorates the pathology and/or symptomology of the disease state, the method comprising: administering a first compound to a subject that is converted in vivo to a second compound such that the second compound is present in the subject in a therapeutically effective amount for the disease state. It is noted that the compounds of the present invention may be the first or second compounds.

In another embodiment, a method is provided for treating a disease state for which increasing glucokinase activity ameliorates the pathology and/or symptomology of the disease state, the method comprising: administering a compound according to the present invention to a subject such that the compound is present in the subject in a therapeutically effective amount for the disease state.

In another embodiment, a method is provided for using a compound according to the present invention in order to manufacture a medicament for use in the treatment of a disease state that is known to be mediated by glucokinase, or that is known to be treated by glucokinase activators.

It is noted in regard to all of the above embodiments that the present invention is intended to encompass all pharmaceutically acceptable ionized forms (e.g., salts) and solvates (e.g., hydrates) of the compounds, regardless of whether such ionized forms and solvates are specified since it is well know in the art to administer pharmaceutical agents in an ionized or solvated form. It is also noted that unless a particular stereochemistry is specified, recitation of a compound is intended to encompass all possible stereoisomers (e.g., enantiomers or diastereomers depending on the number of chiral centers), independent of whether the compound is present as an individual isomer or a mixture of isomers. Further, unless otherwise specified, recitation of a compound is intended to encompass all possible resonance forms and tautomers. With regard to the claims, the language “compound comprising the formula” is intended to encompass the compound and all pharmaceutically acceptable ionized forms and solvates, all possible stereoisomers, and all possible resonance forms and tautomers unless otherwise specifically specified in the particular claim.

It is further noted that prodrugs may also be administered which are altered in vivo and become a compound according to the present invention. The various methods of using the compounds of the present invention are intended, regardless of whether prodrug delivery is specified, to encompass the administration of a prodrug that is converted in vivo to a compound according to the present invention. It is also noted that certain compounds of the present invention may be altered in vivo prior to activating glucokinase and thus may themselves be prodrugs for another compound. Such prodrugs of another compound may or may not themselves independently have glucokinase activity.

### BRIEF DESCRIPTION OF THE FIGURES

FIG. 1 illustrates SEQ. ID No. 1 referred to in this application.

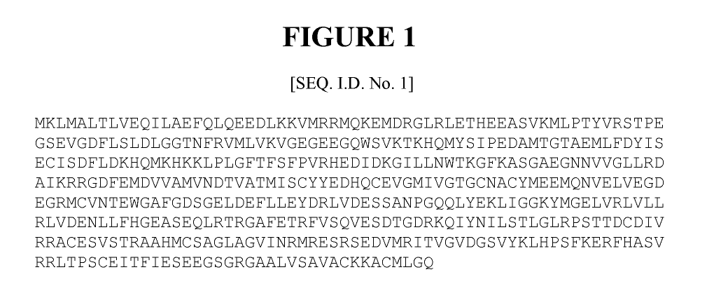

### DEFINITIONS

Unless otherwise stated, the following terms used in the specification and claims shall have the following meanings for the purposes of this application.

“Alicyclic” means a moiety comprising a non-aromatic ring structure. Alicyclic moieties may be saturated or partially unsaturated with one, two or more double or triple bonds. Alicyclic moieties may also optionally comprise heteroatoms such as nitrogen, oxygen and sulfur. The nitrogen atoms can be optionally quaternerized or oxidized and the sulfur atoms can be optionally oxidized. Examples of alicyclic moieties include, but are not limited to moieties with (C₃₋₈) rings such as cyclopropyl, cyclohexane, cyclopentane, cyclopentene, cyclopentadiene, cyclohexane, cyclohexene, cyclohexadiene, cycloheptane, cycloheptene, cycloheptadiene, cyclooctane, cyclooctene, and cyclooctadiene.

“Aliphatic” means a moiety characterized by a straight or branched chain arrangement of constituent carbon atoms and may be saturated or partially unsaturated with one, two or more double or triple bonds.

“Alkenyl” means a straight or branched, carbon chain that contains at least one carbon-carbon double bond (—CR═CR′— or —CR═CR′R″, wherein R, R′ and R″ are each independently hydrogen or further substituents). Examples of alkenyl include vinyl, allyl, isopropenyl, pentenyl, hexenyl, heptenyl, 1-propenyl, 2-butenyl, 2-methyl-2-butenyl, and the like. In particular embodiments, “alkenyl,” either alone or represented along with another radical, can be a (C₂₋₂₀)alkenyl, a (C₂₋₁₅)alkenyl, a (C₂₋₁₀)alkenyl, a (C₂₋₅)alkenyl or a (C₂₋₃)alkenyl. Alternatively, “alkenyl,” either alone or represented along with another radical, can be a (C₂)alkenyl, a (C₃)alkenyl or a (C₄)alkenyl.

“Alkenylene” means a straight or branched, divalent carbon chain having one or more carbon-carbon double bonds (—CR═CR′—, wherein R and R′ are each independently hydrogen or further substituents). Examples of alkenylene include ethene-1,2-diyl, propene-1,3-diyl, methylene-1,1-diyl, and the like. In particular embodiments, “alkenylene,” either alone or represented along with another radical, can be a (C₂₋₂₀) alkenylene, a (C₂₋₁₅) alkenylene, a (C₂₋₁₀) alkenylene, a (C₂₋₅) alkenylene or a (C₂₋₃) alkenylene. Alternatively, “alkenylene,” either alone or represented along with another radical, can be a (C₂) alkenylene, a (C₃) alkenylene or a (C₄) alkenylene.

“Alkoxy” means an oxygen moiety having a further alkyl substituent. The alkoxy groups of the present invention can be optionally substituted.

“Alkyl” represented by itself means a straight or branched, saturated or unsaturated, aliphatic radical having a chain of carbon atoms, optionally with one or more of the carbon atoms being replaced with oxygen (See “oxaalkyl”), a carbonyl group (See “oxoalkyl), sulfur (See “thioalkyl”), and/or nitrogen (See “azaalkyl”). (C_{X})alkyl and (C_{X-Y})alkyl are typically used where X and Y indicate the number of carbon atoms in the chain. For example, (C₁₋₆)alkyl includes alkyls that have a chain of between 1 and 6 carbons (e.g., methyl, ethyl, propyl, isopropyl, butyl, sec-butyl, isobutyl, tert-butyl, vinyl, allyl, 1-propenyl, isopropenyl, 1-butenyl, 2-butenyl, 3-butenyl, 2-methylallyl, ethynyl, 1-propynyl, 2-propynyl, and the like). Alkyl represented along with another radical (e.g., as in arylalkyl, heteroarylalkyl and the like) means a straight or branched, saturated or unsaturated aliphatic divalent radical having the number of atoms indicated or when no atoms are indicated means a bond (e.g., (C₆₋₁₀)aryl(C₁₋₃)alkyl includes, benzyl, phenethyl, 1-phenylethyl, 3-phenylpropyl, 2-thienylmethyl, 2-pyridinylmethyl and the like). In particular embodiments, “alkyl,” either alone or represented along with another radical, can be a (C₁₋₂₀)alkyl, a (C₁₋₁₅)alkyl, a (C₁₋₁₀)alkyl, a (C₁₋₅)alkyl or a (C₁₋₃)alkyl. Alternatively, “alkyl,” either alone or represented along with another radical, can be a (C₁)alkyl, a (C₂)alkyl or a (C₃)alkyl.

“Alkylene”, unless indicated otherwise, means a straight or branched, saturated or unsaturated, aliphatic, divalent radical. (C_{X})alkylene and (C_{X-Y})alkylene are typically used where X and Y indicate the number of carbon atoms in the chain. For example, (C₁₋₆)alkylene includes methylene (—CH₂—), ethylene (—CH₂CH₂—), trimethylene (—CH₂CH₂CH₂—), tetramethylene (—CH₂CH₂CH₂CH₂—) 2-butenylene (—CH₂CH═CHCH₂—), 2-methyltetramethylene (—CH₂CH(CH₃)CH₂CH₂—), pentamethylene (—CH₂CH₂CH₂CH₂CH₂—) and the like. In particular embodiments, “alkylene,” either alone or represented along with another radical, can be a (C₁₋₂₀)alkylene, a (C₁₋₁₅)alkylene, a (C₁₋₁₀)alkylene, a (C₁₋₅)alkylene or a (C₁₋₃)alkylene. Alternatively, “alkylene,” either alone or represented along with another radical, can be a (C₁)alkylene, a (C₂)alkylene or a (C₃)alkylene.

“Alkylidene” means a straight or branched, saturated or unsaturated, aliphatic radical connected to the parent molecule by a double bond. (C_{X})alkylidene and (C_{X-Y})alkylidene are typically used where X and Y indicate the number of carbon atoms in the chain. For example, (C₁₋₆)alkylidene includes methylene (═CH₂), ethylidene (═CHCH₃), isopropylidene (═C(CH₃)₂), propylidene (═CHCH₂CH₃), allylidene (═CH—CH═CH₂), and the like. In particular embodiments, “alkylidene,” either alone or represented along with another radical, can be a (C₁₋₂₀)alkylidene, a (C₁₋₁₅)alkylidene, a (C₁₋₁₀)alkylidene, a (C₁₋₅)alkylidene or a (C₁₋₃)alkylidene. Alternatively, “alkylidene,” either alone or represented along with another radical, can be a (C₁)alkylidene, a (C₂)alkylidene or a (C₃)alkylidene.

“Alkynyl” means a straight or branched, carbon chain that contains at least one carbon-carbon triple bond (—C≡C— or —C≡CR, wherein R is hydrogen or a further substituent). Examples of alkynyl include ethynyl, propargyl, 3-methyl-1-pentynyl, 2-heptynyl and the like. In particular embodiments, “alkynyl,” either alone or represented along with another radical, can be a (C₂₋₂₀)alkynyl, a (C₂₋₁₅)alkynyl, a (C₂₋₁₀)alkynyl, a (C₂₋₅)alkynyl or a (C₂₋₃)alkynyl. Alternatively, “alkynyl,” either alone or represented along with another radical, can be a (C₂)alkynyl, a (C₃)alkynyl or a (C₄)alkynyl.

“Alkynylene” means a straight or branched, divalent carbon chain having one or more carbon-carbon triple bonds (—CR≡CR′—, wherein R and R′ are each independently hydrogen or further substituents). Examples of alkynylene include ethyne-1,2-diyl, propyne-1,3-diyl, and the like. In particular embodiments, “alkynylene,” either alone or represented along with another radical, can be a (C₂₋₂₀) alkynylene, a (C₂₋₁₅) alkynylene, a (C₂₋₁₀) alkynylene, a (C₂₋₅) alkynylene or a (C₂₋₃) alkynylene. Alternatively, “alkenylene,” either alone or represented along with another radical, can be a (C₂) alkynylene, a (C₃) alkynylene or a (C₄) alkynylene.

“Amino” means a nitrogen moiety having two further substituents where, for example, a hydrogen or carbon atom is attached to the nitrogen. For example, representative amino groups include —NH₂, —NHCH₃, —N(CH₃)₂, —NH((C₁₋₁₀)alkyl), —N((C₁₋₁₀)alkyl)₂, —NH(aryl), —NH(heteroaryl), —N(aryl)₂, —N(heteroaryl)₂, and the like. Optionally, the two substituents together with the nitrogen may also form a ring. Unless indicated otherwise, the compounds of the invention containing amino moieties may include protected derivatives thereof. Suitable protecting groups for amino moieties include acetyl, tert-butoxycarbonyl, benzyloxycarbonyl, and the like.

“Animal” includes humans, non-human mammals (e.g., dogs, cats, rabbits, cattle, horses, sheep, goats, swine, deer, and the like) and non-mammals (e.g., birds, and the like).

“Aromatic” means a moiety wherein the constituent atoms make up an unsaturated ring system, all atoms in the ring system are sp² hybridized and the total number of pi electrons is equal to 4n+2. An aromatic ring may be such that the ring atoms are only carbon atoms or may include carbon and non-carbon atoms (See “heteroaryl”).

“Aryl” means a monocyclic or polycyclic ring assembly wherein each ring is aromatic or when fused with one or more rings forms an aromatic ring assembly. If one or more ring atoms is not carbon (e.g., N, S), the aryl is a heteroaryl. (C_{X})aryl and (C_{X-Y})aryl are typically used where X and Y indicate the number of carbon atoms in the ring. In particular embodiments, “aryl,” either alone or represented along with another radical, can be a (C₃₋₁₄)aryl, a (C₃₋₁₀)aryl, a (C₃₋₇)aryl, a (C₈₋₁₀)aryl or a (C₅₋₇)aryl. Alternatively, “aryl,” either alone or represented along with another radical, can be a (C₅)aryl, a (C₆)aryl, a (C₇)aryl, a (C₈)aryl, a (C₉)aryl or a (C₁₀)aryl.

“Azaalkyl” means an alkyl, as defined above, except where one or more of the carbon atoms forming the alkyl chain are replaced with substituted or unsubstituted nitrogen atoms (—NR— or —NRR′, wherein R and R′ are each independently hydrogen or further substituents). For example, a (C₁₋₁₀)azaalkyl refers to a chain comprising between 1 and 10 carbons and one or more nitrogen atoms.

“Bicycloalkyl” means a saturated or partially unsaturated fused, spiro or bridged bicyclic ring assembly. In particular embodiments, “bicycloalkyl,” either alone or represented along with another radical, can be a (C₄₋₁₅)bicycloalkyl, a (C₄₋₁₀)bicycloalkyl, a (C₆₋₁₀)bicycloalkyl or a (C₈₋₁₀)bicycloalkyl. Alternatively, “bicycloalkyl,” either alone or represented along with another radical, can be a (C₈)bicycloalkyl, a (C₉)bicycloalkyl or a (C₁₀)bicycloalkyl.

“Bicycloaryl” means a fused, spiro or bridged bicyclic ring assembly wherein at least one of the rings comprising the assembly is aromatic. (C_{X})bicycloaryl and (C_{X-Y})bicycloaryl are typically used where X and Y indicate the number of carbon atoms in the bicyclic ring assembly and directly attached to the ring. In particular embodiments, “bicycloaryl,” either alone or represented along with another radical, can be a (a (C₄₋₁₅)bicycloaryl, a (C₄₋₁₀)bicycloaryl, a (C₆₋₁₀)bicycloaryl or a (C₈₋₁₀)bicycloaryl. Alternatively, “bicycloalkyl,” either alone or represented along with another radical, can be a (C₈)bicycloaryl, a (C₉)bicycloaryl or a (C₁₀)bicycloaryl.

“Bridging ring” and “bridged ring” as used herein refer to a ring that is bonded to another ring to form a compound having a bicyclic or polycyclic structure where two ring atoms that are common to both rings are not directly bound to each other. Non-exclusive examples of common compounds having a bridging ring include borneol, norbornane, 7-oxabicyclo[2.2.1]heptane, and the like. One or both rings of the bicyclic system may also comprise heteroatoms.

“Carbamoyl” means the radical —OC(O)NRR′, wherein R and R′ are each independently hydrogen or further substituents.

“Carbocycle” means a ring consisting of carbon atoms.

“Carbonyl” means the radical —C(═O)— and/or —C(═O)R, wherein R is hydrogen or a further substituent. It is noted that the carbonyl radical may be further substituted with a variety of substituents to form different carbonyl groups including acids, acid halides, aldehydes, amides, esters, and ketones.

“Carboxy” means the radical —C(═O)—O— and/or —C(═O)—OR, wherein R is hydrogen or a further substituent. It is noted that compounds of the invention containing carboxy moieties may include protected derivatives thereof, i.e., where the oxygen is substituted with a protecting group. Suitable protecting groups for carboxy moieties include benzyl, tert-butyl, and the like.

“Cyano” means the radical —CN.

“Cycloalkyl” means a non-aromatic, saturated or partially unsaturated, monocyclic, bicyclic or polycyclic ring assembly. (C_{X})cycloalkyl and (C_{X-Y})cycloalkyl are typically used where X and Y indicate the number of carbon atoms in the ring assembly. For example, (C₃₋₁₀)cycloalkyl includes cyclopropyl, cyclobutyl, cyclopentyl, cyclohexyl, cyclohexenyl, 2,5-cyclohexadienyl, bicyclo[2.2.2]octyl, adamantan-1-yl, decahydronaphthyl, oxocyclohexyl, dioxocyclohexyl, thiocyclohexyl, 2-oxobicyclo[2.2.1]hept-1-yl, and the like. In particular embodiments, “cycloalkyl,” either alone or represented along with another radical, can be a (C₃₋₁₄)cycloalkyl, a (C₃₋₁₀)cycloalkyl, a (C₃₋₇)cycloalkyl, a (C₈₋₁₀)cycloalkyl or a (C₅₋₇)cycloalkyl. Alternatively, “cycloalkyl,” either alone or represented along with another radical, can be a (C₅)cycloalkyl, a (C₆)cycloalkyl, a (C₇)cycloalkyl, a (C₈)cycloalkyl, a (C₉)cycloalkyl or a (C₁₀)cycloalkyl.

“Cycloalkylene” means a divalent, saturated or partially unsaturated, monocyclic, bicyclic or polycyclic ring assembly. (C_{X})cycloalkylene and (C_{X-Y})cycloalkylene are typically used where X and Y indicate the number of carbon atoms in the ring assembly. In particular embodiments, “cycloalkylene,” either alone or represented along with another radical, can be a (C₃₋₁₄)cycloalkylene, a (C₃₋₁₀)cycloalkylene, a (C₃₋₇)cycloalkylene, a (C₈₋₁₀)cycloalkylene or a (C₅₋₇)cycloalkylene. Alternatively, “cycloalkylene,” either alone or represented along with another radical, can be a (C₅)cycloalkylene, a (C₆)cycloalkylene, a (C₇)cycloalkylene, a (C₈)cycloalkylene., a (C₉)cycloalkylene or a (C₁₀)cycloalkylene.

“Disease” specifically includes any unhealthy condition of an animal or part thereof and includes an unhealthy condition that may be caused by, or incident to, medical or veterinary therapy applied to that animal, i.e., the “side effects” of such therapy.

“Fused ring” as used herein refers to a ring that is bonded to another ring to form a compound having a bicyclic structure when the ring atoms that are common to both rings are directly bound to each other. Non-exclusive examples of common fused rings include decalin, naphthalene, anthracene, phenanthrene, indole, furan, benzofuran, quinoline, and the like. Compounds having fused ring systems may be saturated, partially saturated, carbocyclics, heterocyclics, aromatics, heteroaromatics, and the like.

“Halo” means fluoro, chloro, bromo or iodo.

“Heteroalkyl” means alkyl, as defined in this application, provided that one or more of the atoms within the alkyl chain is a heteroatom. In particular embodiments, “heteroalkyl,” either alone or represented along with another radical, can be a hetero(C₁₋₂₀)alkyl, a hetero(C₁₋₅)alkyl, a hetero(C₁₋₁₀)alkyl, a hetero(C₁₋₅)alkyl, a hetero(C₁₋₃)alkyl or a hetero(C₁₋₂)alkyl. Alternatively, “heteroalkyl,” either alone or represented along with another radical, can be a hetero(C₁)alkyl, a hetero(C₂)alkyl or a hetero(C₃)alkyl.

“Heteroaryl” means a monocyclic, bicyclic or polycyclic aromatic group wherein at least one ring atom is a heteroatom and the remaining ring atoms are carbon. Monocyclic heteroaryl groups include, but are not limited to, cyclic aromatic groups having five or six ring atoms, wherein at least one ring atom is a heteroatom and the remaining ring atoms are carbon. The nitrogen atoms can be optionally quaternerized and the sulfur atoms can be optionally oxidized. Heteroaryl groups of this invention include, but are not limited to, those derived from furan, imidazole, isothiazole, isoxazole, oxadiazole, oxazole, 1,2,3-oxadiazole, pyrazine, pyrazole, pyridazine, pyridine, pyrimidine, pyrroline, thiazole, 1,3,4-thiadiazole, triazole and tetrazole. “Heteroaryl” also includes, but is not limited to, bicyclic or tricyclic rings, wherein the heteroaryl ring is fused to one or two rings independently selected from the group consisting of an aryl ring, a cycloalkyl ring, a cycloalkenyl ring, and another monocyclic heteroaryl or heterocycloalkyl ring. These bicyclic or tricyclic heteroaryls include, but are not limited to, those derived from benzo[b]furan, benzo[b]thiophene, benzimidazole, imidazo[4,5-c]pyridine, quinazoline, thieno[2,3-c]pyridine, thieno[3,2-b]pyridine, thieno[2,3-b]pyridine, indolizine, imidazo[1,2a]pyridine, quinoline, isoquinoline, phthalazine, quinoxaline, naphthyridine, quinolizine, indole, isoindole, indazole, indoline, benzoxazole, benzopyrazole, benzothiazole, imidazo[1,5-a]pyridine, pyrazolo[1,5-a]pyridine, imidazo[1,2-a]pyrimidine, imidazo[1,2-c]pyrimidine, imidazo[1,5-a]pyrimidine, imidazo[1,5-c]pyrimidine, pyrrolo[2,3-b]pyridine, pyrrolo[2,3-c]pyridine, pyrrolo[3,2-c]pyridine, pyrrolo[3,2-b]pyridine, pyrrolo[2,3-d]pyrimidine, pyrrolo[3,2-d]pyrimidine, pyrrolo[2,3-b]pyrazine, pyrazolo[1,5-a]pyridine, pyrrolo[1,2-b]pyridazine, pyrrolo[1,2-c]pyrimidine, pyrrolo[1,2-a]pyrimidine, pyrrolo[1,2-a]pyrazine, triazo[1,5-a]pyridine, pteridine, purine, carbazole, acridine, phenazine, phenothiazene, phenoxazine, 1,2-dihydropyrrolo[3,2,1-hi]indole, indolizine, pyrido[1,2-a]indole and 2(1H)-pyridinone. The bicyclic or tricyclic heteroaryl rings can be attached to the parent molecule through either the heteroaryl group itself or the aryl, cycloalkyl, cycloalkenyl or heterocycloalkyl group to which it is fused. The heteroaryl groups of this invention can be substituted or unsubstituted. In particular embodiments, “heteroaryl,” either alone or represented along with another radical, can be a hetero(C₁₋₁₃)aryl, a hetero(C₂₋₁₃)aryl, a hetero(C₂₋₆)aryl, a hetero(C₃₋₉)aryl or a hetero(C₅₋₉)aryl. Alternatively, “heteroaryl,” either alone or represented along with another radical, can be a hetero(C₃)aryl, a hetero(C₄)aryl, a hetero(C₅)aryl, a hetero(C₆)aryl, a hetero(C₇)aryl, a hetero(C₈)aryl or a hetero(C₉)aryl.

“Heteroatom” refers to an atom that is not a carbon atom. Particular examples of heteroatoms include, but are not limited to, nitrogen, oxygen, and sulfur.

“Heteroatom moiety” includes a moiety where the atom by which the moiety is attached is not a carbon. Examples of heteroatom moieties include —NR—, —N⁺(O⁻)═, —O—, —S— or —S(O)₂—, wherein R is hydrogen or a further substituent.

“Heterobicycloalkyl” means bicycloalkyl, as defined in this application, provided that one or more of the atoms within the ring is a heteroatom. For example hetero(C₉₋₁₂)bicycloalkyl as used in this application includes, but is not limited to, 3-aza-bicyclo[4.1.0]hept-3-yl, 2-aza-bicyclo[3.1.0]hex-2-yl, 3-aza-bicyclo[3.1.0]hex-3-yl, and the like. In particular embodiments, “heterobicycloalkyl,” either alone or represented along with another radical, can be a hetero(C₁₋₁₄)bicycloalkyl, a hetero(C₄₋₁₄)bicycloalkyl, a hetero(C₄₋₉)bicycloalkyl or a hetero(C₅₋₉)bicycloalkyl. Alternatively, “heterobicycloalkyl,” either alone or represented along with another radical, can be a hetero(C₅)bicycloalkyl, hetero(C₆)bicycloalkyl, hetero(C₇)bicycloalkyl, hetero(C₈)bicycloalkyl or a hetero(C₉)bicycloalkyl.

“Heterobicycloaryl” means bicycloaryl, as defined in this application, provided that one or more of the atoms within the ring is a heteroatom. For example, hetero(C₄₋₁₂)bicycloaryl as used in this application includes, but is not limited to, 2-amino-4-oxo-3,4-dihydropteridin-6-yl, tetrahydroisoquinolinyl, and the like. In particular embodiments, “heterobicycloaryl,” either alone or represented along with another radical, can be a hetero(C₁₋₁₄)bicycloaryl, a hetero(C₄₋₁₄)bicycloaryl, a hetero(C₄₋₉)bicycloarylor a hetero(C₅₋₉)bicycloaryl. Alternatively, “heterobicycloaryl,” either alone or represented along with another radical, can be a hetero(C₅)bicycloaryl, hetero(C₆)bicycloaryl, hetero(C₇)bicycloaryl, hetero(C₈)bicycloaryl or a hetero(C₉)bicycloaryl.

“Heterocycloalkyl” means cycloalkyl, as defined in this application, provided that one or more of the atoms forming the ring is a heteroatom selected, independently from N, O, or S. Non-exclusive examples of heterocycloalkyl include piperidyl, 4-morpholyl, 4-piperazinyl, pyrrolidinyl, perhydropyrrolizinyl, 1,4-diazaperhydroepinyl, 1,3-dioxanyl, 1,4-dioxanyl and the like. In particular embodiments, “heterocycloalkyl,” either alone or represented along with another radical, can be a hetero(C₁₋₁₃)cycloalkyl, a hetero(C₁₋₉)cycloalkyl, a hetero(C₁₋₆)cycloalkyl, a hetero(C₅₋₉)cycloalkyl or a hetero(C₂₋₆)cycloalkyl. Alternatively, “heterocycloalkyl,” either alone or represented along with another radical, can be a hetero(C₂)cycloalkyl, a hetero(C₃)cycloalkyl, a hetero(C₄)cycloalkyl, a hetero(C₅)cycloalkyl, a hetero(C₆)cycloalkyl, hetero(C₇)cycloalkyl, hetero(C₈)cycloalkyl or a hetero(C₉)cycloalkyl.

“Heterocycloalkylene” means cycloalkylene, as defined in this application, provided that one or more of the ring member carbon atoms is replaced by a heteroatom. In particular embodiments, “heterocycloalkylene,” either alone or represented along with another radical, can be a hetero(C₁₋₁₃)cycloalkylene, a hetero(C₁₋₉)cycloalkylene, a hetero(C₁₋₆)cycloalkylene, a hetero(C₅₋₉)cycloalkylene or a hetero(C₂₋₆)cycloalkylene. Alternatively, “heterocycloalkylene,” either alone or represented along with another radical, can be a hetero(C₂)cycloalkylene, a hetero(C₃)cycloalkylene, a hetero(C₄)cycloalkylene, a hetero(C₅)cycloalkylene, a hetero(C₆)cycloalkylene, hetero(C₇)cycloalkylene, hetero(C₈)cycloalkylene or a hetero(C₉)cycloalkylene.

“Hydroxy” means the radical —OH.

“IC₅₀” means the molar concentration of an inhibitor that produces 50% inhibition of the target enzyme.

“Isomers” means compounds having identical molecular formulae but differing in the nature or sequence of bonding of their atoms or in the arrangement of their atoms in space. Isomers that differ in the arrangement of their atoms in space are termed “stereoisomers.” Stereoisomers that are not mirror images of one another are termed “diastereomers” and stereoisomers that are nonsuperimposable mirror images are termed “enantiomers” or sometimes “optical isomers.” A carbon atom bonded to four nonidentical substituents is termed a “chiral center.” A compound with one chiral center has two enantiomeric forms of opposite chirality. A mixture of the two enantiomeric forms is termed a “racemic mixture.” A compound that has more than one chiral center has 2ⁿ⁻¹ enantiomeric pairs, where n is the number of chiral centers. Compounds with more than one chiral center may exist as ether an individual diastereomer or as a mixture of diastereomers, termed a “diastereomeric mixture.” When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R- and S-sequencing rules of Cahn, Ingold and Prelog. Conventions for stereochemical nomenclature, methods for the determination of stereochemistry and the separation of stereoisomers are well known in the art (e.g., see “Advanced Organic Chemistry”, 4th edition, March, Jerry, John Wiley & Sons, New York, 1992).

“Leaving group” means the group with the meaning conventionally associated with it in synthetic organic chemistry, i.e., an atom or group displaceable under reaction (e.g., alkylating) conditions. Examples of leaving groups include, but are not limited to, halo (e.g., F, Cl, Br and I), alkyl (e.g., methyl and ethyl) and sulfonyloxy (e.g., mesyloxy, ethanesulfonyloxy, benzenesulfonyloxy and tosyloxy), thiomethyl, thienyloxy, dihalophosphinoyloxy, tetrahalophosphoxy, benzyloxy, isopropyloxy, acyloxy, and the like.

“Nitro” means the radical —NO₂.

“Oxaalkyl” means an alkyl, as defined above, except where one or more of the carbon atoms forming the alkyl chain are replaced with oxygen atoms (—O— or —OR, wherein R is hydrogen or a further substituent). For example, an oxa(C₁₋₁₀)alkyl refers to a chain comprising between 1 and 10 carbons and one or more oxygen atoms.

“Oxoalkyl” means an alkyl, as defined above, except where one or more of the carbon atoms forming the alkyl chain are replaced with carbonyl groups (—C(═O)— or —C(═O)—R, wherein R is hydrogen or a further substituent). The carbonyl group may be an aldehyde, ketone, ester, amide, acid or acid halide. For example, an oxo(C₁₋₁₀)alkyl refers to a chain comprising between 1 and 10 carbon atoms and one or more carbonyl groups.

“Oxy” means the radical —O—. It is noted that the oxy radical may be further substituted with a variety of substituents to form different oxy groups including hydroxy, alkoxy, aryloxy, heteroaryloxy or carbonyloxy.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like.

Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

“Polycyclic ring” includes bicyclic and multi-cyclic rings. The individual rings comprising the polycyclic ring can be fused, spiro or bridging rings.

“Prodrug” means a compound that is convertible in vivo metabolically into an activator according to the present invention. The prodrug itself may or may not also have activity with respect to a given target protein. For example, a compound comprising a hydroxy group may be administered as an ester that is converted by hydrolysis in vivo to the hydroxy compound. Suitable esters that may be converted in vivo into hydroxy compounds include acetates, citrates, lactates, tartrates, malonates, oxalates, salicylates, propionates, succinates, fumarates, maleates, methylene-bis-b-hydroxynaphthoates, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, a compound comprising an amine group may be administered as an amide that is converted by hydrolysis in vivo to the amine compound.

“Protected derivatives” means derivatives of activators in which a reactive site or sites are blocked with protecting groups. Protected derivatives are useful in the preparation of activators or in themselves may be active as activators. A comprehensive list of suitable protecting groups can be found in T. W. Greene, Protecting Groups in Organic Synthesis, 3rd edition, John Wiley & Sons, Inc. 1999.

“Ring” and “ring assembly” means a carbocyclic or a heterocyclic system. The system can be monocyclic, bicyclic or polycyclic. In addition, for bicyclic and polycyclic systems, the individual rings comprising the polycyclic ring can be fused, spiro or bridging rings.

“Subject” includes humans, non-human mammals (e.g., dogs, cats, rabbits, cattle, horses, sheep, goats, swine, deer, and the like) and non-mammals (e.g., birds, and the like).

“Substituent convertible to hydrogen in vivo” means any group that is convertible to a hydrogen atom by enzymological or chemical means including, but not limited to, hydrolysis and hydrogenolysis. Examples include hydrolyzable groups, such as acyl groups, groups having an oxycarbonyl group, amino acid residues, peptide residues, o-nitrophenylsulfenyl, trimethylsilyl, tetrahydro-pyranyl, diphenylphosphinyl, and the like. Examples of acyl groups include formyl, acetyl, trifluoroacetyl, and the like. Examples of groups having an oxycarbonyl group include ethoxycarbonyl, t-butoxycarbonyl [(CH₃)₃C—OCO—], benzyloxycarbonyl, p-methoxybenzyloxycarbonyl, vinyloxycarbonyl, β-(p-toluenesulfonyl)ethoxycarbonyl, and the like. Examples of suitable amino acid residues include amino acid residues per se and amino acid residues that are protected with a protecting group. Suitable amino acid residues include, but are mnot limited to, residues of Gly (glycine), Ala (alanine; CH₃CH(NH₂)CO—), Arg (arginine), Asn (asparagine), Asp (aspartic acid), Cys (cysteine), Glu (glutamic acid), H is (histidine), Ile (isoleucine), Leu (leucine; (CH₃)₂CHCH₂CH(NH₂)CO—), Lys (lysine), Met (methionine), Phe (phenylalanine), Pro (proline), Ser (serine), Thr (threonine), Trp (tryptophan), Tyr (tyrosine), Val (valine), Nva (norvaline), Hse (homoserine), 4-Hyp (4-hydroxyproline), 5-Hyl (5-hydroxylysine), Orn (ornithine) and β-Ala. Examples of suitable protecting groups include those typically employed in peptide synthesis, including acyl groups (such as formyl and acetyl), arylmethyloxycarbonyl groups (such as benzyloxycarbonyl and p-nitrobenzyloxycarbonyl), t-butoxycarbonyl groups [(CH₃)₃C—OCO—], and the like. Suitable peptide residues include petide residues comprising two to five, and optionally two to three, of the aforesaid amino acid residues. Examples of such peptide residues include, but are not limited to, residues of such peptides as Ala-Ala [CH₃CH(NH₂)CO—NHCH(CH₃)CO—], Gly-Phe, Nva-Nva, Ala-Phe, Gly-Gly, Gly-Gly-Gly, Ala-Met, Met-Met, Leu-Met and Ala-Leu. The residues of these amino acids or peptides can be present in stereochemical configurations of the D-form, the L-form or mixtures thereof. In addition, the amino acid or peptide residue may have an asymmetric carbon atom. Examples of suitable amino acid residues having an asymmetric carbon atom include residues of Ala, Leu, Phe, Trp, Nva, Val, Met, Ser, Lys, Thr and Tyr. Peptide residues having an asymmetric carbon atom include peptide residues having one or more constituent amino acid residues having an asymmetric carbon atom. Examples of suitable amino acid protecting groups include those typically employed in peptide synthesis, including acyl groups (such as formyl and acetyl), arylmethyloxycarbonyl groups (such as benzyloxycarbonyl and p-nitrobenzyloxycarbonyl), t-butoxycarbonyl groups [(CH₃)₃C—OCO—], and the like. Other examples of substituents “convertible to hydrogen in vivo” include reductively eliminable hydrogenolyzable groups. Examples of suitable reductively eliminable hydrogenolyzable groups include, but are not limited to, arylsulfonyl groups (such as o-toluenesulfonyl); methyl groups substituted with phenyl or benzyloxy (such as benzyl, trityl and benzyloxymethyl); arylmethoxycarbonyl groups (such as benzyloxycarbonyl and o-methoxy-benzyloxycarbonyl); and halogenoethoxycarbonyl groups (such as β,β,β-trichloroethoxycarbonyl and β-iodoethoxycarbonyl).

“Substituted or unsubstituted” means that a given moiety may consist of only hydrogen substituents through available valencies (unsubstituted) or may further comprise one or more non-hydrogen substituents through available valencies (substituted) that are not otherwise specified by the name of the given moiety. For example, isopropyl is an example of an ethylene moiety that is substituted by —CH₃. In general, a non-hydrogen substituent may be any substituent that may be bound to an atom of the given moiety that is specified to be substituted. Examples of substituents include, but are not limited to, aldehyde, alicyclic, aliphatic, (C₁₋₁₀)alkyl, alkylene, alkylidene, amide, amino, aminoalkyl, aromatic, aryl, bicycloalkyl, bicycloaryl, carbamoyl, carbocyclyl, carboxyl, carbonyl group, cycloalkyl, cycloalkylene, ester, halo, heterobicycloalkyl, heterocycloalkylene, heteroaryl, heterobicycloaryl, heterocycloalkyl, oxo, hydroxy, iminoketone, ketone, nitro, oxaalkyl, and oxoalkyl moieties, each of which may optionally also be substituted or unsubstituted. In one particular embodiment, examples of substituents include, but are not limited to, hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl. In addition, the substituent is itself optionally substituted by a further substituent. In one particular embodiment, examples of the further substituent include, but are not limited to, hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl.

“Sulfinyl” means the radical —SO— and/or —SO—R, wherein R is hydrogen or a further substituent. It is noted that the sulfinyl radical may be further substituted with a variety of substituents to form different sulfinyl groups including sulfinic acids, sulfinamides, sulfinyl esters, and sulfoxides.

“Sulfonyl” means the radical —SO₂— and/or —SO₂—R, wherein R is hydrogen or a further substituent. It is noted that the sulfonyl radical may be further substituted with a variety of substituents to form different sulfonyl groups including sulfonic acids, sulfonamides, sulfonate esters, and sulfones.

“Therapeutically effective amount” means that amount which, when administered to an animal for treating a disease, is sufficient to effect such treatment for the disease.

“Thioalkyl” means an alkyl, as defined above, except where one or more of the carbon atoms forming the alkyl chain are replaced with sulfur atoms (—S— or —S—R, wherein R is hydrogen or a further substituent). For example, a thio(C₁₋₁₀)alkyl refers to a chain comprising between 1 and 10 carbons and one or more sulfur atoms.

“Thiocarbonyl” means the radical —C(═S)— and/or —C(═S)—R, wherein R is hydrogen or a further substituent. It is noted that the thiocarbonyl radical may be further substituted with a variety of substituents to form different thiocarbonyl groups including thioacids, thioamides, thioesters, and thioketones.

“Treatment” or “treating” means any administration of a compound of the present invention and includes:

(1) preventing the disease from occurring in an animal which may be predisposed to the disease but does not yet experience or display the pathology or symptomatology of the disease,

(2) inhibiting the disease in an animal that is experiencing or displaying the pathology or symptomatology of the diseased (i.e., arresting further development of the pathology and/or symptomatology), or

(3) ameliorating the disease in an animal that is experiencing or displaying the pathology or symptomatology of the diseased (i.e., reversing the pathology and/or symptomatology).

It is noted in regard to all of the definitions provided herein that the definitions should be interpreted as being open ended in the sense that further substituents beyond those specified may be included. Hence, a C₁ alkyl indicates that there is one carbon atom but does not indicate what are the substituents on the carbon atom. Hence, a (C₁)alkyl comprises methyl (i.e., —CH₃) as well as —CRR′R″ where R, R′, and R″ may each independently be hydrogen or a further substituent where the atom attached to the carbon is a heteroatom or cyano. Hence, CF₃, CH₂OH and CH₂CN, for example, are all (C₁)alkyls. Similarly, terms such as alkylamino and the like comprise dialkylamino and the like.

### DETAILED DESCRIPTION OF THE INVENTION

The present invention relates to compounds that may be used to activate glucokinase. The present invention also relates to pharmaceutical compositions, kits and articles of manufacture comprising such compounds. In addition, the present invention relates to methods and intermediates useful for making the compounds. Further, the present invention relates to methods of using said compounds. It is noted that the compounds of the present invention may also possess activity for other hexokinases family members and thus may be used to address disease states associated with these other family members.

Glucokinase Activators

In one of its apsects, the present invention relates to compounds that are useful as glucokinase activators. In one embodiment, glucokinase activators of the present invention comprise:

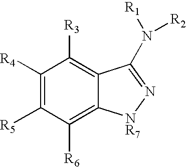

or a polymorph, solvate, ester, tautomer, enantiomer, pharmaceutically acceptable salt or prodrug thereof, wherein
R₁ is hydrogen or a substituent convertible in vivo to hydrogen;
R₂ is a substituted or unsubstituted hetero(C₂₋₁₀)aryl;
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₃)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring; and
R₇ is selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or R₇ and R₆ are taken together to form a substituted or unsubstituted ring.

In another embodiment, glucokinase activators of the present invention comprise:

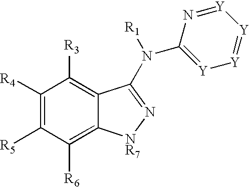

wherein
each Y is independently selected from the group consisting of CR₈ and N; and
R₈ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or two R₈ are taken together to form a substituted or unsubstituted ring.

In still another embodiment, glucokinase activators of the present invention comprise:

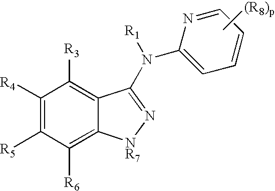

wherein
p is selected from the group consisting of 0, 1, 2, 3 and 4; and
R₈ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or two R₈ are taken together to form a substituted or unsubstituted ring.

In yet another embodiment, glucokinase activators of the present invention comprise:

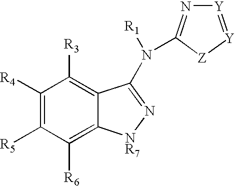

wherein
each Y is independently selected from the group consisting of CR₉ and N;
Z is selected from the group consisting of CR₁₀R₁₁, NR₁₂, S and O;
each R₉ is independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted;
R₁₀ and R₁₁ are each independently selected from the group consisting of halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted; and
R₁₂ is selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted,
or any two of R₉, R₁₀, R₁₁ and R₁₂ are taken together to form a substituted or unsubstituted ring.

In a further embodiment, glucokinase activators of the present invention comprise:

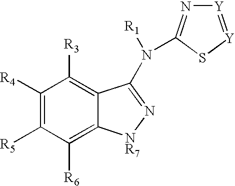

wherein
each Y is independently selected from the group consisting of CR₉ and N; and
each R₉ is independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or two R₉ are taken together to form a substituted or unsubstituted ring.

In still a further embodiment, glucokinase activators of the present invention comprise:

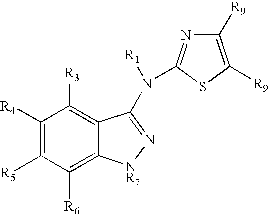

wherein
each R₉ is independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or two R₉ are taken together to form a substituted or unsubstituted ring.

In another of its apsects, the present invention relates to methods of making compounds that are useful as glucokinase activators. In one embodiment, the methods comprise the steps of:
reacting a compound comprising the formula

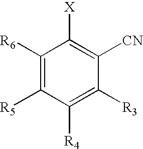

with a compound comprising the formula
H₂NNHR₇

under conditions that form a first reaction product comprising the formula 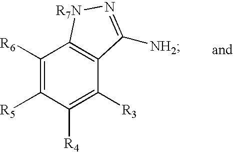
reacting the first reaction product with a compound comprising the formula
   X—R₂

under conditions that form a product comprising the formula 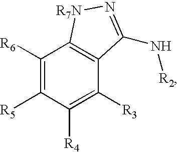
wherein
X is selected from the group consisting of F, Br, Cl and I;
R₂ is a substituted or unsubstituted hetero(C₂₋₁₀)aryl;
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring; and
R₇ is selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or R₇ and R₆ are taken together to form a substituted or unsubstituted ring.

In another embodiment, the methods comprise the steps of:
reacting a compound comprising the formula

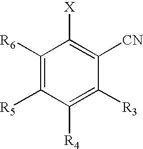

with an alcohol under conditions that form a first reaction product comprising the formula 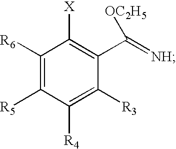
reacting the first reaction product with a compound comprising the formula
   NH₂R₁

under conditions that form a second reaction product comprising the formula 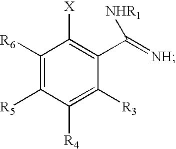
reacting the second reaction product with a compound comprising the formula
   H₂NNHR₇

under conditions that form a third reaction product comprising the formula 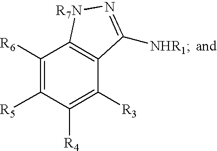
reacting the third reaction product with a compound comprising the formula
   X—R₂

under conditions that form a product comprising the formula 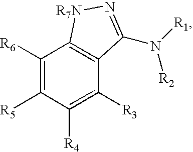
wherein
each X is independently selected from the group consisting of F, Br, Cl and I;
R₁ is hydrogen or a substituent convertible in vivo to hydrogen;
R₂ is a substituted or unsubstituted hetero(C₂₋₁₀)aryl;
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring; and
R₇ is selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or R₇ and R₆ are taken together to form a substituted or unsubstituted ring.

In still another embodiment, the methods comprise the steps of:
reacting a compound comprising the formula

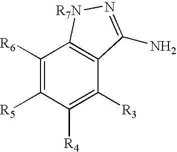

with a compound comprising the formula

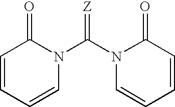

under conditions that form a first reaction product comprising the formula 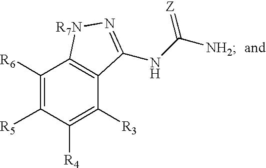
reacting the first reaction product with a compound comprising the formula

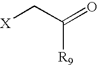

under conditions that form a product comprising the formula 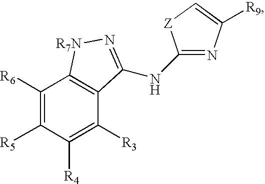
wherein
X is selected from the group consisting of F, Br, Cl and I;
Z is selected from the group consisting of CR₁₀R₁₁, NR₁₂, S and O;
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring;
R₇ is selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or R₇ and R₆ are taken together to form a substituted or unsubstituted ring;
R₉ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted;
R₁₀ and R₁₁ are each independently selected from the group consisting of halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted; and
R₁₂ is selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted.

In yet another embodiment, the methods comprise the steps of:
treating a compound comprising the formula

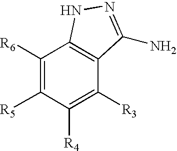

under conditions that form a first reaction product comprising the formula 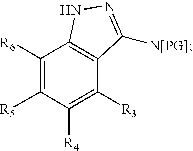
reacting the first reaction product with a compound comprising the formula
   X—R₇

under conditions that form a second reaction product comprising the formula 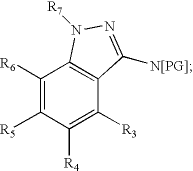
treating the second reaction product under conditions that form a third reaction product comprising the formula 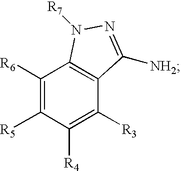
reacting the third reaction product with a compound comprising the formula

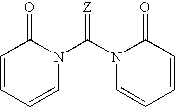

under conditions that form a fourth reaction product comprising the formula 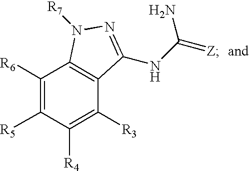
reacting the fourth reaction product with a compound comprising the formula

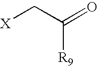

under conditions that form a product comprising the formula 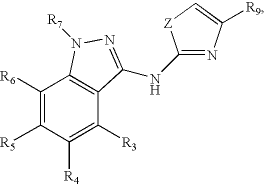
wherein
each X is independently selected from the group consisting of F, Br, Cl and I;
Z is selected from the group consisting of CR₁₀R₁₁, NR₁₂, S and O;
PG is a protecting group;
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring;
R₇ is selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or R₇ and R₆ are taken together to form a substituted or unsubstituted ring;
each R₉ is independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted;
R₁₀ and R₁₁ are each independently selected from the group consisting of halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted; and
R₁₂ is selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted.

In a further embodiment, the methods comprise the steps of:
treating a compound comprising the formula

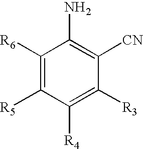

under conditions that form a first reaction product comprising the formula 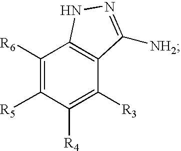
treating the first reaction product under conditions that form a second reaction product comprising the formula 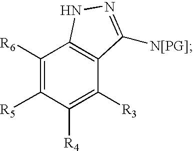
reacting the second reaction product with a compound comprising the formula
   X—R₇

under conditions that form a third reaction product comprising the formula 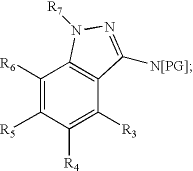
treating the third reaction product under conditions that form a fourth reaction product comprising the formula 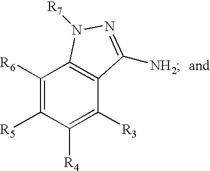
reacting the fourth reaction product with a compound comprising the formula
   X—R₂

under conditions that form a product comprising the formula 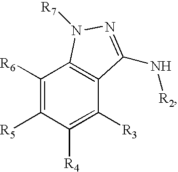
wherein
each X is independently selected from the group consisting of F, Br, Cl and I;
PG is a protecting group;
R₂ is a substituted or unsubstituted hetero(C₂₋₁₀)aryl;
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring; and
R₇ is selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or R₇ and R₆ are taken together to form a substituted or unsubstituted ring.

In still a further embodiment, the methods comprise the steps of:
reacting a compound comprising the formula

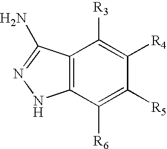

with 2,5-hexandione under conditions that form a first reaction product comprising the formula 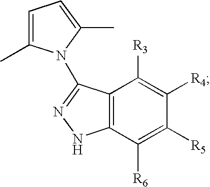
reacting the first reaction product with a compound comprising the formula
   X—R₇

under conditions that form a second reaction product comprising the formula 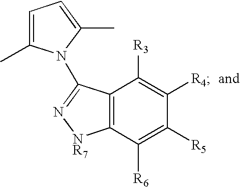
treating the second reaction product under conditions that form a product comprising the formula 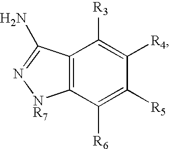
wherein
X is selected from the group consisting of F, Br, Cl and I;
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring; and
R₇ is selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or R₇ and R₆ are taken together to form a substituted or unsubstituted ring.

In yet a further embodiment, the methods comprise the steps of:
reacting a compound comprising the formula

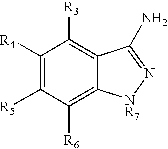

with NH₄SCN under conditions that form a first reaction product comprising the formula 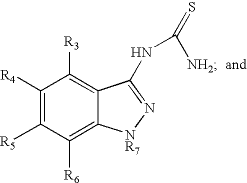
treating the first reaction product under conditions that form a product comprising the formula 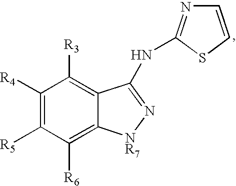
wherein
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring; and
R₇ is selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or R₇ and R₆ are taken together to form a substituted or unsubstituted ring.

In another embodiment, the methods comprise the steps of:
treating a compound comprising the formula

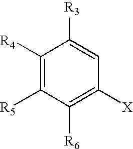

under conditions that form a first reaction product comprising the formula 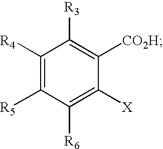
treating the first reaction product under conditions that form a second reaction product comprising the formula 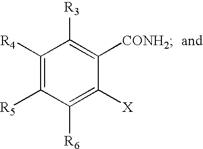
reacting the second reaction product with 2,4,6-trichloro-1,3,5-triazine under conditions that form a product comprising the formula 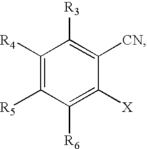
wherein
X is selected from the group consisting of F, Br, Cl and I; and
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring.

In still another embodiment, the methods comprise the steps of:
treating a compound comprising the formula

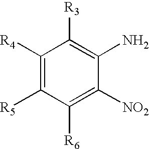

under conditions that form a first reaction product comprising the formula 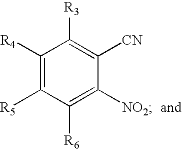
treating the first reaction product under conditions that form a product comprising the formula 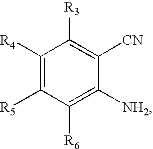
wherein
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring.

In still another of its apsects, the present invention relates to intermediates that are useful in making glucokinase activators. In one embodiment, the intermediates comprise 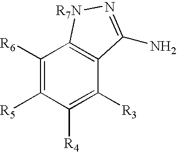
wherein
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring; and
R₇ is selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or R₇ and R₆ are taken together to form a substituted or unsubstituted ring.

In another embodiment, the intermediates comprise 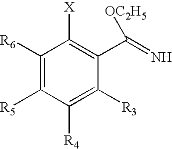
wherein
X is selected from the group consisting of F, Br, Cl and I; and
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring.

In still another embodiment, the intermediates comprise 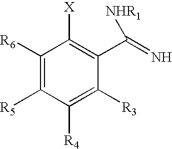
wherein
X is selected from the group consisting of F, Br, Cl and I;
R₁ is hydrogen or a substituent convertible in vivo to hydrogen; and
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring.

In yet another embodiment, the intermediates comprise 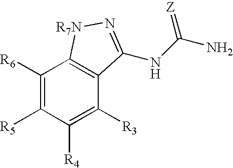
wherein
Z is selected from the group consisting of CR₁₀R₁₁, NR₁₂, S and O;
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring;
R₇ is selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or R₇ and R₆ are taken together to form a substituted or unsubstituted ring;
R₁₀ and R₁₁ are each independently selected from the group consisting of halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted; and
R₁₂ is selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted.

In a further embodiment, the intermediates comprise 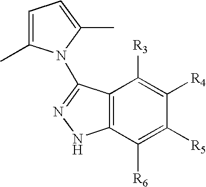
wherein
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring.

In still a further embodiment, the intermediates comprise 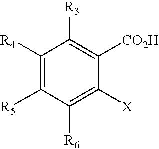
wherein
X is selected from the group consisting of F, Br, Cl and I; and
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring.

In yet a further embodiment, the intermediates comprise 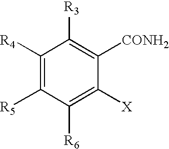
wherein
X is selected from the group consisting of F, Br, Cl and I; and
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring.

In another embodiment, the intermediates comprise 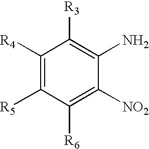
wherein
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring.

In still another embodiment, the intermediates comprise 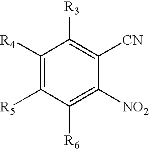
wherein
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring.

In one variation of each of the above embodiments, Z is S.

In another variation of each of the above embodiments and variations, R₁ is hydrogen.

In still another variation of each of the above embodiments and variations, R₂ is a substituted or unsubstituted hetero(C₂₋₁₀)aryl comprising a heteroatom at the 2-position. In yet another variation of each of the above embodiments and variations, R₂ is a substituted or unsubstituted hetero(C₂₋₁₀)aryl comprising at least one nitrogen. In one particular variation, the nitrogen is at the 2-position. In a further variation of each of the above embodiments and variations, R₂ is a substituted or unsubstituted hetero(C₂₋₁₀)aryl comprising an H-bond acceptor. In one particular variation, the H-bond acceptor is at the 2-position. In still a further variation of each of the above embodiments and variations, R₂ is 2-thiazolyl.

In yet a further variation of each of the above embodiments and variations, R₃ is selected from the group consisting of hydrogen, halo, nitro, oxy, hydroxy, alkoxy, amino and (C₁₋₅)alkyl, each substituted or unsubstituted. In another variation of each of the above embodiments and variations, R₃ is selected from the group consisting of hydrogen, (C₁₋₅)alkyl and halo(C₁₋₅)alkyl. In still another variation of each of the above embodiments and variations, R₃ is alkoxy. In yet another variation of each of the above embodiments and variations, R₃ is methoxy. In a further variation of each of the above embodiments and variations, R₃ is 2-methoxy.

In still a further variation of each of the above embodiments and variations, R₄ is selected from the group consisting of hydrogen, halo, nitro, oxy, hydroxy, alkoxy, amino and (C₁₋₅)alkyl, each substituted or unsubstituted. In yet a further variation of each of the above embodiments and variations, R₄ is selected from the group consisting of hydrogen, halo, nitro, hydroxy, (C₁₋₅)alkyl, halo(C₁₋₅)alkyl, hydroxy(C₁₋₅)alkyl, (C₁₋₅)alkoxy, (C₁₋₅)alkoxy-(C₁₋₅)alkoxy, (C₁₋₅)alkyl-carbonyl, amino and (C₁₋₅)alkyl-carbonylamino, each substituted or unsubstituted. In another variation of each of the above embodiments and variations, R₄ is alkoxy. In still another variation of each of the above embodiments and variations, R₄ is methoxy. In yet another variation of each of the above embodiments and variations, R₄ is 2-methoxy. In a further variation of each of the above embodiments and variations, R₄ is —CF₃.

In still a further variation of each of the above embodiments and variations, R₅ is selected from the group consisting of hydrogen, halo, nitro, oxy, hydroxy, alkoxy, amino and (C₁₋₅)alkyl, each substituted or unsubstituted. In yet a further variation of each of the above embodiments and variations, R₅ is hydrogen. In another variation of each of the above embodiments and variations, wherein R₅ is alkoxy. In still another variation of each of the above embodiments and variations, R₅ is methoxy. In a further variation of each of the above embodiments and variations, R₅ is 2-methoxy. In still a further variation of each of the above embodiments and variations, R₅ is —CF₃.

In yet a further variation of each of the above embodiments and variations, R₆ is selected from the group consisting of hydrogen, halo, nitro, oxy, hydroxy, alkoxy, aryloxy, heteroaryloxy, amino, (C₁₋₁₀)alkyl, aryl(C₁₋₁₀)alkyl, (C₄₋₁₂)aryl and hetero(C₂₋₁₀)aryl, each substituted or unsubstituted. In another variation of each of the above embodiments and variations, R₆ is selected from the group consisting of hydrogen, halo, (C₁₋₅)alkoxy, (C₁₋₅)alkyl and halo(C₁₋₅)alkyl. In still another variation of each of the above embodiments and variations, R₆ is alkoxy. In yet another variation of each of the above embodiments and variations, R₆ is methoxy. In another variation of each of the above embodiments and variations, R₆ is 2-methoxy.

In still another variation of each of the above embodiments and variations, R₇ is selected from the group consisting of hydrogen and substituted or unsubstituted (C₁₋₅)alkyl. In yet another variation of each of the above embodiments and variations, R₇ is selected from the group consisting of hydrogen, (C₁₋₅)alkyl, aza(C₁₋₅)alkyl, (mono- or di-(C₁₋₅)alkylamino)(C₁₋₅)alkyl and (C₁₋₅)alkoxy-carbonyl-amino(C₁₋₅)alkyl, each substituted or unsubstituted.

In a further variation of each of the above embodiments and variations, R₈ is selected from the group consisting of halo, oxycarbonyl, carboxy, carboxamido, acetoxy, (C₁₋₅)alkyl, hydroxy(C₁₋₅)alkyl, aza(C₁₋₅)alkyl and acetylamino(C₁₋₅)alkyl, each substituted or unsubstituted. In still a further variation of each of the above embodiments and variations, R₈ is methyl. In yet a further variation of each of the above embodiments and variations, R₈ is —C(O)—O—R₁₃, wherein R₁₃ is selected from the group consisting of a substituted or unsubstituted (C₁₋₁₀)alkyl.

In another variation of each of the above embodiments and variations, R₉ is selected from the group consisting of halo, oxycarbonyl, carboxy, carboxamido, acetoxy, (C₁₋₅)alkyl, hydroxy(C₁₋₅)alkyl, aza(C₁₋₅)alkyl and acetylamino(C₁₋₅)alkyl, each substituted or unsubstituted. In still another variation of each of the above embodiments and variations, R₉ is methyl. In yet another variation of each of the above embodiments and variations, R₉ is —C(O)—O—R₁₃, wherein R₁₃ is selected from the group consisting of a substituted or unsubstituted (C₁₋₁₀)alkyl.

In a further variation of each of the above embodiments and variations, R₁₀ is selected from the group consisting of halo, oxycarbonyl, carboxy, carboxamido, acetoxy, (C₁₋₅)alkyl, hydroxy(C₁₋₅)alkyl, aza(C₁₋₅)alkyl and acetylamino(C₁₋₅)alkyl, each substituted or unsubstituted. In still a further variation of each of the above embodiments and variations, R₁₀ is methyl. In yet a further variation of each of the above embodiments and variations, R₁₀ is —C(O)—O—R₁₃, wherein R₁₃ is selected from the group consisting of a substituted or unsubstituted (C₁₋₁₀)alkyl.

In another variation of each of the above embodiments and variations, X is Br. In still another variation of each of the above embodiments and variations, X is Cl.

In yet another variation of each of the above embodiments and variations, PG is a substituted or unsubstituted benzyl. In a further variation of each of the above embodiments and variations, PG together with the N to which it is attached forms a substituted or unsubstituted pyrrole.

In still another variation of each of the above embodiments and variations,
R₁ is hydrogen;
R₂ is 2-thiazolyl;
R₃ is hydrogen, (C₁₋₅)alkyl or halo-(C₁₋₅)alkyl;
R₄ is hydrogen, halo, nitro, hydroxy, (C₁₋₅)alkyl, halo(C₁₋₅)alkyl, hydroxy(C₁₋₅)alkyl, (C₁₋₅)alkoxy, (C₁₋₅)alkoxy-(C₁₋₅)alkoxy, (C₁₋₅)alkyl-carbonyl, amino or (C₁₋₅)alkyl-carbonylamino;
R₅ is hydrogen;
R₆ is hydrogen, halo, (C₁₋₅)alkoxy, (C₁₋₅)alkyl or halo(C₁₋₅)alkyl; and
R₇ is hydrogen, (C₁₋₅)alkyl, aza(C₁₋₅)alkyl, (mono- or di-(C₁₋₅)alkylamino)-(C₁₋₅)alkyl or (C₁₋₅)alkoxy-carbonylamino-(C₁₋₅)alkyl.

Particular examples of compounds according to the present invention include, but are not limited to:
thiazol-2-yl-(5-trifluoromethyl-1H-indazol-3-yl)-amine;
(4-phenyl-thiazol-2-yl)-(5-trifluoromethyl-1H-indazol-3-yl)-amine;
2-(5-Trifluoromethyl-1H-indazol-3-ylamino)-thiazole-4-carboxylic acid ethyl ester;
(4-phenyl-thiazol-2-yl)-(6-trifluoromethyl-1H-indazol-3-yl)-amine;
4-methyl-N-(5-(trifluoromethyl)-1H-indazol-3-yl)thiazol-2-amine;
N-(1-benzyl-5-trifluoromethyl-1H-indazol-3-yl)-thiazol-2-yl-amine;
5-Bromo-N-1,3-thiazol-2-yl-1H-indazol-3-amine;
5-Chloro-N-1,3-thiazol-2-yl-1H-indazol-3-amine;
1-Methyl-N-1,3-thiazol-2-yl-5-(trifluoromethyl)-1H-indazol-3-amine;
1-Ethyl-N-1,3-thiazol-2-yl-5-(trifluoromethyl)-1H-indazol-3-amine;
1-Isobutyl-N-1,3-thiazol-2-yl-5-(trifluoromethyl)-1H-indazol-3-amine;
tert-Butyl {3-[3-(1,3-thiazol-2-ylamino)-5-(trifluoromethyl)-1H-indazol-1-yl]propyl}carbamate;
1-(3-Aminopropyl)-N-1,3-thiazol-2-yl-5-(trifluoromethyl)-1H-indazol-3-amine;
1-[3-(Dimethylamino)propyl]-N-1,3-thiazol-2-yl-5-(trifluoromethyl)-1H-indazol-3-amine;
tert-Butyl {4-[3-(1,3-thiazol-2-ylamino)-5-(trifluoromethyl)-1H-indazol-1-yl]butyl}carbamate;
1-(4-Aminobutyl)-N-1,3-thiazol-2-yl-5-(trifluoromethyl)-1H-indazol-3-amine;
1-[4-(Dimethylamino)butyl]-N-1,3-thiazol-2-yl-5-(trifluoromethyl)-1H-indazol-3-amine;
[3-(1,3-Thiazol-2-ylamino)-1H-indazol-5-yl]methanol;
5-Ethyl-N-1,3-thiazol-2-yl-1H-indazol-3-amine;
5-Nitro-N-1,3-thiazol-2-yl-1H-indazol-3-amine;
N³-1,3-Thiazol-2-yl-1H-indazole-3,5-diamine;
N-[3-(1,3-Thiazol-2-ylamino)-1H-indazol-5-yl]acetamide;
N-1,3-Thiazol-2-yl-7-(trifluoromethyl)-1H-indazol-3-amine;
7-Fluoro-N-1,3-thiazol-2-yl-1H-indazol-3-amine;
1-[3-(1,3-Thiazol-2-ylamino)-1H-indazol-5-yl]ethanone;
5-Methoxy-N-1,3-thiazol-2-yl-1H-indazol-3-amine;
5-Propyl-N-1,3-thiazol-2-yl-1H-indazol-3-amine;
3-(1,3-Thiazol-2-ylamino)-1H-indazol-5-ol;
5-Isobutyl-N-1,3-thiazol-2-yl-1H-indazol-3-amine;
N-1,3-Thiazol-2-yl-4-(trifluoromethyl)-1H-indazol-3-amine;
7-Bromo-5-propyl-N-1,3-thiazol-2-yl-1H-indazol-3-amine;
4,5-Dimethyl-N-1,3-thiazol-2-yl-1H-indazol-3-amine;
5-Isopropyl-N-1,3-thiazol-2-yl-1H-indazol-3-amine;
5-Isopropoxy-N-1,3-thiazol-2-yl-1H-indazol-3-amine;
5-(2-Methoxy-1-methylethoxy)-N-1,3-thiazol-2-yl-1H-indazol-3-amine; and
5-Isobutyl-7-methoxy-N-1,3-thiazol-2-yl-1H-indazol-3-amine.

In one particular embodiment, examples of compounds according to the present invention include, but are not limited to:
5-bromo-N-1,3-thiazol-2-yl-1H-indazol-3-amine;
1-methyl-N-1,3-thiazol-2-yl-5-(trifluoromethyl)-1H-indazol-3-amine;
5-nitro-N-1,3-thiazol-2-yl-1H-indazol-3-amine;
N-[3-(1,3-thiazol-2-ylamino)-1H-indazol-5-yl]acetamide;
1-[3-(1,3-thiazol-2-ylamino)-1H-indazol-5-yl]ethanone;
5-isobutyl-N-1,3-thiazol-2-yl-1H-indazol-3-amine;
5-isopropyl-N-1,3-thiazol-2-yl-1H-indazol-3-amine;
5-isopropoxy-N-1,3-thiazol-2-yl-1H-indazol-3-amine; and
5-(2-methoxy-1-methylethoxy)-N-1,3-thiazol-2-yl-1H-indazol-3-amine.

It is noted that the compounds of the present invention may be in the form of a pharmaceutically acceptable salt, biohydrolyzable ester, biohydrolyzable amide, biohydrolyzable carbamate, solvate, hydrate or prodrug thereof. For example, the compound optionally comprises a substituent that is convertible in vivo to a different substituent such as hydrogen.

It is further noted that the compound may be present in a mixture of stereoisomers, or the compound may comprise a single stereoisomer.

The present invention also provides a pharmaceutical composition comprising as an active ingredient a compound according to any one of the above embodiments and variations. In one particular variation, the composition is a solid formulation adapted for oral administration. In another particular variation, the composition is a liquid formulation adapted for oral administration. In yet another particular variation, the composition is a tablet. In still another particular variation, the composition is a liquid formulation adapted for parenteral administration.

In another of its aspects, there is provided a pharmaceutical composition comprising a compound according to any one of the above embodiments and variations, wherein the composition is adapted for administration by a route selected from the group consisting of orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, and intrathecally.

In yet another of its aspects, there is provided a kit comprising a compound of any one of the above embodiments and variations; and instructions which comprise one or more forms of information selected from the group consisting of indicating a disease state for which the composition is to be administered, storage information for the composition, dosing information and instructions regarding how to administer the composition. In one particular variation, the kit comprises the compound in a multiple dose form.

In still another of its aspects, there is provided an article of manufacture comprising a compound of any one of the above embodiments and variations; and packaging materials. In one variation, the packaging material comprises a container for housing the compound. In one particular variation, the container comprises a label indicating one or more members of the group consisting of a disease state for which the compound is to be administered, storage information, dosing information and/or instructions regarding how to administer the compound. In another variation, the article of manufacture comprises the compound in a multiple dose form.

In a further of its aspects, there is provided a therapeutic method comprising administering a compound of any one of the above embodiments and variations to a subject.

In another of its aspects, there is provided a method of activating glucokinase comprising contacting glucokinase with a compound of any one of the above embodiments and variations.

In yet another of its aspects, there is provided a method of activating glucokinase comprising causing a compound of any one of the above embodiments and variations to be present in a subject in order to activate glucokinase in vivo.

In a further of its aspects, there is provided a method of activating glucokinase comprising administering a first compound to a subject that is converted in vivo to a second compound wherein the second compound activates glucokinase in vivo, the second compound being a compound according to any one of the above embodiments and variations.

In another of its aspects, there is provided a method of treating a disease state for which increasing glucokinase activity ameliorates the pathology and/or symptomology of the disease state, the method comprising causing a compound of any one of the above embodiments and variations to be present in a subject in a therapeutically effective amount for the disease state.

In yet another of its aspects, there is provided a method of treating a disease state for which increasing glucokinase activity ameliorates the pathology and/or symptomology of the disease state, the method comprising administering a compound of any one of the above embodiments and variations to a subject, wherein the compound is present in the subject in a therapeutically effective amount for the disease state.

In a further of its aspects, there is provided a method of treating a disease state for which increasing glucokinase activity ameliorates the pathology and/or symptomology of the disease state, the method comprising administering a first compound to a subject that is converted in vivo to a second compound wherein the second compound activates glucokinase in vivo, the second compound being a compound according to any one of the above embodiments and variations.

In one variation of each of the above methods the disease state is selected from the group consisting of hyperglycemia, diabetes (e.g., type-1 diabetes, type-2 diabetes, gestational diabetes, obesity diabetes), dyslipidaemia (e.g., hypertriglyceridemia, hypercholesterolemia, hypo-HDL-emia, postprandial hyperlipidemia), obesity, insulin resistance, metabolic syndrome, syndrome X, impaired glucose tolerance, polycystic ovary syndrome and cardiovascular disease (including arteriosclerosis).

Salts, Hydrates, and Prodrugs of Glucokinase Activators

It should be recognized that the compounds of the present invention may be present and optionally administered in the form of salts, hydrates and prodrugs that are converted in vivo into the compounds of the present invention. For example, it is within the scope of the present invention to convert the compounds of the present invention into and use them in the form of their pharmaceutically acceptable salts derived from various organic and inorganic acids and bases in accordance with procedures well known in the art.

When the compounds of the present invention possess a free base form, the compounds can be prepared as a pharmaceutically acceptable acid addition salt by reacting the free base form of the compound with a pharmaceutically acceptable inorganic or organic acid, e.g., hydrohalides such as hydrochloride, hydrobromide, hydroiodide; other mineral acids and their corresponding salts such as sulfate, nitrate, phosphate, etc.; and alkyl and monoarylsulfonates such as ethanesulfonate, toluenesulfonate and benzenesulfonate; and other organic acids and their corresponding salts such as acetate, tartrate, maleate, succinate, citrate, benzoate, salicylate and ascorbate. Further acid addition salts of the present invention include, but are not limited to: adipate, alginate, arginate, aspartate, bisulfate, bisulfite, bromide, butyrate, camphorate, camphorsulfonate, caprylate, chloride, chlorobenzoate, cyclopentanepropionate, digluconate, dihydrogenphosphate, dinitrobenzoate, dodecylsulfate, fumarate, galacterate (from mucic acid), galacturonate, glucoheptaoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, iso-butyrate, lactate, lactobionate, malate, malonate, mandelate, metaphosphate, methanesulfonate, methylbenzoate, monohydrogenphosphate, 2-naphthalenesulfonate, nicotinate, nitrate, oxalate, oleate, pamoate, pectinate, persulfate, phenylacetate, 3-phenylpropionate, phosphate, phosphonate and phthalate. It should be recognized that the free base forms will typically differ from their respective salt forms somewhat in physical properties such as solubility in polar solvents, but otherwise the salts are equivalent to their respective free base forms for the purposes of the present invention.

When the compounds of the present invention possess a free acid form, a pharmaceutically acceptable base addition salt can be prepared by reacting the free acid form of the compound with a pharmaceutically acceptable inorganic or organic base. Examples of such bases are alkali metal hydroxides including potassium, sodium and lithium hydroxides; alkaline earth metal hydroxides such as barium and calcium hydroxides; alkali metal alkoxides, e.g., potassium ethanolate and sodium propanolate; and various organic bases such as ammonium hydroxide, piperidine, diethanolamine and N-methylglutamine. Also included are the aluminum salts of the compounds of the present invention. Further base salts of the present invention include, but are not limited to: copper, ferric, ferrous, lithium, magnesium, manganic, manganous, potassium, sodium and zinc salts. Organic base salts include, but are not limited to, salts of primary, secondary and tertiary amines, substituted amines including naturally occurring substituted amines, cyclic amines and basic ion exchange resins, e.g., arginine, betaine, caffeine, chloroprocaine, choline, N,N′-dibenzylethylenediamine (benzathine), dicyclohexylamine, diethanolamine, 2-diethylaminoethanol, 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris-(hydroxymethyl)-methylamine (tromethamine). It should be recognized that the free acid forms will typically differ from their respective salt forms somewhat in physical properties such as solubility in polar solvents, but otherwise the salts are equivalent to their respective free acid forms for the purposes of the present invention.

Compounds of the present invention that comprise basic nitrogen-containing groups may be quaternized with such agents as (C₁₋₄) alkyl halides, e.g., methyl, ethyl, iso-propyl and tert-butyl chlorides, bromides and iodides; di (C₁₋₄) alkyl sulfates, e.g., dimethyl, diethyl and diamyl sulfates; (C₁₀₋₁₈) alkyl halides, e.g., decyl, dodecyl, lauryl, myristyl and stearyl chlorides, bromides and iodides; and aryl (C₁₋₄) alkyl halides, e.g., benzyl chloride and phenethyl bromide. Such salts permit the preparation of both water-soluble and oil-soluble compounds of the present invention.

N-oxides of compounds according to the present invention can be prepared by methods known to those of ordinary skill in the art. For example, N-oxides can be prepared by treating an unoxidized form of the compound with an oxidizing agent (e.g., trifluoroperacetic acid, permaleic acid, perbenzoic acid, peracetic acid, meta-chloroperoxybenzoic acid, or the like) in a suitable inert organic solvent (e.g., a halogenated hydrocarbon such as dichloromethane) at approximately 0° C. Alternatively, the N-oxides of the compounds can be prepared from the N-oxide of an appropriate starting material.

Prodrug derivatives of compounds according to the present invention can be prepared by modifying substituents of compounds of the present invention that are then converted in vivo to a different substituent. It is noted that in many instances, the prodrugs themselves also fall within the scope of the range of compounds according to the present invention. For example, prodrugs can be prepared by reacting a compound with a carbamylating agent (e.g., 1,1-acyloxyalkylcarbonochloridate, para-nitrophenyl carbonate, or the like) or an acylating agent. Further examples of methods of making prodrugs are described in Saulnier et al. (1994), Bioorganic and Medicinal Chemistry Letters, Vol. 4, p. 1985.

Protected derivatives of compounds of the present invention can also be made. Examples of techniques applicable to the creation of protecting groups and their removal can be found in T. W. Greene, Protecting Groups in Organic Synthesis, 3^{rd} edition, John Wiley & Sons, Inc. 1999.

Compounds of the present invention may also be conveniently prepared, or formed during the process of the invention, as solvates (e.g., hydrates). Hydrates of compounds of the present invention may be conveniently prepared by recrystallization from an aqueous/organic solvent mixture, using organic solvents such as dioxin, tetrahydrofuran or methanol.

A “pharmaceutically acceptable salt”, as used herein, is intended to encompass any compound according to the present invention that is utilized in the form of a salt thereof, especially where the salt confers on the compound improved pharmacokinetic properties as compared to the free form of compound or a different salt form of the compound. The pharmaceutically acceptable salt form may also initially confer desirable pharmacokinetic properties on the compound that it did not previously possess, and may even positively affect the pharmacodynamics of the compound with respect to its therapeutic activity in the body. An example of a pharmacokinetic property that may be favorably affected is the manner in which the compound is transported across cell membranes, which in turn may directly and positively affect the absorption, distribution, biotransformation and excretion of the compound. While the route of administration of the pharmaceutical composition is important, and various anatomical, physiological and pathological factors can critically affect bioavailability, the solubility of the compound is usually dependent upon the character of the particular salt form thereof, which it utilized. One of skill in the art will appreciate that an aqueous solution of the compound will provide the most rapid absorption of the compound into the body of a subject being treated, while lipid solutions and suspensions, as well as solid dosage forms, will result in less rapid absorption of the compound.

Compositions Comprising Glucokinase Activators

A wide variety of compositions and administration methods may be used in conjunction with the compounds of the present invention. Such compositions may include, in addition to the compounds of the present invention, conventional pharmaceutical excipients, and other conventional, pharmaceutically inactive agents. Additionally, the compositions may include active agents in addition to the compounds of the present invention. These additional active agents may include additional compounds according to the invention, and/or one or more other pharmaceutically active agents.

The compositions may be in gaseous, liquid, semi-liquid or solid form, formulated in a manner suitable for the route of administration to be used. For oral administration, capsules and tablets are typically used. For parenteral administration, reconstitution of a lyophilized powder, prepared as described herein, is typically used.

Compositions comprising compounds of the present invention may be administered or coadministered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compounds and/or compositions according to the invention may also be administered or coadministered in slow release dosage forms.

The glucokinase activators and compositions comprising them may be administered or coadministered in any conventional dosage form. Co-administration in the context of this invention is intended to mean the administration of more than one therapeutic agent, one of which includes a glucokinase activator, in the course of a coordinated treatment to achieve an improved clinical outcome. Such co-administration may also be coextensive, that is, occurring during overlapping periods of time.

Solutions or suspensions used for parenteral, intradermal, subcutaneous, or topical application may optionally include one or more of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; agents for the adjustment of tonicity such as sodium chloride or dextrose, and agents for adjusting the acidity or alkalinity of the composition, such as alkaline or acidifying agents or buffers like carbonates, bicarbonates, phosphates, hydrochloric acid, and organic acids like acetic and citric acid. Parenteral preparations may optionally be enclosed in ampules, disposable syringes or single or multiple dose vials made of glass, plastic or other suitable material.

When compounds according to the present invention exhibit insufficient solubility, methods for solubilizing the compounds may be used. Such methods are known to those of skill in this art, and include, but are not limited to, using cosolvents, such as dimethylsulfoxide (DMSO), using surfactants, such as TWEEN, or dissolution in aqueous sodium bicarbonate. Derivatives of the compounds, such as prodrugs of the compounds may also be used in formulating effective pharmaceutical compositions.

Upon mixing or adding compounds according to the present invention to a composition, a solution, suspension, emulsion or the like may be formed. The form of the resulting composition will depend upon a number of factors, including the intended mode of administration, and the solubility of the compound in the selected carrier or vehicle. The effective concentration needed to ameliorate the disease being treated may be empirically determined.

Compositions according to the present invention are optionally provided for administration to humans and animals in unit dosage forms, such as tablets, capsules, pills, powders, dry powders for inhalers, granules, sterile parenteral solutions or suspensions, and oral solutions or suspensions, and oil-water emulsions containing suitable quantities of the compounds, particularly the pharmaceutically acceptable salts, preferably the sodium salts, thereof. The pharmaceutically therapeutically active compounds and derivatives thereof are typically formulated and administered in unit-dosage forms or multiple-dosage forms. Unit-dose forms, as used herein, refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes individually packaged tablet or capsule. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pint or gallons. Hence, multiple dose form is a multiple of unit-doses that are not segregated in packaging.

In addition to one or more compounds according to the present invention, the composition may comprise: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polyinylpyrrolidine, celluloses and derivatives thereof, povidone, crospovidones and other such binders known to those of skill in the art. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to form a solution or suspension. If desired, the pharmaceutical composition to be administered may also contain minor amounts of auxiliary substances such as wetting agents, emulsifying agents, or solubilizing agents, pH buffering agents and the like, for example, acetate, sodium citrate, cyclodextrine derivatives, sorbitan monolaurate, triethanolamine sodium acetate, triethanolamine oleate, and other such agents. Actual methods of preparing such dosage forms are known in the art, or will be apparent, to those skilled in this art; for example, see Remington's Pharmaceutical Sciences, Mack Publishing Company, Easton, Pa., 15th Edition, 1975. The composition or formulation to be administered will, in any event, contain a sufficient quantity of an activator of the present invention to increase glucokinase activity in vivo, thereby treating the disease state of the subject.

Dosage forms or compositions may optionally comprise one or more compounds according to the present invention in the range of 0.005% to 100% (weight/weight) with the balance comprising additional substances such as those described herein. For oral administration, a pharmaceutically acceptable composition may optionally comprise any one or more commonly employed excipients, such as, for example pharmaceutical grades of mannitol, lactose, starch, magnesium stearate, talcum, cellulose derivatives, sodium crosscarmellose, glucose, sucrose, magnesium carbonate, sodium saccharin, talcum. Such compositions include solutions, suspensions, tablets, capsules, powders, dry powders for inhalers and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparing these formulations are known to those skilled in the art. The compositions may optionally contain 0.01%-100% (weight/weight) of one or more glucokinase activators, optionally 0.1-95%, and optionally 1-95%.

Salts, preferably sodium salts, of the activators may be prepared with carriers that protect the compound against rapid elimination from the body, such as time release formulations or coatings. The formulations may further include other active compounds to obtain desired combinations of properties.

Formulations for Oral Administration

Oral pharmaceutical dosage forms may be as a solid, gel or liquid. Examples of solid dosage forms include, but are not limited to tablets, capsules, granules, and bulk powders. More specific examples of oral tablets include compressed, chewable lozenges and tablets that may be enteric-coated, sugar-coated or film-coated. Examples of capsules include hard or soft gelatin capsules. Granules and powders may be provided in non-effervescent or effervescent forms. Each may be combined with other ingredients known to those skilled in the art.

In certain embodiments, compounds according to the present invention are provided as solid dosage forms, preferably capsules or tablets. The tablets, pills, capsules, troches and the like may optionally contain one or more of the following ingredients, or compounds of a similar nature: a binder; a diluent; a disintegrating agent; a lubricant; a glidant; a sweetening agent; and a flavoring agent.

Examples of binders that may be used include, but are not limited to, microcrystalline cellulose, gum tragacanth, glucose solution, acacia mucilage, gelatin solution, sucrose and starch paste.

Examples of lubricants that may be used include, but are not limited to, talc, starch, magnesium or calcium stearate, lycopodium and stearic acid.

Examples of diluents that may be used include, but are not limited to, lactose, sucrose, starch, kaolin, salt, mannitol and dicalcium phosphate.

Examples of glidants that may be used include, but are not limited to, colloidal silicon dioxide.

Examples of disintegrating agents that may be used include, but are not limited to, crosscarmellose sodium, sodium starch glycolate, alginic acid, corn starch, potato starch, bentonite, methylcellulose, agar and carboxymethylcellulose.

Examples of coloring agents that may be used include, but are not limited to, any of the approved certified water-soluble FD and C dyes, mixtures thereof, and water insoluble FD and C dyes suspended on alumina hydrate.

Examples of sweetening agents that may be used include, but are not limited to, sucrose, lactose, mannitol and artificial sweetening agents such as sodium cyclamate and saccharin, and any number of spray-dried flavors.

Examples of flavoring agents that may be used include, but are not limited to, natural flavors extracted from plants such as fruits and synthetic blends of compounds that produce a pleasant sensation, such as, but not limited to peppermint and methyl salicylate.

Examples of wetting agents that may be used include, but are not limited to, propylene glycol monostearate, sorbitan monooleate, diethylene glycol monolaurate and polyoxyethylene lauryl ether.

Examples of anti-emetic coatings that may be used include, but are not limited to, fatty acids, fats, waxes, shellac, ammoniated shellac and cellulose acetate phthalates.

Examples of film coatings that may be used include, but are not limited to, hydroxyethylcellulose, sodium carboxymethylcellulose, polyethylene glycol 4000 and cellulose acetate phthalate.

If oral administration is desired, the salt of the compound may optionally be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it may optionally additionally comprise a liquid carrier such as a fatty oil. In addition, dosage unit forms may optionally additionally comprise various other materials that modify the physical form of the dosage unit, for example, coatings of sugar and other enteric agents.

Compounds according to the present invention may also be administered as a component of an elixir, suspension, syrup, wafer, sprinkle, chewing gum or the like. A syrup may optionally comprise, in addition to the active compounds, sucrose as a sweetening agent and certain preservatives, dyes and colorings and flavors.

The compounds of the present invention may also be mixed with other active materials that do not impair the desired action, or with materials that supplement the desired action, such as antacids, H2 blockers, and diuretics. For example, if a compound is used for treating asthma or hypertension, it may be used with other bronchodilators and antihypertensive agents, respectively.

Examples of pharmaceutically acceptable carriers that may be included in tablets comprising compounds of the present invention include, but are not limited to binders, lubricants, diluents, disintegrating agents, coloring agents, flavoring agents, and wetting agents. Enteric-coated tablets, because of the enteric-coating, resist the action of stomach acid and dissolve or disintegrate in the neutral or alkaline intestines. Sugar-coated tablets may be compressed tablets to which different layers of pharmaceutically acceptable substances are applied. Film-coated tablets may be compressed tablets that have been coated with polymers or other suitable coating. Multiple compressed tablets may be compressed tablets made by more than one compression cycle utilizing the pharmaceutically acceptable substances previously mentioned. Coloring agents may also be used in tablets. Flavoring and sweetening agents may be used in tablets, and are especially useful in the formation of chewable tablets and lozenges.

Examples of liquid oral dosage forms that may be used include, but are not limited to, aqueous solutions, emulsions, suspensions, solutions and/or suspensions reconstituted from non-effervescent granules and effervescent preparations reconstituted from effervescent granules.

Examples of aqueous solutions that may be used include, but are not limited to, elixirs and syrups. As used herein, elixirs refer to clear, sweetened, hydroalcoholic preparations. Examples of pharmaceutically acceptable carriers that may be used in elixirs include, but are not limited to solvents. Particular examples of solvents that may be used include glycerin, sorbitol, ethyl alcohol and syrup. As used herein, syrups refer to concentrated aqueous solutions of a sugar, for example, sucrose. Syrups may optionally further comprise a preservative.

Emulsions refer to two-phase systems in which one liquid is dispersed in the form of small globules throughout another liquid. Emulsions may optionally be oil-in-water or water-in-oil emulsions. Examples of pharmaceutically acceptable carriers that may be used in emulsions include, but are not limited to non-aqueous liquids, emulsifying agents and preservatives.

Examples of pharmaceutically acceptable substances that may be used in non-effervescent granules, to be reconstituted into a liquid oral dosage form, include diluents, sweeteners and wetting agents.

Examples of pharmaceutically acceptable substances that may be used in effervescent granules, to be reconstituted into a liquid oral dosage form, include organic acids and a source of carbon dioxide.

Coloring and flavoring agents may optionally be used in all of the above dosage forms.

Particular examples of preservatives that may be used include glycerin, methyl and propylparaben, benzoic add, sodium benzoate and alcohol.

Particular examples of non-aqueous liquids that may be used in emulsions include mineral oil and cottonseed oil.

Particular examples of emulsifying agents that may be used include gelatin, acacia, tragacanth, bentonite, and surfactants such as polyoxyethylene sorbitan monooleate.

Particular examples of suspending agents that may be used include sodium carboxymethylcellulose, pectin, tragacanth, Veegum and acacia. Diluents include lactose and sucrose. Sweetening agents include sucrose, syrups, glycerin and artificial sweetening agents such as sodium cyclamate and saccharin.

Particular examples of wetting agents that may be used include propylene glycol monostearate, sorbitan monooleate, diethylene glycol monolaurate and polyoxyethylene lauryl ether.

Particular examples of organic acids that may be used include citric and tartaric acid.

Sources of carbon dioxide that may be used in effervescent compositions include sodium bicarbonate and sodium carbonate. Coloring agents include any of the approved certified water soluble FD and C dyes, and mixtures thereof.

Particular examples of flavoring agents that may be used include natural flavors extracted from plants such fruits, and synthetic blends of compounds that produce a pleasant taste sensation.

For a solid dosage form, the solution or suspension, in for example propylene carbonate, vegetable oils or triglycerides, is preferably encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. Re 28,819 and 4,358,603.

Injectables, Solutions, and Emulsions

The present invention is also directed to compositions designed to administer the compounds of the present invention by parenteral administration, generally characterized by subcutaneous, intramuscular or intravenous injection. Injectables may be prepared in any conventional form, for example as liquid solutions or suspensions, solid forms suitable for solution or suspension in liquid prior to injection, or as emulsions.

Examples of excipients that may be used in conjunction with injectables according to the present invention include, but are not limited to water, saline, dextrose, glycerol or ethanol. The injectable compositions may also optionally comprise minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) is also contemplated herein. The percentage of active compound contained in such parenteral compositions is highly dependent on the specific nature thereof, as well as the activity of the compound and the needs of the subject.

Parenteral administration of the formulations includes intravenous, subcutaneous and intramuscular administrations. Preparations for parenteral administration include sterile solutions ready for injection, sterile dry soluble products, such as the lyophilized powders described herein, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

When administered intravenously, examples of suitable carriers include, but are not limited to physiological saline or phosphate buffered saline (PBS), and solutions containing thickening and solubilizing agents, such as glucose, polyethylene glycol, and polypropylene glycol and mixtures thereof.

Examples of pharmaceutically acceptable carriers that may optionally be used in parenteral preparations include, but are not limited to aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles that may optionally be used include Sodium Chloride Injection, Ringers Injection, Isotonic Dextrose Injection, Sterile Water Injection, Dextrose and Lactated Ringers Injection.

Examples of nonaqueous parenteral vehicles that may optionally be used include fixed oils of vegetable origin, cottonseed oil, corn oil, sesame oil and peanut oil.

Antimicrobial agents in bacteriostatic or fungistatic concentrations may be added to parenteral preparations, particularly when the preparations are packaged in multiple-dose containers and thus designed to be stored and multiple aliquots to be removed. Examples of antimicrobial agents that may be used include phenols or cresols, mercurials, benzyl alcohol, chlorobutanol, methyl and propyl p-hydroxybenzoic acid esters, thimerosal, benzalkonium chloride and benzethonium chloride.

Examples of isotonic agents that may be used include sodium chloride and dextrose. Examples of buffers that may be used include phosphate and citrate. Examples of antioxidants that may be used include sodium bisulfate. Examples of local anesthetics that may be used include procaine hydrochloride. Examples of suspending and dispersing agents that may be used include sodium carboxymethylcellulose, hydroxypropyl methylcellulose and polyvinylpyrrolidone. Examples of emulsifying agents that may be used include Polysorbate 80 (TWEEN 80). A sequestering or chelating agent of metal ions includes EDTA.

Pharmaceutical carriers may also optionally include ethyl alcohol, polyethylene glycol and propylene glycol for water miscible vehicles and sodium hydroxide, hydrochloric acid, citric acid or lactic acid for pH adjustment.

The concentration of an activator in the parenteral formulation may be adjusted so that an injection administers a pharmaceutically effective amount sufficient to produce the desired pharmacological effect. The exact concentration of an activator and/or dosage to be used will ultimately depend on the age, weight and condition of the patient or animal as is known in the art.

Unit-dose parenteral preparations may be packaged in an ampoule, a vial or a syringe with a needle. All preparations for parenteral administration should be sterile, as is know and practiced in the art.

Injectables may be designed for local and systemic administration. Typically a therapeutically effective dosage is formulated to contain a concentration of at least about 0.1% w/w up to about 90% w/w or more, preferably more than 1% w/w of the glucokinase activator to the treated tissue(s). The activator may be administered at once, or may be divided into a number of smaller doses to be administered at intervals of time. It is understood that the precise dosage and duration of treatment will be a function of the location of where the composition is parenterally administered, the carrier and other variables that may be determined empirically using known testing protocols or by extrapolation from in vivo or in vitro test data. It is to be noted that concentrations and dosage values may also vary with the age of the individual treated. It is to be further understood that for any particular subject, specific dosage regimens may need to be adjusted over time according to the individual need and the professional judgment of the person administering or supervising the administration of the formulations. Hence, the concentration ranges set forth herein are intended to be exemplary and are not intended to limit the scope or practice of the claimed formulations.

The glucokinase activator may optionally be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the disease state and may be empirically determined.

Lyophilized Powders

The compounds of the present invention may also be prepared as lyophilized powders, which can be reconstituted for administration as solutions, emulsions and other mixtures. The lyophilized powders may also be formulated as solids or gels.

Sterile, lyophilized powder may be prepared by dissolving the compound in a sodium phosphate buffer solution containing dextrose or other suitable excipient. Subsequent sterile filtration of the solution followed by lyophilization under standard conditions known to those of skill in the art provides the desired formulation. Briefly, the lyophilized powder may optionally be prepared by dissolving dextrose, sorbitol, fructose, corn syrup, xylitol, glycerin, glucose, sucrose or other suitable agent, about 1-20%, preferably about 5 to 15%, in a suitable buffer, such as citrate, sodium or potassium phosphate or other such buffer known to those of skill in the art at, typically, about neutral pH. Then, a glucokinase activator is added to the resulting mixture, preferably above room temperature, more preferably at about 30-35° C., and stirred until it dissolves. The resulting mixture is diluted by adding more buffer to a desired concentration. The resulting mixture is sterile filtered or treated to remove particulates and to insure sterility, and apportioned into vials for lyophilization. Each vial may contain a single dosage or multiple dosages of the activator.

Topical Administration

The compounds of the present invention may also be administered as topical mixtures. Topical mixtures may be used for local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The glucokinase activators may be formulated as aerosols for topical application, such as by inhalation (see, U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will typically have diameters of less than 50 microns, preferably less than 10 microns.

The activators may also be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the glucokinase activator alone or in combination with other pharmaceutically acceptable excipients can also be administered.

Formulations for Other Routes of Administration

Depending upon the disease state being treated, other routes of administration, such as topical application, transdermal patches, and rectal administration, may also be used. For example, pharmaceutical dosage forms for rectal administration are rectal suppositories, capsules and tablets for systemic effect. Rectal suppositories are used herein mean solid bodies for insertion into the rectum that melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax, (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is about 2 to 3 gm. Tablets and capsules for rectal administration may be manufactured using the same pharmaceutically acceptable substance and by the same methods as for formulations for oral administration.

Examples of Formulations

The following are particular examples of oral, intravenous and tablet formulations that may optionally be used with compounds of the present invention. It is noted that these formulations may be varied depending on the particular compound being used and the indication for which the formulation is going to be used.

ORAL FORMULATION

| | | |
|---|---|---|
| Compound of the Present Invention | 10-100 | mg |
| Citric Acid Monohydrate | 105 | mg |
| Sodium Hydroxide | 18 | mg |
| Flavoring | | |
| Water | q.s. to 100 | mL |

INTRAVENOUS FORMULATION

| Compound of the Present Invention | 0.1-10 | mg |
|---|---|---|
| Dextrose Monohydrate | q.s. to make isotonic | |
| Citric Acid Monohydrate | 1.05 | mg |
| Sodium Hydroxide | 0.18 | mg |
| Water for Injection | q.s. to 1.0 | mL |

TABLET FORMULATION

| | |
|---|---|
| Compound of the Present Invention | 1% |
| Microcrystalline Cellulose | 73% |
| Stearic Acid | 25% |
| Colloidal Silica | 1%. |

Kits Comprising Glucokinase Activators

The invention is also directed to kits and other articles of manufacture for treating diseases associated with glucokinase. It is noted that diseases are intended to cover all conditions for which increasing glucokinase activity (e.g., upregulation of glucokinase) ameliorates the pathology and/or symptomology of the condition.

In one embodiment, a kit is provided that comprises a composition comprising at least one activator of the present invention in combination with instructions. The instructions may indicate the disease state for which the composition is to be administered, storage information, dosing information and/or instructions regarding how to administer the composition. The kit may also comprise packaging materials. The packaging material may comprise a container for housing the composition. The kit may also optionally comprise additional components, such as syringes for administration of the composition. The kit may comprise the composition in single or multiple dose forms.

In another embodiment, an article of manufacture is provided that comprises a composition comprising at least one activator of the present invention in combination with packaging materials. The packaging material may comprise a container for housing the composition. The container may optionally comprise a label indicating the disease state for which the composition is to be administered, storage information, dosing information and/or instructions regarding how to administer the composition. The kit may also optionally comprise additional components, such as syringes for administration of the composition. The kit may comprise the composition in single or multiple dose forms.

It is noted that the packaging material used in kits and articles of manufacture according to the present invention may form a plurality of divided containers such as a divided bottle or a divided foil packet. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container that is employed will depend on the exact dosage form involved, for example a conventional cardboard box would not generally be used to hold a liquid suspension. It is feasible that more than one container can be used together in a single package to market a single dosage form. For example, tablets may be contained in a bottle that is in turn contained within a box. Typically the kit includes directions for the administration of the separate components. The kit form is particularly advantageous when the separate components are preferably administered in different dosage forms (e.g., oral, topical, transdermal and parenteral), are administered at different dosage intervals, or when titration of the individual components of the combination is desired by the prescribing physician.

One particular example of a kit according to the present invention is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

Another specific embodiment of a kit is a dispenser designed to dispense the daily doses one at a time in the order of their intended use. Preferably, the dispenser is equipped with a memory-aid, so as to further facilitate compliance with the regimen. An example of such a memory-aid is a mechanical counter that indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

Dosage, Host and Safety

The compounds of the present invention are stable and can be used safely. In particlar, the compounds of the present invention are useful as glucokinase activators for a variety of subjects (e.g., humans, non-human mammals and non-mammals). The optimal dose may vary depending upon such conditions as, for example, the type of subject, the body weight of the subject, the route of administration, and specific properties of the particular compound being used. In general, the daily dose for oral administration to an adult (body weight of about 60 kg) is about 0.01 to 100 mg/kg body weight, about 0.05 to 30 mg/kg body weight, or about 0.1 to 10 mg/kg body weight, for oral administration to adult diabetic patients, which is desirably administered in one to three portions a day.

Combination Therapies

A wide variety of therapeutic agents may have a therapeutic additive or synergistic effect with GK activators according to the present invention. In particular, the present invention also relates to the use of the GK activators of the present invention in combination with one or more other antidiabetic compounds. Examples of such other antidiabetic compounds include, but are not limited to S9 proteases, like dipeptidyl peptidase IV (DPP-IV) inhibitors; insulin signaling pathway modulators, like protein tyrosine phosphatase (PTPase) inhibitors, and glutamine-fructose-6-phosphate amidotransferase (GFAT) inhibitors; compounds influencing a dysregulated hepatic glucose production, like glucose-6-phosphatase (G6Pase) inhibitors, fructose-1,6-bisphosphatase (F-1,6-BPase) inhibitors, glycogen phosphorylase (GP) inhibitors, glucagon receptor antagonists and phosphoenolpyruvate carboxykinase (PEPCK) inhibitors; pyruvate dehydrogenase kinase (PDHK) inhibitors; insulin sensitivity enhancers (insulin sensitizers); insulin secretion enhancers (insulin secretagogues); alpha-glucosidase inhibitors; inhibitors of gastric emptying; other glucokinase (GK) activators; GLP-1 receptor agonists; UCP modulators; RXR modulators; GSK-3 inhibitors; PPAR modulators; biguanides; insulin; and α₂-adrenergic antagonists. The compound of the present invention may be administered with such at least one other antidiabetic compound either simultaneously as a single dose, at the same time as separate doses, or sequentially (i.e., where one is administered before or after the other is administered). Examples of DPP-IV inhibitors include vildagliptin, sitagliptin phosphate and saxagliptin. Examples of insulin sensitivity enhancers (insulin sensitizers) include pioglitazone or a salt thereof (preferably hydrochloride), rosiglitazone or a salt thereof (preferably maleate), netoglitazone, edaglitazone, rivoglitazone, tesaglitazar, ragaglitazar, muraglitazar, metaglidasen, naveglitazar and balaglitazone. Examples of insulin secretion enhancers (insulin secretagogues) include sulfonylureas (e.g., tolbutamide, glibenclamide, gliclazide, chlorpropamide, tolazamide, acetohexamide, glyclopyramide, glimepiride, glipizide, glybuzole), repaglinide, senaglinide, nateglinide and mitiglinide or calcium salt hydrate thereof. Examples of alpha-glucosidase inhibitors include voglibose, acarbose and miglitol. Examples of GLP-1 receptor agonists include GLP-1, GLP-1MR agent and exendin-4. Examples of biguanides include metformin, buformin and their salts (e.g., hydrochloride).

In the case of combination therapy with compounds of the present invention, the other antidiabetic compound may be administered (e.g., route and dosage form) in a manner known per se for such compound. Compounds of the present invention and the other antidiabetic compound may be administered sequentially (i.e., at separate times) or at the same time, either one after the other separately in two separate dose forms or in one combined, single dose form. In one particular embodiment, the other antidiabetic compound is administered with compounds of the present invention as a single, combined dosage form. The dose of the antidiabetic compound may be selected from the range known to be clinically employed for such compound. Any of the therapeutic compounds of diabetic complications, antihyperlipemic compounds or antiobestic compounds can be used in combination with compounds of the present invention in the same manner as the above antidiabetic compounds.

### EXAMPLES

Preparation of Glucokinase Activators

Various methods may be developed for synthesizing compounds according to the present invention. Representative methods for synthesizing these compounds are provided in the Examples. It is noted, however, that the compounds of the present invention may also be synthesized by other synthetic routes that others may devise.

It will be readily recognized that certain compounds according to the present invention have atoms with linkages to other atoms that confer a particular stereochemistry to the compound (e.g., chiral centers). It is recognized that synthesis of compounds according to the present invention may result in the creation of mixtures of different stereoisomers (i.e., enantiomers and diastereomers). Unless a particular stereochemistry is specified, recitation of a compound is intended to encompass all of the different possible stereoisomers.

Various methods for separating mixtures of different stereoisomers are known in the art. For example, a racemic mixture of a compound may be reacted with an optically active resolving agent to form a pair of diastereoisomeric compounds. The diastereomers may then be separated in order to recover the optically pure enantiomers. Dissociable complexes may also be used to resolve enantiomers (e.g., crystalline diastereoisomeric salts). Diastereomers typically have sufficiently distinct physical properties (e.g., melting points, boiling points, solubilities, reactivity, etc.) and can be readily separated by taking advantage of these dissimilarities. For example, diastereomers can typically be separated by chromatography or by separation/resolution techniques based upon differences in solubility. A more detailed description of techniques that can be used to resolve stereoisomers of compounds from their racemic mixture can be found in Jean Jacques Andre Collet, Samuel H. Wilen, Enantiomers, Racemates and Resolutions, John Wiley & Sons, Inc. (1981).

Compounds according to the present invention can also be prepared as a pharmaceutically acceptable acid addition salt by reacting the free base form of the compound with a pharmaceutically acceptable inorganic or organic acid. Alternatively, a pharmaceutically acceptable base addition salt of a compound can be prepared by reacting the free acid form of the compound with a pharmaceutically acceptable inorganic or organic base. Inorganic and organic acids and bases suitable for the preparation of the pharmaceutically acceptable salts of compounds are set forth in the definitions section of this application. Alternatively, the salt forms of the compounds can be prepared using salts of the starting materials or intermediates.

The free acid or free base forms of the compounds can be prepared from the corresponding base addition salt or acid addition salt form. For example, a compound in an acid addition salt form can be converted to the corresponding free base by treating with a suitable base (e.g., ammonium hydroxide solution, sodium hydroxide, and the like). A compound in a base addition salt form can be converted to the corresponding free acid by treating with a suitable acid (e.g., hydrochloric acid, etc).

The N-oxides of compounds according to the present invention can be prepared by methods known to those of ordinary skill in the art. For example, N-oxides can be prepared by treating an unoxidized form of the compound with an oxidizing agent (e.g., trifluoroperacetic acid, permaleic acid, perbenzoic acid, peracetic acid, meta-chloroperoxybenzoic acid, or the like) in a suitable inert organic solvent (e.g., a halogenated hydrocarbon such as dichloromethane) at approximately 0° C. Alternatively, the N-oxides of the compounds can be prepared from the N-oxide of an appropriate starting material.

Compounds in an unoxidized form can be prepared from N-oxides of compounds by treating with a reducing agent (e.g., sulfur, sulfur dioxide, triphenyl phosphine, lithium borohydride, sodium borohydride, phosphorus trichloride, tribromide, or the like) in an suitable inert organic solvent (e.g., acetonitrile, ethanol, aqueous dioxane, or the like) at 0 to 80° C.

Prodrug derivatives of the compounds can be prepared by methods known to those of ordinary skill in the art (e.g., for further details see Saulnier et al. (1994), Bioorganic and Medicinal Chemistry Letters, Vol. 4, p. 1985). For example, appropriate prodrugs can be prepared by reacting a non-derivatized compound with a suitable carbamylating agent (e.g., 1,1-acyloxyalkylcarbonochloridate, para-nitrophenyl carbonate, or the like).

Protected derivatives of the compounds can be made by methods known to those of ordinary skill in the art. A detailed description of the techniques applicable to the creation of protecting groups and their removal can be found in T. W. Greene, Protecting Groups in Organic Synthesis, 3^{rd} edition, John Wiley & Sons, Inc. 1999.

Compounds according to the present invention may be conveniently prepared, or formed during the process of the invention, as solvates (e.g., hydrates). Hydrates of compounds of the present invention may be conveniently prepared by recrystallization from an aqueous/organic solvent mixture, using organic solvents such as dioxin, tetrahydrofuran or methanol.

Compounds according to the present invention can also be prepared as their individual stereoisomers by reacting a racemic mixture of the compound with an optically active resolving agent to form a pair of diastereoisomeric compounds, separating the diastereomers and recovering the optically pure enantiomer. While resolution of enantiomers can be carried out using covalent diastereomeric derivatives of compounds, dissociable complexes are preferred (e.g., crystalline diastereoisomeric salts). Diastereomers have distinct physical properties (e.g., melting points, boiling points, solubilities, reactivity, etc.) and can be readily separated by taking advantage of these dissimilarities. The diastereomers can be separated by chromatography or, preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques Andre Collet, Samuel H. Wilen, Enantiomers, Racemates and Resolutions, John Wiley & Sons, Inc. (1981).

As used herein the symbols and conventions used in these processes, schemes and examples are consistent with those used in the contemporary scientific literature, for example, the Journal of the American Chemical Society or the Journal of Biological Chemistry. Standard single-letter or thee-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

| | |
|---|---|
| μL (microliters) | Ac (acetyl) |
| atm (atmosphere) | ATP (Adenosine Triphophatase) |
| BOC (tert-butyloxycarbonyl) | BOP (bis(2-oxo-3-oxazolidinyl)phosphinic |
| | chloride) |
| BSA (Bovine Serum Albumin) | CBZ (benzyloxycarbonyl) |
| CDI (1,1-carbonyldiimidazole) | DCC (dicyclohexylcarbodiimide) |
| DCE (dichloroethane) | DCM (dichloromethane) |
| DMAP (4-dimethylaminopyridine) | DME (1,2-dimethoxyethane) |
| DMF (N,N-dimethylformamide) | DMPU (N,N′-dimethylpropyleneurea) |
| DMSO (dimethylsulfoxide) | EDCI (ethylcarbodiimide hydrochloride) |
| EDTA (Ethylenediaminetetraacetic acid) | Et (ethyl) |
| Et₂O (diethyl ether) | EtOAc (ethyl acetate) |
| FMOC (9-fluorenylmethoxycarbonyl) | g (grams) |
| h (hours) | HOAc or AcOH (acetic acid) |
| HOBT (1-hydroxybenzotriazole) | HOSu (N-hydroxysuccinimide) |
| HPLC (high pressure liquid chromatography) | Hz (Hertz) |
| i.v. (intravenous) | IBCF (isobutyl chloroformate) |
| i-PrOH (isopropanol) | L (liters) |
| M (molar) | mCPBA (meta-chloroperbenzoic acid) |
| Me (methyl) | MeOH (methanol) |
| mg (milligrams) | MHz (megahertz) |
| min (minutes) | mL (milliliters) |
| mM (millimolar) | mmol (millimoles) |
| mol (moles) | MOPS (Morpholinepropanesulfonic acid) |
| mp (melting point) | NaOAc (sodium acetate) |
| OMe (methoxy) | psi (pounds per square inch) |
| RP (reverse phase) | RT (ambient temperature) |
| SPA (Scintillation Proximity Assay) | TBAF (tetra-n-butylammonium fluoride) |
| TBS (t-butyldimethylsilyl) | tBu (tert-butyl) |
| TEA (triethylamine) | TFA (trifluoroacetic acid) |
| TFAA (trifluoroacetic anhydride) | THF (tetrahydrofuran) |
| TIPS (triisopropylsilyl) | TLC (thin layer chromatography) |
| TMS (trimethylsilyl) | TMSE (2-(trimethylsilyl)ethyl) |
| Tr (retention time) | |

All references to ether or Et₂O are to diethyl ether; and brine refers to a saturated aqueous solution of NaCl. Unless otherwise indicated, all temperatures are expressed in ° C. (degrees Centigrade). All reactions are conducted under an inert atmosphere at RT unless otherwise noted.

¹H NMR spectra were recorded on a Bruker Avance 400. Chemical shifts are expressed in parts per million (ppm). Coupling constants are in units of Hertz (Hz). Splitting patterns describe apparent multiplicities and are designated as s (singlet), d (doublet), t (triplet), q (quartet), m (multiplet), br (broad).

Low-resolution mass spectra (MS) and compound purity data were acquired on a Waters ZQ LC/MS single quadrupole system equipped with electrospray ionization (ESI) source, UV detector (220 and 254 nm), and evaporative light scattering detector (ELSD). Thin-layer chromatography was performed on 0.25 mm E. Merck silica gel plates (60F-254), visualized with UV light, 5% ethanolic phosphomolybdic acid, Ninhydrin or p-anisaldehyde solution. Flash column chromatography was performed on silica gel (230-400 mesh, Merck).

The starting materials and reagents used in preparing these compounds are either available from commercial suppliers such as the Aldrich Chemical Company (Milwaukee, Wis.), Bachem (Torrance, Calif.), Sigma (St. Louis, Mo.), or may be prepared by methods well known to a person of ordinary skill in the art, following procedures described in such standard references as Fieser and Fieser's Reagents for Organic Synthesis, vols. 1-17, John Wiley and Sons, New York, N.Y., 1991; Rodd's Chemistry of Carbon Compounds, vols. 1-5 and supps., Elsevier Science Publishers, 1989; Organic Reactions, vols. 1-40, John Wiley and Sons, New York, N.Y., 1991; March J.: Advanced Organic Chemistry, 4th ed., John Wiley and Sons, New York, N.Y.; and Larock: Comprehensive Organic Transformations, VCH Publishers, New York, 1989.

The entire disclosures of all documents cited throughout this application are incorporated herein by reference.

Synthetic Schemes for Compounds of the Present Invention

Compounds according to the present invention may be synthesized according to the reaction schemes shown below. Other reaction schemes could be readily devised by those skilled in the art. It should also be appreciated that a variety of different solvents, temperatures and other reaction conditions can be varied to optimize the yields of the reactions.

In the reactions described hereinafter it may be necessary to protect reactive functional groups, for example hydroxy, amino, imino, thio or carboxy groups, where these are desired in the final product, to avoid their unwanted participation in the reactions. Conventional protecting groups may be used in accordance with standard practice, for examples see T. W. Greene and P. G. M. Wuts in “Protective Groups in Organic Chemistry” John Wiley and Sons, 1991.

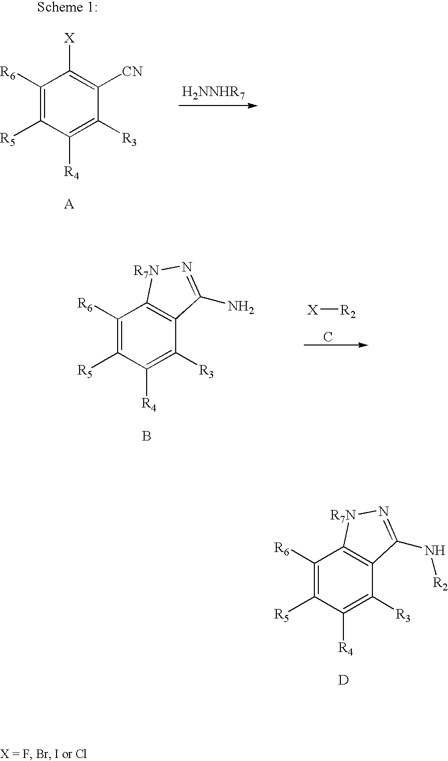

Referring to Scheme 1, the 1H-indazol-3-ylamine B is prepared from compound A using the procedure described in U.S. Pat. No. 3,133,081 (Lafferty et al.), which is hereby incorporated by reference in its entirety. Compound B and compound C are heated in, for example, n-butanol. The product is optionally concentrated in vacuo and purified by, for example, silica gel chromatography (e.g., 1:1:1 EtOAc/hexanes/CH₂Cl₂) to give compound D.

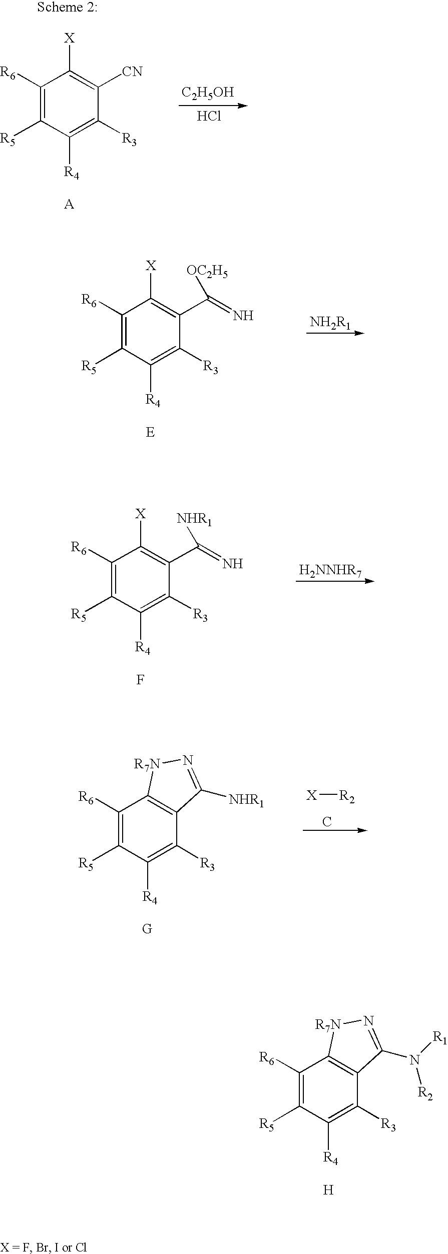

Referring to Scheme 2, the 1H-indazol-3-ylamine G is prepared from compound A using the procedure described in U.S. Pat. No. 3,133,081 (Lafferty et al.), which is hereby incorporated by reference in its entirety. Compound G and compound C are heated in, for example, n-butanol. The product is optionally concentrated in vacuo and purified by, for example, silica gel chromatography (e.g., 1:1:1 EtOAc/hexanes/CH₂Cl₂) to give compound H.

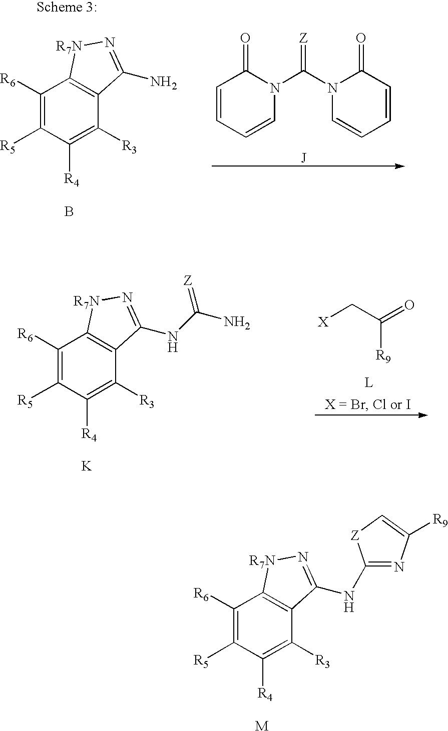

Referring to Scheme 3, compound J is added to a solution of compound B in CH₂Cl₂ and the reaction stirred. The solution is then added to a mixture of aq. NH₄OH in MeOH and the reaction stirred. Organics can be extracted with CHCl₃, dried (e.g., MgSO₄), and concentrated in vacuo to give compound K. Compound L is added to a solution of compound K in EtOH and the reaction stirred at reflux. The solution is then cooled, washed with brine, dried (e.g., MgSO₄), and concentrated in vacuo. The product is optionally purified by, for example, silica gel chromatography (e.g., 1:1:1 EtOAc/hexanes/CH₂Cl₂) to yield compound M.

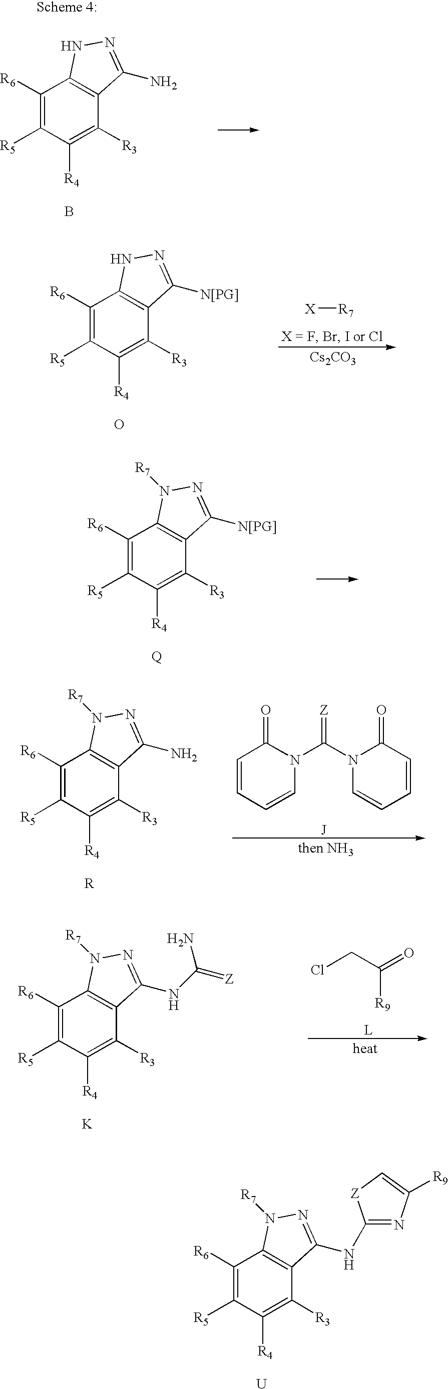

Referring to Scheme 4, compound O is prepared by protecting the amine of compound B using any of a variety of suitable protecting groups (PG), such as, for example, 2,5-hexandione. A mixture of compound O, compound P and Cs₂CO₃ in NMP is stirred and the product worked-up to yield compound Q. The amine of compound Q is then deprotected to provide compound R. Progress of the reaction can be monitored using, for example, LC/MS. Compound K is prepared from compound R according to a procedure analogous to that described in connection with Scheme 3. Compound U is prepared from compound S according to a procedure analogous to that described in connection with Scheme 3.

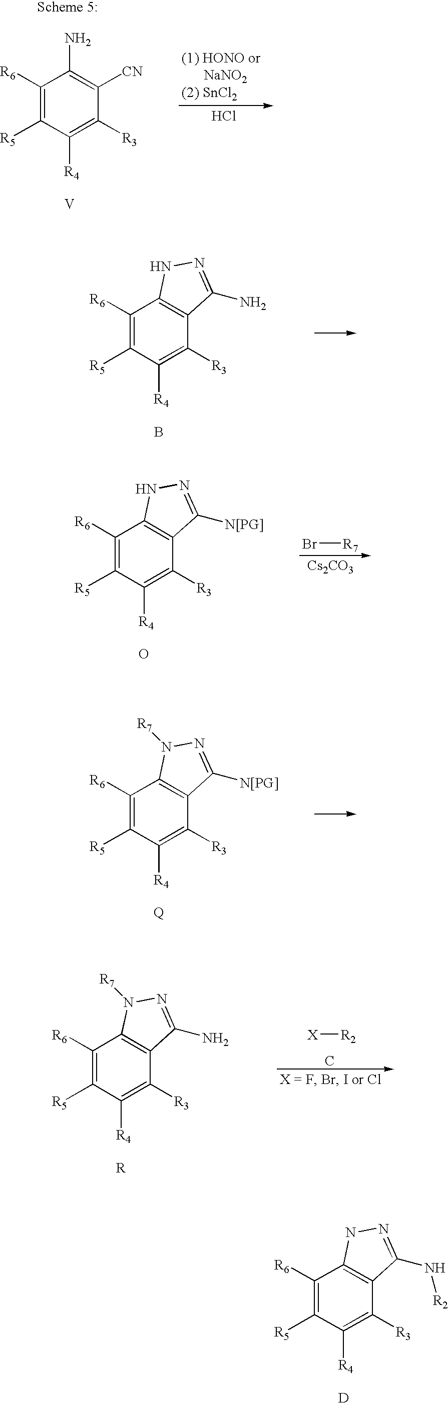

Referring to Scheme 5, the 1H-indazol-3-ylamine B is prepared from compound V using the procedure described in U.S. Pat. No. 3,133,081 (Lafferty et al.), which is hereby incorporated by reference in its entirety. Compound R is prepared from compound B according to the procedure described in connection with Scheme 4. Compound R is reacted with compound C as described in connection with Scheme 2. The product is optionally concentrated in vacuo and purified by, for example, silica gel chromatography (e.g., 1:1:1 EtOAc/hexanes/CH₂Cl₂) to give compound D.

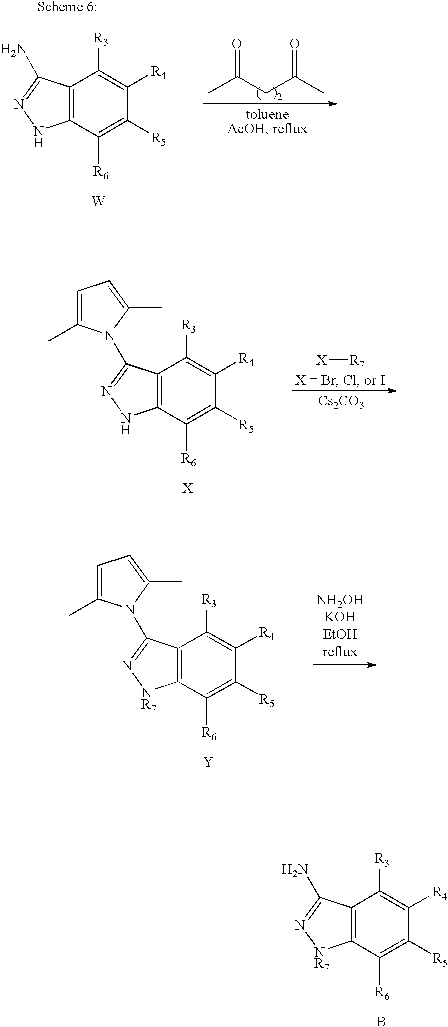

Referring to Scheme 6, compound W is reacted with 2,5-hexandione to provide compound X. The reaction product is optionally washed (e.g., with water) and dried (e.g., MgSO₄). Compound X is then reacted with X—R₇ to provide compound Y. The mixture can be diluted (e.g., with water), extracted (e.g., with EtOAc) and dried (e.g., MgSO₄). Compound Y is treated with NH₂OH.HCl and KOH to provide compound B. The mixture is optionally extracted (e.g., with DCM) and dried (e.g., MgSO₄).

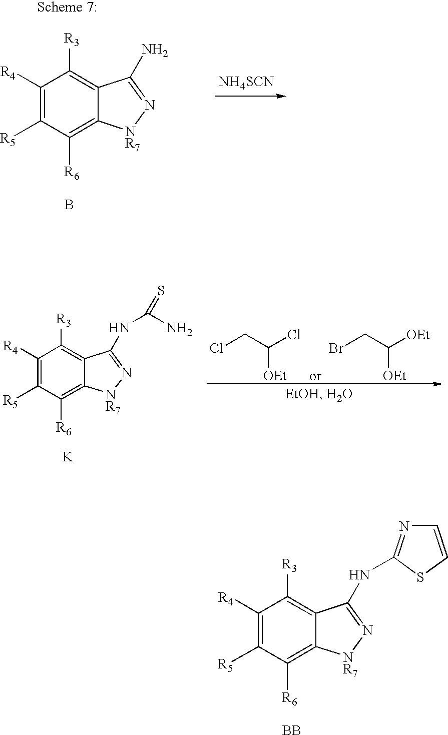

Referring to Scheme 7, compound B is treated with ammonium thiocyanate to provide compound K. Compound K is then reacted with 1,2-dichloro-1-ethoxyethane or 2-bromo-1,1-diethoxyethane to obtain compound BB. The mixture is optionally washed (e.g., with water and brine), dried (e.g., MgSO₄), filtered and/or concentrated in vacuo.

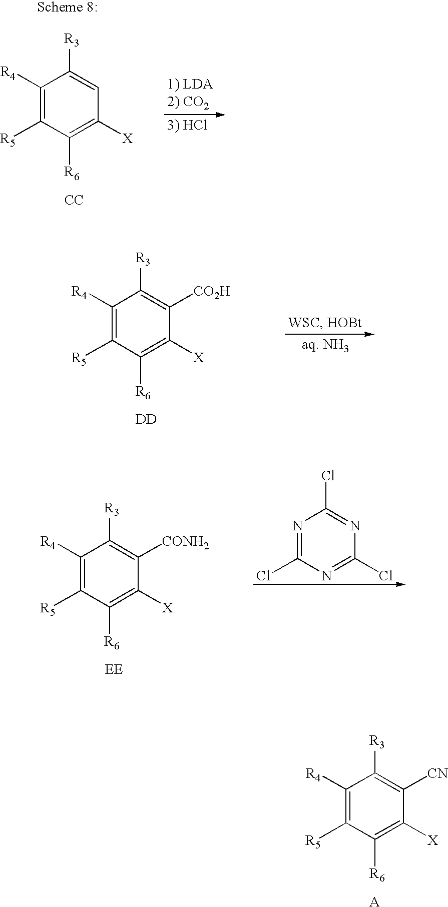

Referring to Scheme 8, compound CC is mixed with lithium diispropylamide. Carbon dioxide is then bubbled through the mixture and the mixture is then treated with an acid (e.g., HCl). The organic layer is extracted (e.g., with ethyl acetate) and optionally washed (e.g., with brine), dried (e.g., MgSO₄), filtered and/or concentyrated in vacuo to provide compound DD. Compound DD is treated with water-soluble carbodiimide and 1-hydroxybenzotriazole in ammonia to provide compound EE. Compound EE is reacted with cyanuric chloride to obtain compound A.

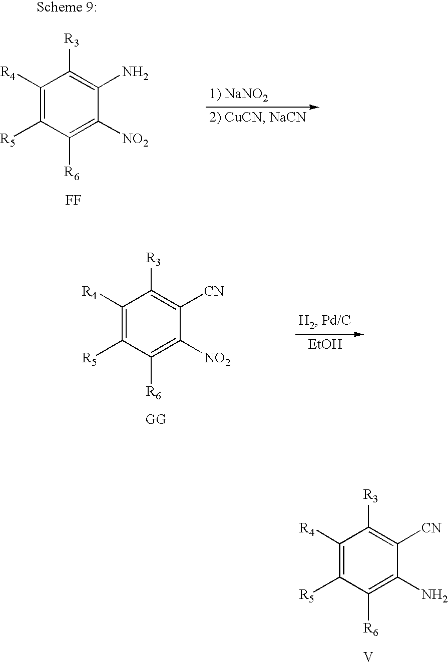

Referring to Scheme 9, compound FF is treated with sodium nitrite and reacted with copper cyamide and sodium cyamide to provide compound GG. Compound GG is treated with palladium charcoal under hydrogen atmosphere to obtain compound V.

Chiral components can be separated and purified using any of a variety of techniques known to those skilled in the art. For example, chiral components can be purified using supercritical fluid chromatography (SFC). In one particular variation, chiral analytical SFC/MS analyses are conducted using a Berger analytical SFC system (AutoChem, Newark, Del.) which consists of a Berger SFC dual pump fluid control module with a Berger FCM 1100/1200 supercritical fluid pump and FCM 1200 modifier fluid pump, a Berger TCM 2000 oven, and an Alcott 718 autosampler. The integrated system can be controlled by BI-SFC Chemstation software version 3.4. Detection can be accomplished with a Watrers ZQ 2000 detector operated in positive mode with an ESI interface and a scan range from 200-800 Da with 0.5 second per scan. Chromatographic separations can be performed on a ChiralPak AD-H, ChiralPak AS-H, ChiralCel OD-H, or ChiralCel OJ-H column (5μ, 4.6×250 mm; Chiral Technologies, Inc. West Chester, Pa.) with 10 to 40% methanol as the modifier and with or without ammonium acetate (10 mM). Any of a variety of flow rates can be utilized including, for example, 1.5 or 3.5 mL/min with an inlet pressure set at 100 bar. Additionally, a variety of sample injection conditions can be used including, for example, sample injections of either 5 or 10 μL in methanol at 0.1 mg/mL in concentration.

In another variation, preparative chiral separations are performed using a Berger MultiGram II SFC purification system. For example, samples can be loaded onto a ChiralPak AD column (21×250 mm, 10μ). In particular variations, the flow rate for separation can be 70 mL/min, the injection volume up to 2 mL, and the inlet pressure set at 130 bar. Stacked injections can be applied to increase the efficiency.

In each of the above reaction procedures or schemes, the various substituents may be selected from among the various substituents otherwise taught herein.

Descriptions of the syntheses of particular compounds according to the present invention based on the above reaction scheme are set forth herein.

Examples of Glucokinase Activators

The present invention is further exemplified, but not limited by, the following examples that describe the synthesis of particular compounds according to the invention.

### Example 1

### Thiazol-2-yl-(5-trifluoromethyl-1H-indazol-3-yl)-amine

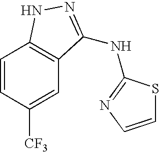

5-Trifluoromethyl-1H-indazol-3-ylamine (100 mg, 0.5 mmol) and 2-bromothiazole (90 mg, 0.55 mmol) were heated in n-butanol (5 mL) at 100° C. for 3 days. The reaction was concentrated in vacuo and purified by silica gel chromatography (1:1:1 EtOAc/hexanes/CH₂Cl₂) to give 38 mg (27%) of the title compound as a white solid. ¹H NMR (400 MHz, DMSO-d6): δ 12.78 (s, 1H), 11.50 (s, 1H), 8.64 (s, 1H), 7.57-7.64 (m, 2H), 7.37 (d, 1H, J=3.6 Hz), 7.03 (d, 1H, J=3.6 Hz). MS (ES) [m+H] calc'd for C₁₁H₇F₃N₄S, 285. found 285.

### Example 2A

### (5-Trifluoromethyl-1H-indazol-3-yl)-thiourea

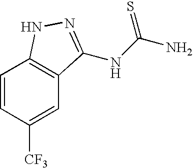

1,1′-Thiocarbonyldi-2(1H)-pyridone (578 mg, 2.49 mmol) was added to a solution of 5-trifluoromethyl-1H-indazol-3-ylamine (500 mg, 2.49 mmol) in CH₂Cl₂ (10 mL), and the reaction stirred for 2 h at r.t. The solution was added to a mixture of aq. NH₄OH (5 mL) in MeOH (5 mL), and the reaction stirred for 15 min. Organics were extracted with CHCl₃ (2×), dried (MgSO₄), and concentrated in vacuo to give 642 mg (99% yield) of the title compound as an off-white solid. ¹H NMR (400 MHz, DMSO-d₆): δ 8.29 (s, 1H), 7.57 (d, 1H, J=8.8 Hz), 7.48 (d, 1H, J=8.8 Hz). MS (ES) [m+H] calc'd for C₈H₆F₃N₃, 202. found 202.

### Example 2

### (4-Phenyl-thiazol-2-yl)-(5-trifluoromethyl-1H-indazol-3-yl)-amine

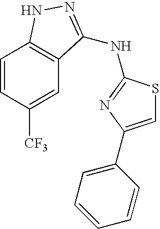

2-Bromoacetophenone (125 mg, 0.63 mmol) was added to a solution of (5-trifluoromethyl-1H-indazol-3-yl)-thiourea (160 mg, 0.61 mmol) in EtOH (6 mL), and the reaction stirred at reflux for 1 h. The solution was cooled, washed with brine, dried (MgSO₄), and concentrated in vacuo. Purification by silica gel chromatography (1:1:1 EtOAc/hexanes/CH₂Cl₂) gave 192 mg (87%) of the title compound as a white solid. ¹H NMR (400 MHz, DMSO-d6): δ 12.82 (s, 1H), 11.69 (s, 1H), 8.70 (s, 1H), 7.92 (d, 2H, J=7.6 Hz), 7.57-7.64 (m, 2H), 7.38-7.45 (m, 3H), 7.28-7.33 (m, 1H). MS (ES) [m+H] calc'd for C₁₇H₁₁F₃N₄S, 361. found 361.

### Example 3

### 2-(5-Trifluoromethyl-1H-indazol-3-ylamino)-thiazole-4-carboxylic acid ethyl ester

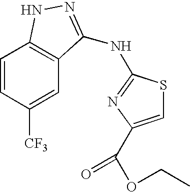

The title compound was prepared in 70% yield using ethyl bromopyruvate and a procedure analogous to that outlined in Example 2. ¹H NMR (400 MHz, DMSO-d6): δ 12.86 (s, 1H), 12.14 (s, 1H), 8.60 (s, 1H), 7.90 (s, 1H), 7.58-7.65 (m, 2H), 4.27 (q, 2H, J=7.2 Hz), 1.30 (t, 3H, J=7.2 Hz). MS (ES) [m+H] calc'd for C₁₄H₁₁F₃N₄O₂S, 357. found 357.

### Example 4

### (4-Phenyl-thiazol-2-yl)-(6-trifluoromethyl-1H-indazol-3-yl)-amine

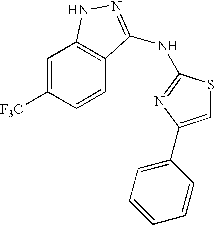

The title compound was prepared in 77% yield from 6-trifluoromethyl-1H-indazol-3-ylamine according to a procedure analogous to that outlined in Example 2. ¹H NMR (400 MHz, DMSO-d6): δ 12.82 (s, 1H), 11.69 (s, 1H), 8.36 (d, 1H, J=8.4 Hz), 7.92 (d, 2H, J=7.6 Hz), 7.79 (s, 1H), 7.28-7.47 (m, 5H). MS (ES) [m+H] calc'd for C₁₇H₁₁F₃N₄S, 361. found 361.

### Example 5

### 4-methyl-N-(5-(trifluoromethyl)-1H-indazol-3-yl)thiazol-2-amine

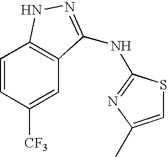

The title compound was prepared in 75% yield using α-bromoacetone according to a procedure analogous to that outlined in Example 2. ¹H NMR (400 MHz, MeOH-d4): δ 8.36 (s, 1H), 7.61-7-72 (m, 2H), 6.76 (s, 1H), 2.40 (s, 3H). MS (ES) [m+H] calc'd for C₁₂H₉F₃N₄S, 299. found 299.

### Example 6

### N-(1-benzyl-5-(trifluoromethyl)-1H-indazol-3-yl)thiazol-2-amine

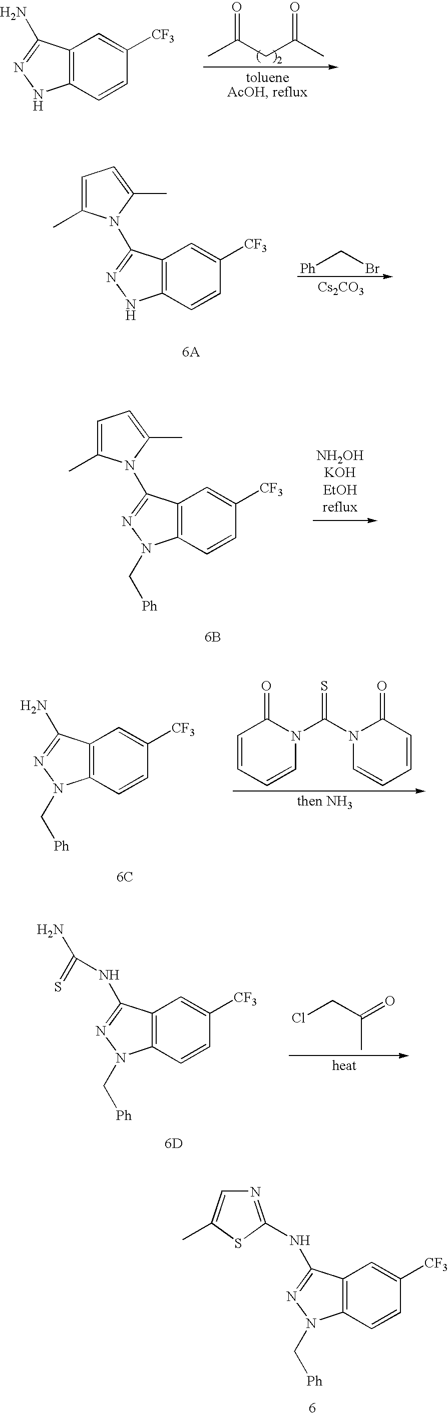

To a mixture of 5-trifluoromethyl-1H-indazol-3-ylamine (0.51 g, 2.54 mmol) and 2,5-hexandione (0.30 g, 2.6 mmol) in toluene (15 mL) was added 1 mL of AcOH. After refluxing for 24 hrs, the reaction was cooled, washed with water and dried with MgSO₄. The solvent was removed to give crude compound 6A, which was used for the next step without further purification.

A mixture of the crude product 6A, benzylbromide and Cs₂CO₃ in NMP (5 mL) was stirred at 60° C. overnight. The mixture was diluted with water, extracted with EtOAc and dried over MgSO₄. The solvent was removed in vacuo to give compound 6B. MS (ES) [m+H] calc'd for C₂₁18F₃N₃, 370. found 370.

A mixture of compound 6B, NH₂OH.HCl and KOH in EtOH was refluxed for 48 hours. LC/MS showed that the conversion was about 60%. The mixture was cooled to r.t., extracted with DCM and dried with MgSO₄. The solvent was removed to give crude compound 6C, which was used for the next step without further purification.

Compound 6D was prepared according to a procedure analogous to that described in connection with Example 2A. MS (ES) [m+H] calc'd for C₁₆H₁₃F₃N₄S, 351. found 351.

Compound 6 was prepared from compound 6D according to a procedure analogous to that described in connection with Example 2. ¹H NMR (400 MHz, DMSO-d6): δ 8.63 (s, 1H), 7.87 (d, 1H, J=8.0 Hz), 7.67 (d, 1H, J=8.0 Hz), 7.14-7.41 (m, 6H), 5.59 (s, 2H). MS (ES) [m+H] calc'd for C₁₆H₁₃F₃N₄S, 375. found 375.

### Example 7

### 5-Bromo-N-1,3-thiazol-2-yl-1H-indazol-3-amine

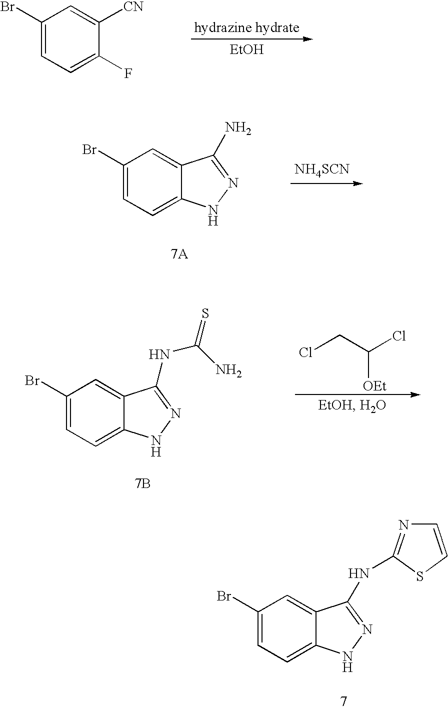

Hydrazine monohydrate (2.6 mL, 52.5 mmol) was added to a solution of 5-bromo-2-fluorobenzonitrile (3.50 g, 17.5 mmol) in ethanol (50 mL) at room temperature. The mixture was stirred for 4 h under reflux condition. After cooling, the mixture was diluted with EtOAc (300 mL), washed with H₂O and brine, dried (MgSO₄), filtered, and concentrated in vacuo. Purification by recrystallization (EtOAc-hexane) gave 3.37 g (91%) of 5-bromo-1H-indazol-3-amine (compound 7A) as a white solid. ¹H NMR (300 MHz, CDCl₃) δ 4.07 (brs, 2H) 7.20 (d, 1H, J=8.85 Hz) 7.42 (dd, 1H, J=8.85, 1.70 Hz) 7.71 (d, 1H, J=1.51 Hz) 8.98 (brs, 1H). MS (ES) [m+H] calc'd for C₇H₆BrN₃, 213. found 211, 213.

Ammonium thiocyanate (229 mg, 3 mmol) was added to a suspension of 5-bromo-1H-indazol-3-amine (212 mg, 1 mmol) in 1N hydrochloric acid (3 mL). The mixture was stirred for 4 days at 100° C. The precipitate was collected, and washed with H₂O to give 231 mg (85%) of N-(5-bromo-1H-indazol-3-yl)thiourea (compound 7B) as a yellow solid. ¹H NMR (300 MHz, DMSO-d₆) δ 7.43 (d, 1H, J=8.85 Hz) 7.50 (dd, 1H, J=8.85, 1.88 Hz) 8.49 (d, 1H, J=1.32 Hz) 8.79 (brs, 1H) 9.18 (brs, 1H) 10.85 (s, 1H) 12.86 (s, 1H). MS (ES) [m+H] calc'd for C₈H₇BrN₄S, 272. found 270, 272.

To a stirred solution of N-(5-bromo-1H-indazol-3-yl)thiourea (104 mg, 0.38 mmol) in ethanol (2 mL) and H₂O (1 mL) was added 1,2-dichloroethyl ethyl ether (0.05 mL, 0.41 mmol) at room temperature. The mixture was stirred for 3 h at 80° C. After dilution with EtOAc, the organic layer washed with H₂O and brine, dried (MgSO₄), filtered, and concentrated in vacuo. Crystallization from EtOAc-diisopropyl ether gave 62.7 mg (55%) of 5-Bromo-N-1,3-thiazol-2-yl-1H-indazol-3-amine (compound 7) as a white solid. ¹H NMR (300 MHz, DMSO-d₆) δ 7.01 (d, 1H, J=3.58 Hz) 7.36 (d, 1H, J=3.58 Hz) 7.37-7.42 (m, 1H) 7.44-7.49 (m, 1H) 8.35 (d, 1H, J=1.32 Hz) 11.34 (brs, 1H) 12.53 (s, 1H). MS (ES) [m+H] calc'd for C₁₀H₇BrN₄S, 296. found 294, 296.

### Example 8

### 5-Chloro-N-1,3-thiazol-2-yl-1H-indazol-3-amine

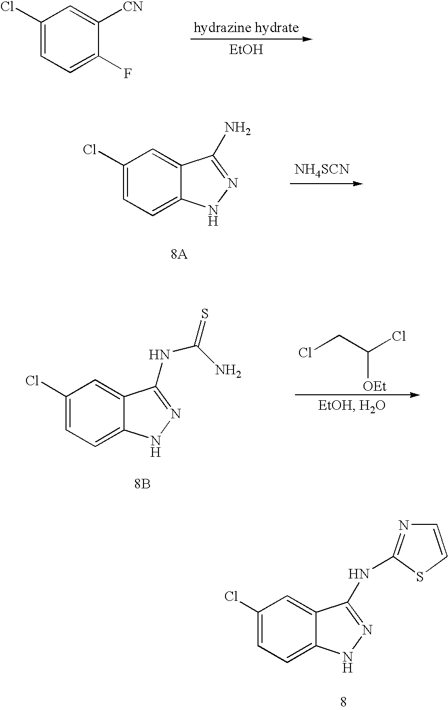

5-Chloro-1H-indazol-3-amine (compound 8A) was prepared in 63% yield from 5-chloro-2-fluorobenzonitrile according to a procedure analogous to that outlined in Example 7. ¹H NMR (300 MHz, CDCl₃) δ 4.07 (brs, 2H) 7.23-7.33 (m, 2H) 7.55 (d, 1H, J=1.88 Hz) 8.94 (brs, 1H).

Ammonium thiocyanate (1.54 g, 20.2 mmol) was added to a suspension of 5-chloro-1H-indazol-3-amine (564 mg, 3.37 mmol) in 1N hydrochloric acid (120 mL). The mixture was stirred for 4 days at 100° C. The precipitate was collected, washed with H₂O, and purified by recrystallization (EtOAc) to give 102 mg (13%) of N-(5-Chloro-1H-indazol-3-yl)thiourea (compound 8B) as a yellow solid. ¹H NMR (300 MHz, DMSO-d₆) δ 7.39 (dd, 1H, J=8.85, 2.07 Hz) 7.50 (dd, 1H, J=9.04, 0.57 Hz) 8.33 (d, 1H, J=1.51 Hz) 8.78 (brs, 1H) 9.19 (brs, 1H) 10.85 (s, 1H) 12.85 (s, 1H).

5-Chloro-N-1,3-thiazol-2-yl-1H-indazol-3-amine (compound 8) was prepared in 34% yield from N-(5-chloro-1H-indazol-3-yl)thiourea according to a procedure analogous to that outlined in Example 7. ¹H NMR (300 MHz, DMSO-d₆) δ 7.01 (d, 1H, J=3.58 Hz) 7.33-7.39 (m, 2H) 7.45 (d, 1H, J=9.04 Hz) 8.19 (d, 1H, J=1.51 Hz) 11.35 (brs, 1H) 12.53 (s, 1H). MS (ES) [m+] calc'd for C₁₀H₇ClN₄S, 250. found 250.

### Example 9

### 1-Methyl-N-1,3-thiazol-2-yl-5-(trifluoromethyl)-1H-indazol-3-amine

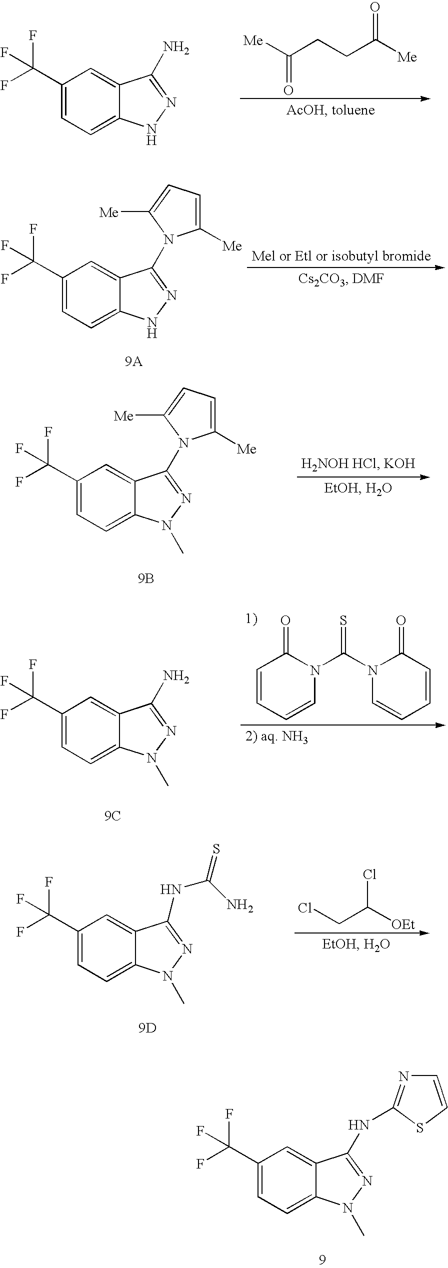

To a stirred solution of 5-trifluoromethyl-1H-indazol-3-amine (2.5 g, 12.4 mmol) in toluene (20 mL) were added acetic acid (0.15 mL, 2.48 mmol) and 2,5-hexandione (1.6 mL, 2.6 mmol) at room temperature. The mixture was stirred under reflux condition for 4 h while water was removed in a Dean-Stark apparatus. Additional acetic acid (0.15 mL, 2.48 mmol) and 2,5-hexandione (1.6 mL, 2.6 mmol) were added to the mixture, and the mixture was stirred under reflux condition overnight while water was removed in a Dean-Stark apparatus. After dilution with EtOAc (100 mL), the mixture washed with saturated aqueous NaHCO₃, H₂O, and brine. The organic layer was dried (MgSO₄), filtered, and concentrated in vacuo. Purification by silica gel chromatography (hexane/EtOAc=10:1 to 5:1) gave 3.15 g (91%) of 3-(2,5-Dimethyl-1H-pyrrol-1-yl)-5-(trifluoromethyl)-1H-indazole (compound (9A) as an amorphous powder. ¹H NMR (300 MHz, CDCl₃) δ 2.09 (s, 6H) 6.07 (s, 2H) 7.34 (d, 1H, J=8.85 Hz) 7.65 (dd, 1H, J=8.95, 1.60 Hz) 7.84 (s, 1H) 12.02 (brs, 1H). MS (ES) [m+H] calc'd for C₁₄H₁₂F₃N₃, 280. found 280.

To a stirred solution of 3-(2,5-dimethyl-1H-pyrrol-1-yl)-5-(trifluoromethyl)-1H-indazole (303 mg, 1.08 mmol) in DMF (5 mL) were added cesium carbonate (0.54 g, 1.62 mmol) and iodomethane (0.075 mL, 1.19 mmol) at room temperature. The mixture was stirred for 5 h at 60° C. After dilution with EtOAc, the organic layer washed with H₂O and brine, dried (MgSO₄), filtered, and concentrated in vacuo. The crude oil was passed through a pad of SiO₂ (eluent: EtOAc) and concentrated in vacuo to give 305 mg (96%) of t3-(2,5-Dimethyl-1H-pyrrol-1-yl)-1-methyl-5-(trifluoromethyl)-1H-indazole (compound 9B) as a white solid. ¹H NMR (300 MHz, CDCl₃) δ 2.05 (s, 6H) 4.15 (s, 3H) 5.97 (s, 2H) 7.55 (d, 1H, J=8.85 Hz) 7.66 (dd, 1H, J=9.04, 1.70 Hz) 7.80 (s, 1H). MS (ES) [m+H] calc'd for C₁₅H₁₄F₃N₃, 294. found 294.

To a stirred solution of 3-(2,5-dimethyl-1H-pyrrol-1-yl)-1-methyl-5-(trifluoromethyl)-1H-indazole (300 mg, 1.02 mmol) in ethanol (3 mL) and H₂O (1 mL) were added potassium hydroxide (172 mg, 3.06 mmol) and hydroxylamine hydrochloride (355 mg, 5.1 mmol) at room temperature. The mixture was stirred at 100° C. overnight. After dilution with H₂O, the aqueous layer was extracted with CH₂Cl₂ (30 mL×2). The combined organic layer was dried (MgSO₄), filtered, and concentrated in vacuo. Purification by silica gel chromatography (EtOAc/hexane=3:1 to 1:1) gave 100 mg (46%) of 1-Methyl-5-(trifluoromethyl)-1H-indazol-3-amine (compound 9C) as a white solid. ¹H NMR (300 MHz, CDCl₃) δ 3.88 (s, 3H) 4.12 (d, 2H, J=6.97 Hz) 7.24-7.30 (m, 1H) 7.54 (dd, 1H, J=8.85, 1.51 Hz) 7.85 (s, 1H).

To a stirred solution of 1-methyl-5-(trifluoromethyl)-1H-indazol-3-amine (100 mg, 0.465 mmol) in CH₂Cl₂ (5 mL) was added 1,1′-thiocarbonyldi-2(1H)-pyridone (119 mg, 0.511 mmol) at 0° C., and the mixture stirred for 2 h at 0° C. Aqueaous ammonia (28%, 5 mL) was added to the mixture, and the reaction mixture was stirred for 1 h at room temperature. After dilution with EtOAc, the organic layer was separated, washed with H₂O and brine, dried (MgSO₄), filtered, and concentrated in vacuo to give 129 mg (100% yield) of N-[1-Methyl-5-(trifluoromethyl)-1H-indazol-3-yl]thiourea (compound 9D) as a white solid. ¹H NMR (300 MHz, CDCl₃) δ 4.01 (s, 3H) 6.81 (brs, 1H) 7.43 (d, 1H, J=8.85 Hz) 7.67 (dd, 1H, J=8.95, 1.60 Hz) 7.98 (s, 1H) 8.37 (brs, 1H) 9.42 (brs, 1H). MS (ES) [m+H] calc'd for C₁₂H₉F₃N₄S, 275. found 275.

1-Methyl-N-1,3-thiazol-2-yl-5-(trifluoromethyl)-1H-indazol-3-amine (compound 9) was prepared in 39% yield from N-[1-methyl-5-(trifluoromethyl)-1H-indazol-3-yl]thiourea according to a procedure analogous to that outlined in Example 7. ¹H NMR (300 MHz, DMSO-d₆) δ 4.00 (s, 3H) 7.05 (d, 1H, J=3.58 Hz) 7.39 (d, 1H, J=3.58 Hz) 7.65-7.77 (m, 2H) 8.64 (s, 1H) 11.56 (s, 1H). MS (ES) [m+H] calc'd for C₁₂H₉F₃N₄S, 299. found 299.

### Example 10

### 1-Ethyl-N-1,3-thiazol-2-yl-5-(trifluoromethyl)-1H-indazol-3-amine

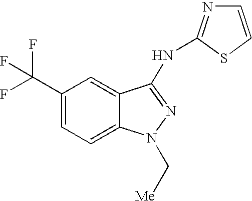

3-(2,5-Dimethyl-1H-pyrrol-1-yl)-1-ethyl-5-(trifluoromethyl)-1H-indazole was prepared in 96% yield from 3-(2,5-dimethyl-1H-pyrrol-1-yl)-5-(trifluoromethyl)-1H-indazole according to a procedure analogous to that outlined in Example 9. ¹H NMR (300 MHz, CDCl₃) δ 1.57 (t, 3H, J=7.25 Hz) 2.05 (s, 6H) 4.49 (q, 2H, J=7.16 Hz) 5.97 (s, 2H) 7.56 (d, 1H, J=8.85 Hz) 7.64 (dd, 1H, J=8.85, 1.51 Hz) 7.80 (s, 1H). MS (ES) [m+H] calc'd for C₁₆H₁₆F₃N₃, 308. found 308.

1-Ethyl-5-(trifluoromethyl)-1H-indazol-3-amine was prepared in 30% yield from 3-(2,5-dimethyl-1H-pyrrol-1-yl)-1-ethyl-5-(trifluoromethyl)-1H-indazole according to a procedure analogous to that outlined in Example 9. ¹H NMR (300 MHz, CDCl₃) δ 1.44 (t, 3H, J=7.25 Hz) 4.13 (brs, 2H) 4.23 (q, 2H, J=7.28 Hz) 7.29 (d, 1H, J=8.85 Hz) 7.53 (dd, 1H, J=8.85, 1.51 Hz) 7.85 (s, 1H)

N-[1-Ethyl-5-(trifluoromethyl)-1H-indazol-3-yl]thiourea was prepared in 97% yield from 1-ethyl-5-(trifluoromethyl)-1H-indazol-3-amine according to a procedure analogous to that outlined in Example 9. ¹H NMR (300 MHz, CDCl₃) δ 1.50 (t, 3H, J=7.25 Hz) 4.35 (q, 2H, J=7.16 Hz) 6.91 (brs, 1H) 7.44 (d, 1H, J=9.04 Hz) 7.66 (dd, 1H, J=9.04, 1.51 Hz) 8.04 (s, 1H) 8.60 (brs, 1H) 9.48 (brs, 1H). MS (ES) [m+H] calc'd for C₁₁H₁₁F₃N₄S, 289. found 289.

Compound 10 was prepared in 41% yield from N-[1-ethyl-5-(trifluoromethyl)-1H-indazol-3-yl]thiourea according to a procedure a procedure analogous to that outlined in Example 9. ¹H NMR (300 MHz, DMSO-d₆) δ 1.43 (t, 3H, J=7.16 Hz) 4.39 (q, 2H, J=7.16 Hz) 7.04 (d, 1H, J=3.58 Hz) 7.39 (d, 1H, J=3.77 Hz) 7.67 (dd, 1H, J=9.04, 1.51 Hz) 7.78 (d, 1H, J=8.85 Hz) 8.63 (s, 1H) 11.57 (s, 1H). MS (ES) [m+H] calc'd for C₁₃H₁₁F₃N₄S, 313. found 313.

### Example 11

### 1-Isobutyl-N-1,3-thiazol-2-yl-5-(trifluoromethyl)-1H-indazol-3-amine

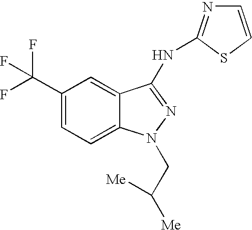

3-(2,5-Dimethyl-1H-pyrrol-1-yl)-1-isobutyl-5-(trifluoromethyl)-1H-indazole was prepared in 99% yield from 3-(2,5-dimethyl-1H-pyrrol-1-yl)-5-(trifluoromethyl)-1H-indazole according to a procedure analogous to that outlined in Example 9. ¹H NMR (300 MHz, CDCl₃) δ 0.96 (d, 6H, J=6.78 Hz) 2.04 (s, 6H) 2.32-2.50 (m, 1H) 4.24 (d, 2H, J=7.16 Hz) 5.97 (s, 2H) 7.55 (d, 1H, J=9.04 Hz) 7.63 (dd, 1H, J=7.54, 1.51 Hz) 7.80 (s, 1H). MS (ES) [m+H] calc'd for C₁₈H₂₀F₃N₃, 336. found 336.

1-Isobutyl-5-(trifluoromethyl)-1H-indazol-3-amine was prepared in 20% yield from 3-(2,5-dimethyl-1H-pyrrol-1-yl)-1-isobutyl-5-(trifluoromethyl)-1H-indazole according to a procedure analogous to that outlined in Example 9. ¹H NMR (300 MHz, CDCl₃) δ 0.91 (d, 6H, J=6.78 Hz) 2.19-2.35 (m, 1H) 3.96 (d, 2H, J=7.35 Hz) 4.11 (brs, 2H) 7.24-7.31 (m, 1H) 7.51 (dd, 1H, J=8.95, 1.60 Hz) 7.84 (s, 1H).

N-[1-Isobutyl-5-(trifluoromethyl)-1H-indazol-3-yl]thiourea was prepared in 100% yield from 1-isobutyl-5-(trifluoromethyl)-1H-indazol-3-amine according to a procedure analogous to that outlined in Example 9. ¹H NMR (300 MHz, CDCl₃) δ 0.93 (d, 6H, J=6.78 Hz) 2.18-2.37 (m, 1H) 4.08 (d, 2H, J=7.35 Hz) 6.87 (brs, 1H) 7.42 (d, 1H, J=9.04 Hz) 7.64 (dd, 1H, J=9.04, 1.51 Hz) 8.01 (s, 1H) 8.52 (brs, 1H) 9.47 (brs, 1H). MS (ES) [m+H] calc'd for C₁₃H₁₅F₃N₄S, 317. found 317.

Compound 11 was prepared in 30% yield from N-[1-isobutyl-5-(trifluoromethyl)-1H-indazol-3-yl]thiourea according to a procedure a procedure analogous to that outlined in Example 7. ¹H NMR (300 MHz, DMSO-d₆) δ 0.89 (d, 6H, J=6.78 Hz) 2.23-2.39 (m, 1H) 4.18 (d, 2H, J=6.97 Hz) 7.04 (d, 1H, J=3.58 Hz) 7.38 (d, 1H, J=3.58 Hz) 7.66 (dd, 1H, J=8.95, 1.60 Hz) 7.79 (d, 1H, J=9.04 Hz) 8.63 (s, 1H) 11.55 (brs, 1H). MS (ES) [m+H] calc'd for C₁₅H₁₅F₃N₄S, 341. found 341.

### Example 12

### tert-Butyl {3-[3-(1,3-thiazol-2-ylamino)-5-(trifluoromethyl)-1H-indazol-1-yl]propyl}carbamate

### Example 13

### 1-(3-Aminopropyl)-N-1,3-thiazol-2-yl-5-(trifluoromethyl)-1H-indazol-3-amine

### Example 14

### 1-[3-(Dimethylamino)propyl]-N-1,3-thiazol-2-yl-5-(trifluoromethyl)-1H-indazol-3-amine

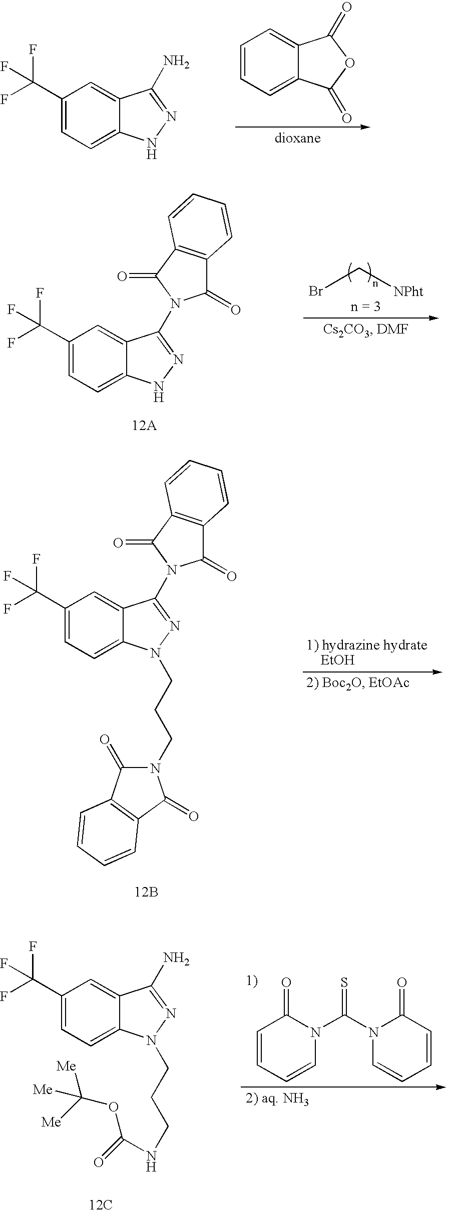

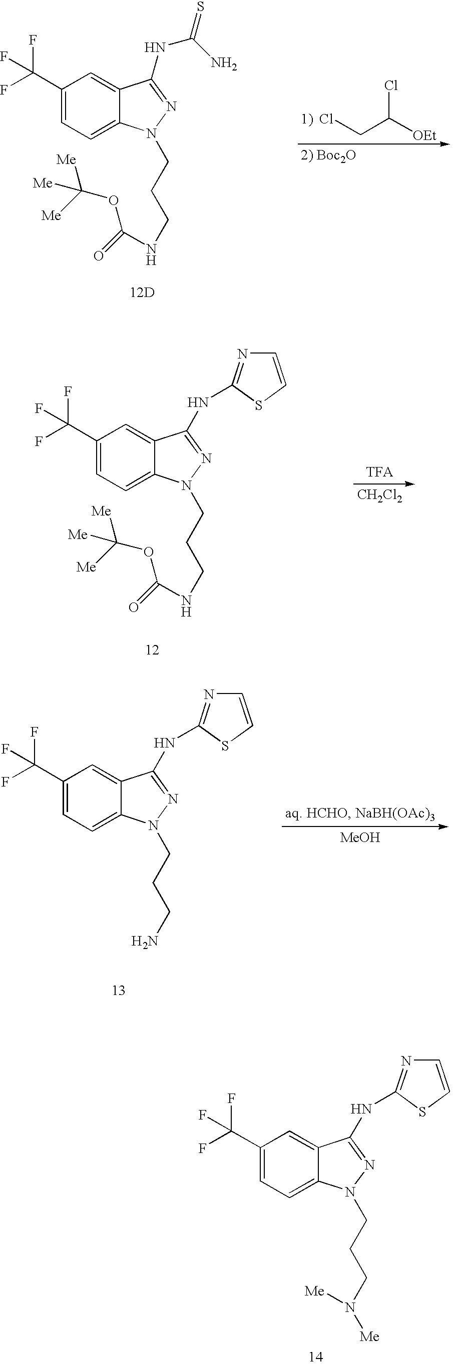

Phthalic anhydride (2.59 g, 17.5 mmol) was added to a solution of 5-trifluoromethyl-1H-indazol-3-amine (2.516 g, 12.5 mmol) in 1,4-dioxane (100 mL) at room temperature. The mixture was stirred at 120° C. overnight. After evaporation of the solvent, diethyl ether was added, and the mixture was stirred for 30 min at room temperature. The precipitate was collected, and washed with diethyl ether to give 2.26 g (56%) of compound 12A as a white solid. The filtrate was concentrated in vacuo. The solid was collected, and washed with diethyl ether to give 242 mg (5.8%) of the title compound as a white solid. ¹H NMR (300 MHz, DMSO-d₆) δ 7.71 (dd, 1H, J=9.04, 1.51 Hz) 7.80-7.88 (m, 1H) 7.94-8.00 (m, 2H) 8.00-8.07 (m, 2H) 8.34-8.42 (m, 1H) 13.88 (brs, 1H). MS (ES) [m+H] calc'd for C₁₆H₈F₃N₃O₂, 332. found 332.

To a stirred solution of 2-[5-(trifluoromethyl)-1H-indazol-3-yl]-1H-isoindole-1,3(2H)-dione (500 mg, 1.51 mmol) in DMF (10 mL) were added N-(3-bromopropyl)phthalimide (446 mg, 1.66 mmol) and cesium carbonate (0.74 g, 2.27 mmol) at room temperature. The mixture was stirred at 100° C. overnight. After cooling, H₂O was added to the mixture. The aqueous layer was extracted with EtOAc, and the organic layer washed with H₂O and brine, dried (MgSO₄), filtered, and concentrated in vacuo. Purification by silica gel chromatography (EtOAc/hexanes=10:1) gave 210 mg (27%) of compound 12B as a white solid. ¹H NMR (300 MHz, CDCl₃) δ 2.37-2.54 (m, 2H) 3.87 (t, 2H, J=6.59 Hz) 4.49-4.63 (m, 2H) 7.57-7.68 (m, 2H) 7.68-7.73 (m, 2H) 7.81-7.89 (m, 5H) 8.02 (dd, 2H, J=5.56, 3.11 Hz). MS (ES) [m+H] calc'd for C₂₇H₁₇F₃N₄O₄, 519. found 519.

Hydrazine monohydrate (0.12 mL, 2.43 mmol) was added to a stirred solution of 2-[1-[3-(1,3-dioxo-1,3-dihydro-2H-isoindol-2-yl)propyl]-5-(trifluoromethyl)-1H-indazol-3-yl]-1H-isoindole-1,3(2H)-dione (210 mg, 0.405 mmol) in ethanol (10 mL). The mixture was stirred for 3 h under reflux condition. After cooling, saturated aqueous NaHCO₃ and di-tert-butyl dicarbonate (100 mg, 0.458 mmol) were added to the mixture, and the mixture was stirred at room temperature overnight. The reaction mixture was diluted with EtOAc, the organic layer washed with H₂O and brine, dried (MgSO₄), filtered, and concentrated in vacuo. Purification by silica gel chromatography gave 126 mg (87%) of compound 12C as a colorless oil. ¹H NMR (300 MHz, CDCl₃) δ 1.42 (s, 9H) 1.94-2.15 (m, 2H) 2.98-3.21 (m, 2H) 4.13 (s, 2H) 4.24 (t, 2H, J=6.63 Hz) 4.75 (brs, 1H) 7.28 (d, 1H, J=8.71 Hz) 7.53 (dd, 1H, J=9.09, 1.51 Hz) 7.85 (s, 1H). MS (ES) [m-Boc+2H] calc'd for C₁₁H₁₃F₃N₄, 259. found 259.

Compound 12D was prepared in 80% yield from tert-butyl {3-[3-amino-5-(trifluoromethyl)-1H-indazol-1-yl]propyl}carbamate according to a procedure analogous to that outlined in Example 9. ¹H NMR (300 MHz, CDCl₃) δ 1.43 (s, 9H) 2.06-2.16 (m, 2H) 3.18 (s, 2H) 4.35 (t, 2H, J=6.63 Hz) 4.58 (brs, 1H) 6.94 (brs, 1H) 7.43 (d, 1H, J=9.09 Hz) 7.65 (d, 1H, J=9.09 Hz) 8.09 (s, 1H) 8.82 (brs, 1H) 9.45 (brs, 1H). MS (ES) [m+H] calc'd for C₁₇H₂₂F₃N₅O₂S, 418. found 418.

To a stirred solution of tert-butyl {3-[3-[(aminocarbonothioyl)amino]-5-(trifluoromethyl)-1H-indazol-1-yl]propyl}carbamate (118 mg, 0.283 mmol) in ethanol (3 mL) and H₂O (1 mL) was added 1,2-dichloroethyl ethyl ether (0.11 mL, 0.848 mmol) at room temperature. The mixture was stirred for 4 h at 90° C. After cooling, saturated aqueous NaHCO₃, EtOAc and di-tert-butyl dicarbonate (93 mg, 0.43 mmol) were added to the mixture, and the mixture was stirred at room temperature overnight. The reaction mixture was diluted with EtOAc, the organic layer washed with H₂O and brine, dried (MgSO₄), filtered, and concentrated in vacuo. Purification by silica gel chromatography gave 76.1 mg (61%) of compound 12 as a colorless oil. ¹H NMR (300 MHz, CDCl₃) δ 1.43 (s, 9H) 2.12-2.22 (m, 2H) 3.14-3.22 (m, 2H) 4.41 (t, 2H, J=6.44 Hz) 4.92 (brs, 1H) 6.87 (d, 1H, J=3.79 Hz) 7.41 (s, 1H) 7.39 (d, 1H, J=6.06 Hz) 7.62 (d, 1H, J=8.71 Hz) 8.00 (s, 1H) 9.94 (brs, 1H). MS (ES) [m+H] calc'd for C₁₉H₂₂F₃N₅O₂S, 442. found 442.

Trifluoroacetic acid (2 mL) was added to a stirred solution of tert-butyl {3-[3-(1,3-thiazol-2-ylamino)-5-(trifluoromethyl)-1H-indazol-1-yl]propyl}carbamate (68.4 mg, 0.155 mmol) in CH₂Cl₂ (3 mL). The mixture was stirred for 1 h at room temperature. Saturated aqueous NaHCO₃ was added to the mixture. The aqueous layer was extracted with EtOAc. The organic layer was dried (MgSO₄), filtered, and concentrated in vacuo to give 50.2 mg (95%) of compound 13 as a white powder. ¹H NMR (300 MHz, DMSO-d₆) δ 1.99-2.12 (m, 2H) 2.71-2.80 (m, 2H) 4.44 (t, 2H, J=6.59 Hz) 7.07 (d, 1H, J=3.58 Hz) 7.40 (d, 1H, J=3.58 Hz) 7.68-7.73 (m, 1H) 7.80 (d, 1H, J=9.04 Hz) 8.64 (s, 1H). MS (ES) [m+H] calc'd for C₁₄H₁₄F₃N₅S, 342. found 342.

To a stirred solution of 1-(3-aminopropyl)-N-1,3-thiazol-2-yl-5-(trifluoromethyl)-1H-indazol-3-amine (41.7 mg, 0.122 mmol) in MeOH (2 mL) was added formalin (30 mg, 0.366 mmol) at room temperature. After the mixture was stirred for 30 min at room temperature, sodium triacetoxyhydroborate (78 mg, 0.366 mmol) was added, and the mixture was stirred for 4 h at room temperature. The reaction was quenched with adding saturated aqueous NaHCO₃. The aqueous layer was extracted with EtOAc. The organic layer was dried (MgSO₄), filtered, and concentrated in vacuo. The crude oil was purified by silica gel chromatography (NH-SiO₂, EtOAc/hexane=5:1 to 1:1), followed by crystallization with diisopropyl ether to give 18.3 mg (41%) of the compound 14 as a white powder. ¹H NMR (300 MHz, DMSO-d₆) δ 2.00 (t, 2H, J=6.50 Hz) 2.10 (s, 6H) 2.13-2.21 (m, 2H) 4.37 (t, 2H, J=6.50 Hz) 7.05 (d, 1H, J=3.39 Hz) 7.39 (d, 1H, J=3.58 Hz) 7.67 (d, 1H, J=9.23 Hz 7.74 (d, 1H, J=8.85 Hz) 8.62 (s, 1H) 11.58 (brs, 1H). MS (ES) [m+H] calc'd for C₁₆H₁₈F₃N₅S, 370. found 370.

### Example 15

### tert-Butyl {4-[3-(1,3-thiazol-2-ylamino)-5-(trifluoromethyl)-1H-indazol-1-yl]butyl}carbamate

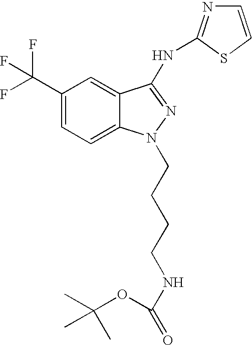

2-[1-[4-(1,3-Dioxo-1,3-dihydro-2H-isoindol-2-yl)butyl]-5-(trifluoromethyl)-1H-indazol-3-yl]-1H-isoindole-1,3(2H)-dione was prepared in 50% yield from 2-[5-(trifluoromethyl)-1H-indazol-3-yl]-1H-isoindole-1,3(2H)-dione according to a procedure analogous to that outlined in Example 12 (compound 12B, where n=4). ¹H NMR (300 MHz, CDCl₃) δ 1.70-1.89 (m, 2H) 1.93-2.14 (m, 2H) 3.77 (t, 2H, J=6.78 Hz) 4.54 (t, 2H, J=7.06 Hz) 7.59-7.66 (m, 2H) 7.71 (dd, 2H, J=5.46, 3.01 Hz) 7.80-7.91 (m, 5H) 8.02 (dd, 2H, J=5.46, 3.01 Hz). MS (ES) [m+H] calc'd for C28H19F3N4O4, 533. found 533.

tert-Butyl {4-[3-amino-5-(trifluoromethyl)-1H-indazol-1-yl]butyl}carbamate was prepared in 41% yield from 2-[1-[4-(1,3-dioxo-1,3-dihydro-2H-isoindol-2-yl)butyl]-5-(trifluoromethyl)-1H-indazol-3-yl]-1H-isoindole-1,3(2H)-dione according to a procedure analogous to that outlined in Example 12 (compound 12C, where n=4). ¹H NMR (300 MHz, CDCl₃) δ 1.43 (s, 9H) 1.48 (s, 2H) 1.83-1.97 (m, 2H) 3.03-3.24 (m, 2H) 4.11 (brs, 2H) 4.20 (t, 2H, J=7.00 Hz) 4.53 (brs, 1H) 7.31 (d, 1H, J=8.71 Hz) 7.52 (dd, 1H, J=9.09, 1.51 Hz) 7.84 (s, 1H). MS (ES) [m-Boc+2H] calc'd for C₁₂H₁₅F₃N₄, 273. found 273.

tert-Butyl {4-[3-[(aminocarbonothioyl)amino]-5-(trifluoromethyl)-1H-indazol-1-yl]butyl}carbamate was prepared in 94% yield from tert-butyl {4-[3-amino-5-(trifluoromethyl)-1H-indazol-1-yl]butyl}carbamate according to a procedure analogous to that outlined in Example 9. ¹H NMR (300 MHz, CDCl₃) δ 1.43 (s, 9H) 1.46-1.54 (m, 2H) 1.87-2.00 (m, 2H) 3.17 (q, 2H, J=7.07 Hz) 4.34 (t, 2H, J=7.00 Hz) 4.53 (brs, 1H) 6.82 (brs, 1H) 7.48 (d, 1H, J=8.71 Hz) 7.65 (dd, 1H, J=8.90, 1.33 Hz) 7.98 (s, 1H) 8.41 (brs, 1H) 9.41 (brs, 1H). MS (ES) [m+H] calc'd for C₁₈H₂₄F₃N₅O₂S, 432. found 432.

Compound 15 was prepared in 64% yield from tert-butyl {4-[3-[(aminocarbonothioyl)amino]-5-(trifluoromethyl)-1H-indazol-1-yl]butyl}carbamate according to a procedure analogous to that outlined in Example 12 (compound 12, where n=4). ¹H NMR (300 MHz, CDCl₃) δ 1.43 (s, 9H) 1.49-1.64 (m, 2H) 2.01 (brs, 2H) 3.14-3.25 (m, 2H) 4.35 (t, J=6.63 Hz, 2H) 4.53 (brs, 1H) 6.86 (brs, 1H) 7.42 (d, 2H, J=8.71 Hz) 7.60 (d, 1H, J=9.09 Hz) 8.01 (brs, 1H) 10.04 (brs, 1H). MS (ES) [m-Boc+2H] calc'd for C₁₅H₁₆F₃N₅S, 356. found 356.

### Example 16

### 1-(4-Aminobutyl)-N-1,3-thiazol-2-yl-5-(trifluoromethyl)-1H-indazol-3-amine

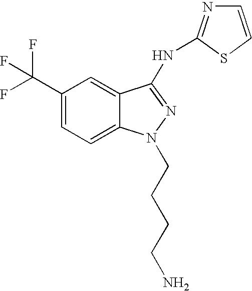

The title compound was prepared in 99% yield from tert-butyl {4-[3-(1,3-thiazol-2-ylamino)-5-(trifluoromethyl)-1H-indazol-1-yl]butyl}carbamate according to a procedure analogous to that outlined in Example 13. ¹H NMR (300 MHz, DMSO-d₆) δ 1.34-1.60 (m, 2H) 1.82-2.05 (m, 2H) 2.76 (t, 2H, J=7.76 Hz) 4.40 (t, 2H, J=6.63 Hz) 7.05 (d, 1H, J=3.41 Hz) 7.40 (d, 1H, J=3.79 Hz) 7.69 (dd, 1H, J=9.09, 1.51 Hz) 7.80 (d, 1H, J=8.71 Hz) 8.64 (s, 1H). MS (ES) [m+H] calc'd for C₁₅H₁₆F₃N₅S, 356. found 356.

### Example 17

### 1-[4-(Dimethylamino)butyl]-N-1,3-thiazol-2-yl-5-(trifluoromethyl)-1H-indazol-3-amine

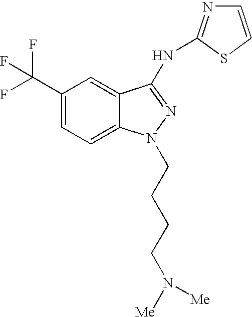

The title compound was prepared in 11% yield from 1-(4-aminobutyl)-N-1,3-thiazol-2-yl-5-(trifluoromethyl)-1H-indazol-3-amine according to a procedure analogous to that outlined in Example 14. ¹H NMR (300 MHz, DMSO-d₆) δ 1.37 (qd, 2H, J=7.44, 7.25 Hz) 1.81-1.94 (m, 2H) 2.05 (s, 6H) 2.19 (t, 2H, J=7.25 Hz) 4.36 (t, 2H, J=6.69 Hz) 6.55 (s, 1H) 7.05 (d, 1H, J=3.58 Hz) 7.39 (d, 1H, J=3.58 Hz) 7.64-7.70 (m, 1H) 7.78 (d, 1H, J=8.85 Hz) 8.63 (s, 1H). MS (ES) [m+H] calc'd for C₁₇H₂₀F₃N₅S, 384. found 384.

### Example 18

### [3-(1,3-Thiazol-2-ylamino)-1H-indazol-5-yl]methanol

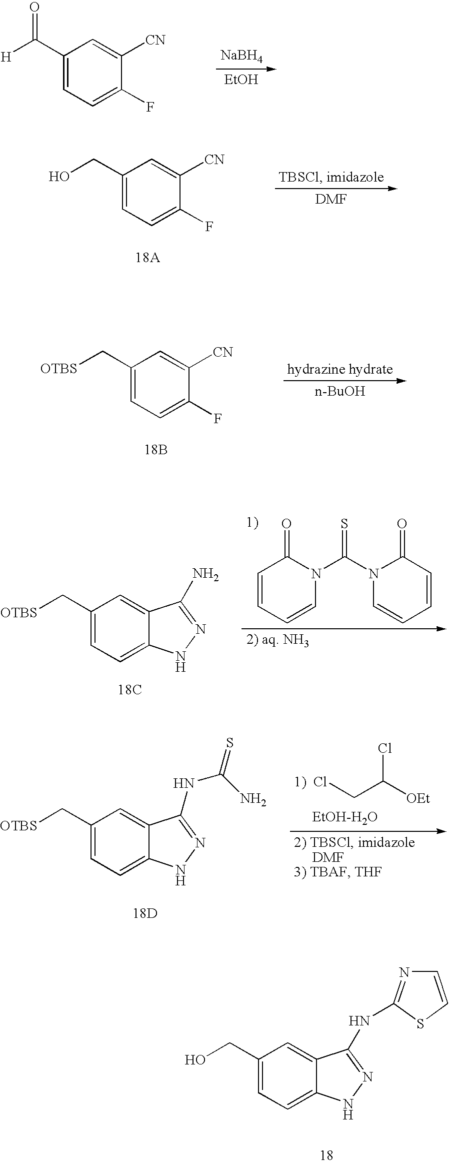

Sodium borohydride (1.32 g, 34.9 mmol) was added to a solution of 2-fluoro-5-formylbenzonitrile (4.73 g, 31.7 mmol) at 0° C. The mixture was stirred for 1 h at room temperature, quenched with 1N HCl. The mixture was extracted with EtOAc, and the organic layer washed with H₂O and brine, dried (MgSO₄), filtered, and concentrated in vacuo to give 4.726 g (99%) of 2-fluoro-5-(hydroxymethyl)benzonitrile (compound 18A) as a white solid. ¹H NMR (300 MHz, CDCl₃) δ 1.89 (brs, 1H) 4.73 (s, 2H) 7.21 (t, 1H, J=8.67 Hz) 7.56-7.69 (m, 2H).

To a stirred solution of 2-fluoro-5-(hydroxymethyl)benzonitrile in DMF (60 mL) were added imidazole (4.90 g, 31.3 mmol) and tert-butyldimethylchlorosilane (5.19 g, 34.4 mmol) at room temperature. The mixture was stirred at room temperature for 2 h. After dilution with EtOAc, the organic layer washed with H₂O and brine, dried (MgSO₄), filtered, and concentrated in vacuo to give crude 5-({[tert-butyl(dimethyl)silyl]oxy}methyl)-1H-indazol-3-amine (compound 18B), which was used for the next step without further purification.

Hydrazine monohydrate (4.6 mL, 93.9 mmol) was added to a solution of the above crude compound 18B (31.3 mmol) in n-BuOH (100 mL) at room temperature. The mixture was stirred for overnight under reflux condition. After cooling, the precipitate was filtered off, and the filtrate was concentrated in vacuo. The crude oil was purified by silica gel chromatography (EtOAc/hexane=10:1) to give 1.003 g (12%) of compound 18C as a brown oil. ¹H NMR (300 MHz, CDCl₃) δ 0.11 (s, 6H) 0.95 (s, 9H) 4.09 (brs, 2H) 4.81 (s, 2H) 7.23-7.37 (m, 2H) 7.51 (s, 1H) 8.92 (brs, 1H). MS (ES) [m+H] calc'd for C₁₅H₂₄N₄OSSi, 278. found 278.

N-[5-({[tert-Butyl(dimethyl)silyl]oxy}methyl)-1H-indazol-3-yl]thiourea (compound 18D) was prepared in 78% yield from 5-({[tert-butyl(dimethyl)silyl]oxy}methyl)-1H-indazol-3-amine according to a procedure analogous to that outlined in Example 9. ¹H NMR (300 MHz, CDCl₃) δ 0.14 (s, 6H) 0.96 (s, 9H) 4.83 (s, 2H) 6.81 (brs, 1H) 7.35-7.48 (m, 2H) 7.58 (s, 1H) 8.29 (brs, 1H) 9.39 (brs, 1H) 9.57 (brs, 1H). MS (ES) [m+H] calc'd for C15H24N4OSSi, 337. found 337.

To a stirred solution of N-[5-({[tert-butyl(dimethyl)silyl]oxy}methyl)-1H-indazol-3-yl]thiourea (940 mg, 2.79 mmol) in ethanol (30 mL) and H₂O (10 mL) was added 1,2-dichloroethyl ethyl ether (1.03 mL, 8.38 mmol) at room temperature. The mixture was stirred for 3 h at 80° C. After dilution with EtOAc, the organic layer washed with saturated aqueous NaHCO₃, H₂O and brine, dried (MgSO₄), filtered, and concentrated in vacuo. Crystallization from diisopropyl ether gave 584 mg of the crude mixture as a white solid which was used for the next step without further purification. To a stirred solution of the crude compound in DMF (10 mL) was added imidazole (380 mg, 5.45 mmol) and tert-butyldimethylchlorosilane (400 mg, 2.61 mmol) at room temperature. The mixture was stirred overnight at room temperature, diluted with EtOAc, washed with H₂O and brine, dried (MgSO₄), filtered, and concentrated in vacuo. Purification by silica gel chromatography gave 237 mg of the crude compound as a colorless oil. To a stirred solution of the crude oil (237 mg) in THF (10 mL) was added tetra-n-butylammonium fluoride (1M in THF, 3 mL, 3 mmol) at 0° C. The mixture was stirred for 2 h at room temperature. After dilution with EtOAc, the organic layer washed with H₂O and brine, dried (MgSO₄), filtered, and concentrated in vacuo. Purification by silica gel chromatography gave 25.5 mg (6.8%) of [3-(1,3-thiazol-2-ylamino)-1H-indazol-5-yl]methanol (compound 18) as a white solid. ¹H NMR (300 MHz, DMSO-d₆) δ 4.55 (d, 2H, J=5.46 Hz) 5.14 (t, 1H, J=5.65 Hz) 6.97 (d, 1H, J=3.58 Hz) 7.30-7.38 (m, 3H) 8.02 (s, 1H) 11.26 (s, 1H) 12.25 (s, 1H)

### Example 19

### 5-Ethyl-N-1,3-thiazol-2-yl-1H-indazol-3-amine

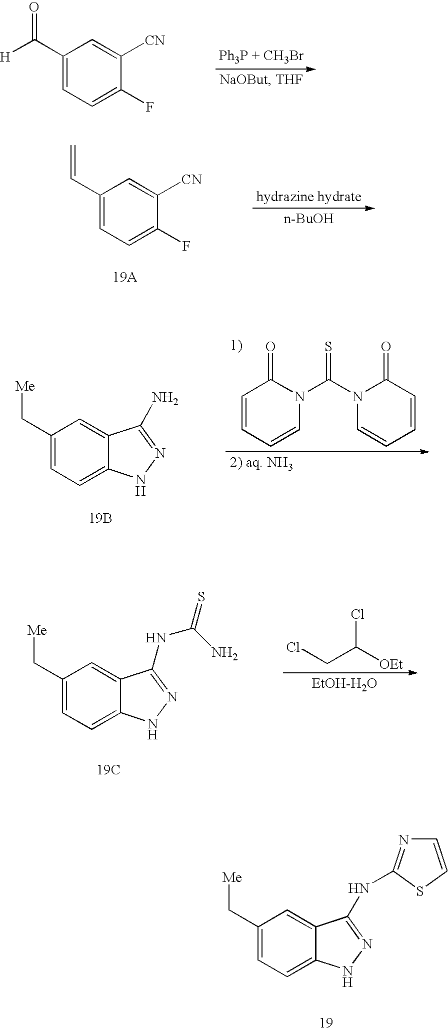

To a stirred suspension of methyltriphenylphosphonium bromide (6.80 g, 17.3 mmol) in THF (100 mL) was added potassium tert-butoxide (2.50 g, 26.0 mmol) at 0° C. The mixture was stirred for 30 min at 0° C. 2-Fluoro-5-formylbenzonitrile (2.58 g, 17.3 mmol) in THF (50 mL) was added dropwise to the reaction mixture at 0° C. The mixture was stirred for 4 h at room temperature. To the mixture was added 1N HCl, and the aqueous layer was extracted with EtOAc. The organic layer washed with H₂O and brine, dried (MgSO₄), filtered, and concentrated in vacuo. Purification by silica gel chromatography (EtOAc/hexane=1:10) gave 0.81 g (32%) of 2-fluoro-5-vinylbenzonitrile (compound 19A) as a colorless oil. ¹H NMR (300 MHz, CDCl₃) δ 5.33 (dd, 1H, J=26.56, 10.93 Hz) 5.72 (dd, 1H, J=17.52, 16.20 Hz) 6.57-6.71 (m, 1H) 7.07-7.22 (m, 1H) 7.49-7.65 (m, 2H). MS (ES) [m+H] calc'd for C₉H₆FN, 148. found 148.

5-Ethyl-1H-indazol-3-amine (compound 19) was prepared in 20% yield from 2-fluoro-5-vinylbenzonitrile according to a procedure analogous to that outlined in Example 7. ¹H NMR (300 MHz, CDCl₃) δ 1.28 (t, 3H, J=7.57 Hz) 2.74 (q, 2H, J=7.57 Hz) 4.05 (brs, 2H) 7.23 (s, 2H) 7.35 (s, 1H) 8.76 (brs, 1H). MS (ES) [m+H] calc'd for C₉H₁₁N₃, 162. found 162.

N-(5-Ethyl-1H-indazol-3-yl)thiourea (compound 19C) was prepared in 96% yield from 5-ethyl-1H-indazol-3-amine according to a procedure analogous to that outlined in Example 9. ¹H NMR (300 MHz, CDCl₃) δ 1.29 (t, 3H, J=7.57 Hz) 2.77 (q, 2H, J=7.57 Hz) 6.91 (brs, 1H) 7.27 (s, 1H) 7.34 (s, 1H) 7.46 (s, 1H) 8.52 (brs, 1H) 9.53 (brs, 2H). MS (ES) [m+H] calc'd for C₁₀H₁₂N₄S, 221. found 221.

5-Ethyl-N-1,3-thiazol-2-yl-1H-indazol-3-amine (compound 19) was prepared in 32% yield from N-(5-ethyl-1H-indazol-3-yl)thiourea according to a procedure analogous to that outlined in Example 7. ¹H NMR (300 MHz, DMSO-d₆) δ 1.24 (t, 3H, J=7.54 Hz) 2.69 (q, 2H, J=7.54 Hz) 6.96 (d, 1H, J=3.58 Hz) 7.23 (dd, 1H, J=8.67, 1.61 Hz) 7.31 (d, 1H, J=8.67 Hz) 7.34 (d, 1H, J=3.58 Hz) 7.92 (s, 1H) 11.20 (brs, 1H) 12.18 (s, 1H). MS (ES) [m+H] calc'd for C₁₂H₁₂N₄S, 245. found 245.

### Example 20

### 5-Nitro-N-1,3-thiazol-2-yl-1H-indazol-3-amine

### Example 21

### N³-1,3-Thiazol-2-yl-1H-indazole-3,5-diamine

### Example 22

### N-[3-(1,3-Thiazol-2-ylamino)-1H-indazol-5-yl]acetamide

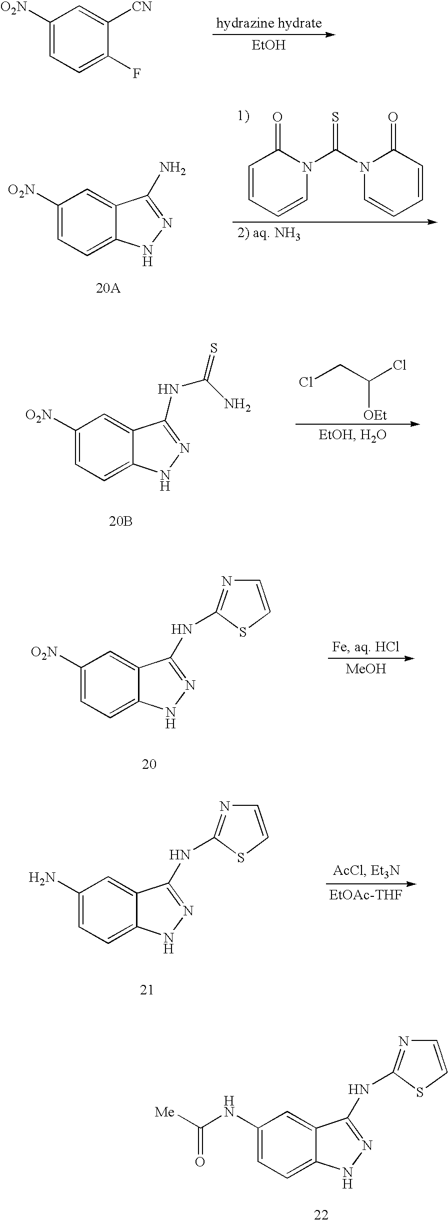

5-Nitro-1H-indazol-3-amine (compound 20A) was prepared in 100% yield from 5-bromo-2-fluorobenzonitrile according to a procedure analogous to that outlined in Example 7. ¹H NMR (300 MHz, DMSO-d₆) δ 5.99 (s, 2H) 7.35 (d, 1H, J=9.04 Hz) 8.06 (dd, 1H, J=9.23, 2.26 Hz) 8.90 (d, 1H, J=1.70 Hz) 12.18 (brs, 1H). MS (ES) [m+H] calc'd for C₇H₆N₄O₂, 179. found 179.

N-(5-Nitro-1H-indazol-3-yl)thiourea (compound 20B) was prepared in 28% yield from 5-nitro-1H-indazol-3-amine according to a procedure analogous to that outlined in Example 9. ¹H NMR (300 MHz, DMSO-d₆) δ 7.61 (d, 1H, J=9.23 Hz) 8.21 (dd, 1H, J=9.32, 2.17 Hz 8.96 (brs, 1H) 9.21 (brs, 1H) 9.47 (d, J=1.88 Hz, 1H) 11.25 (brs, 1H) 13.32 (brs, 1H). MS (ES) [m+H] calc'd for C₈H₇N₅O₂S, 238. found 238.

5-Nitro-N-1,3-thiazol-2-yl-1H-indazol-3-amine (compound 20) was prepared in 70% yield from N-(5-nitro-1H-indazol-3-yl)thiourea according to a procedure analogous to that outlined in Example 7. ¹H NMR (300 MHz, DMSO-d₆) δ 7.09 (d, 1H, J=3.79 Hz) 7.41 (d, 1H, J=3.41 Hz) 7.57 (d, 1H, J=9.47 Hz) 8.20 (dd, 1H, J=9.28, 2.08 Hz) 9.31 (d, 1H, J=1.51 Hz) 11.75 (brs, 1H) 13.06 (s, 1H). MS (ES) [m+H] calc'd for C₁₀H₇N₅O₂S, 262. found 262.

To a stirred suspension of 5-nitro-N-1,3-thiazol-2-yl-1H-indazol-3-amine (319 mg, 1.22 mmol) in MeOH (6 mL), H₂O (3 mL), and conc. HCl (0.3 mL) was added reduced iron (340 mg, 6.1 mmol) at room temperature. The mixture was stirred for 30 min at 80° C. After cooling, the insoluble material was filtered, and washed with MeOH. The filtrate was concentrated in vacuo. Saturated aqueous NaHCO₃ was added, and the mixture was extracted with THF-EtOAc. The combined organic layer washed with brine, dried (MgSO₄), filtered, and concentrated in vacuo. The residue in EtOAc was passed through a short SiO₂ column, and the filtrate was concentrated in vacuo. The residue was recrystallized from THF-diisopropyl ether, and washed with EtOAc to give 62.8 mg (22%) of N³-1,3-thiazol-2-yl-1H-indazole-3,5-diamine (compound 21) as a colorless solid. The filtrate was again concentrated in vacuo. The solid was collected, and washed with EtOAc to give 102 mg (36%) of compound 21 as a colorless solid. ¹H NMR (300 MHz, DMSO-d₆) δ 4.74 (brs, 2H) 6.80 (dd, 1H, J=8.76, 1.98 Hz) 6.90 (d, 1H, J=3.77 Hz) 7.06 (d, 1H, J=1.70 Hz) 7.13 (d, 1H, J=8.67 Hz) 7.29 (d, 1H, J=3.58 Hz) 10.94 (brs, 1H) 11.82 (s, 1H). MS (ES) [m+H] calc'd for C₁₀H₉N₅S, 232. found 232.

To a stirred solution of N³-1,3-thiazol-2-yl-1H-indazole-3,5-diamine (55.4 mg, 0.24 mmol) in EtOAc (2 mL) and THF (2 mL) were added triethylamine (0.041 mL, 0.288 mmol) and acetyl chloride (0.017 mL, 0.24 mmol) at 0° C. The mixture was stirred for 2 h at room temperature. After dilution with EtOAc, the organic layer washed with H₂O and brine, dried (MgSO₄), filtered, and concentrated in vacuo. Purification by recrystallization (EtOAc-diisopropyl ether) gave 17.2 mg (26%) of N-[3-(1,3-Thiazol-2-ylamino)-1H-indazol-5-yl]acetamide (compound 22) as a colorless solid. ¹H NMR (300 MHz, DMSO-d₆) δ 2.06 (s, 3H) 6.94 (d, 1H, J=3.03 Hz) 7.26-7.45 (m, 3H) 8.32 (s, 1H) 9.87 (s, 1H) 11.29 (brs, 1H) 12.23 (s, 1H).

### Example 23

### N-1,3-Thiazol-2-yl-7-(trifluoromethyl)-1H-indazol-3-amine

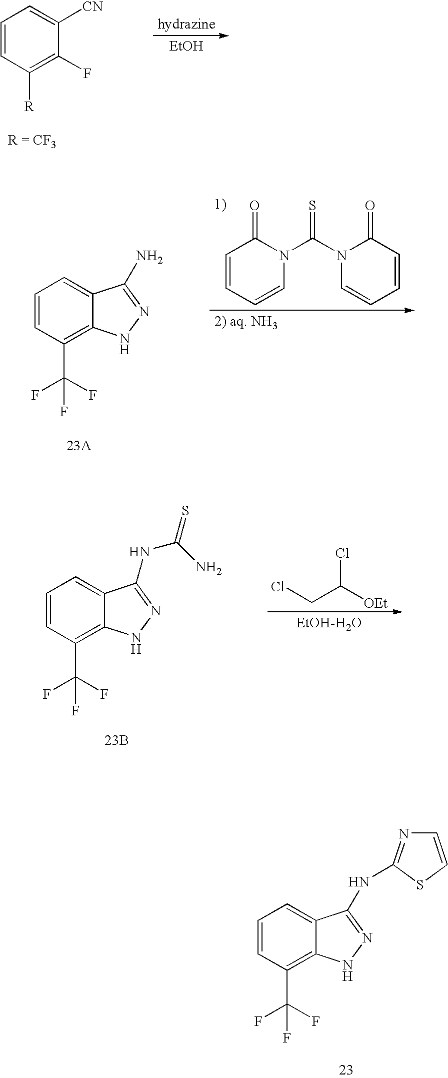

7-(Trifluoromethyl)-1H-indazol-3-amine (compound 23A) was prepared in 58% yield from 3-trifluoro-2-fluorobenzonitrile according to a procedure analogous to that outlined in Example 7. ¹H NMR (300 MHz, CDCl₃) δ 4.21 (brs, 2H) 7.15 (t, 1H, J=7.76 Hz) 7.62 (d, 1H, J=7.19 Hz) 7.76 (d, 1H, J=7.95 Hz) 9.31 (brs, 1H). MS (ES) [m+H] calc'd for C₈H₆F₃N₃, 202. found 202.

N-[7-(Trifluoromethyl)-1H-indazol-3-yl]thiourea (compound 23B) was prepared in 96% yield from 7-(trifluoromethyl)-1H-indazol-3-amine according to a procedure analogous to that outlined in Example 9. ¹H NMR (300 MHz, DMSO-d₆) δ 7.27 (t, 1H, J=7.76 Hz) 7.80 (d, 1H, J=7.19 Hz) 8.52 (d, 1H, J=8.33 Hz) 8.85 (brs, 1H) 9.16 (brs, 1H) 10.98 (brs, 1H) 13.20 (brs, 1H). MS (ES) [m+H] calc'd for C₉H₇F₃N₄S, 261. found 261.

N-1,3-Thiazol-2-yl-7-(trifluoromethyl)-1H-indazol-3-amine (compound 23) was prepared in 73% yield from N-[7-(trifluoromethyl)-1H-indazol-3-yl]thiourea according to a procedure analogous to that outlined in Example 7. ¹H NMR (300 MHz, DMSO-d₆) δ 7.05 (d, 1H, J=3.79 Hz) 7.23 (t, 1H, J=7.76 Hz) 7.39 (d, 1H, J=3.79 Hz) 7.76 (d, 1H, J=7.19 Hz) 8.43 (d, 1H, J=8.33 Hz) 11.56 (brs, 1H) 12.93 (brs, 1H). MS (ES) [m+H] calc'd for C₁₁H₇F₃N₄S, 285. found 285.

### Example 24

### 7-Fluoro-N-1,3-thiazol-2-yl-1H-indazol-3-amine

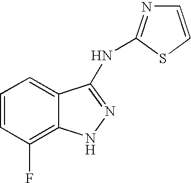

7-Fluoro-1H-indazol-3-amine was prepared in 73% yield from 2,3-difluorobenzonitrile according to a procedure analogous to that outlined in Example 7. ¹H NMR (300 MHz, CDCl₃) δ 4.15 (d, 2H, J=7.19 Hz) 6.95-7.09 (m, 2H) 7.34 (d, 1H, J=7.57 Hz) 9.19 (brs, 1H). MS (ES) [m+H] calc'd for C₇H₆FN₃, 152. found 152.

N-(7-Fluoro-1H-indazol-3-yl)thiourea was prepared in 75% yield from 7-fluoro-1H-indazol-3-amine according to a procedure analogous to that outlined in Example 9. ¹H NMR (300 MHz, DMSO-d₆) δ 7.06 (td, 1H, J=7.95, 4.54 Hz) 7.25 (dd, 1H, J=11.36, 7.19 Hz) 8.02 (d, 1H, J=8.33 Hz) 8.79 (brs, 1H) 9.16 (brs, 1H) 10.89 (s, 1H) 13.23 (brs, 1H). MS (ES) [m+H] calc'd for C₈H₇FN₄S, 211. found 211.

7-Fluoro-N-1,3-thiazol-2-yl-1H-indazol-3-amine (compound 24) was prepared in 57% yield from N-(7-fluoro-1H-indazol-3-yl)thiourea according to a procedure analogous to that outlined in Example 7. ¹H NMR (300 MHz, DMSO-d₆) δ 6.98-7.08 (m, 2H) 7.22 (dd, 1H, J=11.74, 7.57 Hz) 7.37 (d, 1H, J=3.41 Hz) 7.93 (d, 1H, J=8.33 Hz) 11.43 (brs, 1H) 12.85 (brs, 1H). MS (ES) [m+H] calc'd for C₁₀H₇FN₄S, 235. found 235.

### Example 25

### 1-[3-(1,3-Thiazol-2-ylamino)-1H-indazol-5-yl]ethanone

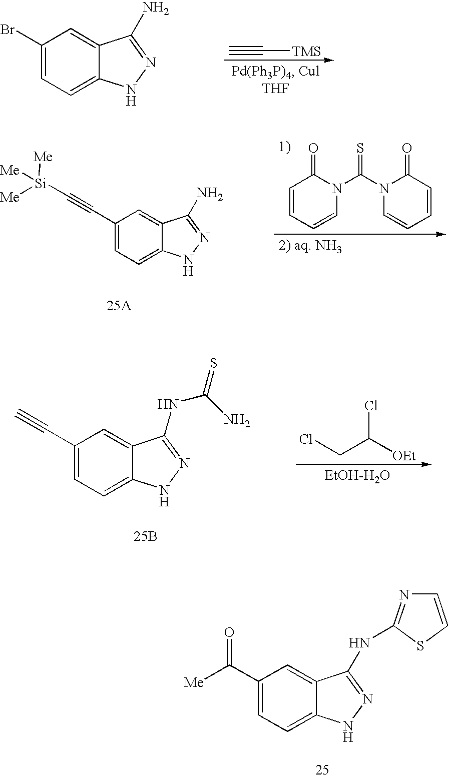

To a stirred solution of 5-bromo-1H-indazol-3-amine (215 mg, 1.01 mmol) in THF (10 mL) were added trimethylsilylacetylene (0.22 mL, 1.5 mmol), diisopropylethylamine (0.52 mL, 3.0 mmol), copper iodide (19 mg, 0.1 mmol), and dichlorobis(triphenylphosphine)palladium (71 mg, 0.1 mmol) at room temperature under nitrogen atmosphere. The mixture was stirred at 60° C. overnight. The insoluble material was filtered off, and the filtrate was diluted with EtOAc, washed with H₂O brine, dried (MgSO₄), filtered, and concentrated in vacuo. Purification by recrystallization (EtOAc-diisopropyl ether) gave 93.7 mg of a mixture of 5-[(trimethylsilyl)ethynyl]-1H-indazol-3-amine and 5-bromo-1H-indazol-3-amine.

1,1′-Thiocarbonyldi-2(1H)-pyridone (105 mg, 0.451 mmol) was added to a solution of the above mixture (93.7 mg) in CH₂Cl₂ (3 mL) at 0° C., and the reaction stirred for 2 h at 0° C. Aqueaous ammonia (28%, 5 mL) was added to the mixture, and the reaction mixture was stirred for 1 h at room temperature. After dilution with EtOAc, the organic layer was separated, washed with H₂O and brine, dried (MgSO₄), filtered, and concentrated in vacuo to give the crude thiourea (compound 25B) as a white solid which was used for the next step without further purification.

To a stirred solution of the crude thiourea in ethanol (4.5 mL) and H₂O (1.5 mL) was added 1,2-dichloroethyl ethyl ether (0.21 mL, 1.23 mmol) at room temperature. The mixture was stirred for 3 h at 80° C. After dilution with EtOAc, the organic layer washed with H₂O and brine, dried (MgSO₄), filtered, and concentrated in vacuo. Purification by silica gel chromatography (hexane:EtOAc=3:1) gave 2.1 mg (8% in 3 steps) of the title compound as a colorless oil. ¹H NMR (300 MHz, DMSO-d₆) δ 2.60 (s, 3H) 7.05 (d, 1H, J=3.58 Hz) 7.39 (d, 1H, J=3.58 Hz) 7.45 (d, 1H, J=8.85 Hz) 7.93 (dd, 1H, J=8.85, 1.51 Hz) 8.99 (s, 1H) 11.55 (brs, 1H) 12.71 (brs, 1H). MS (ES) [m+H] calc'd for C₁₂H₁₀N₄OS, 259. found 259.

### Example 26

### 5-Methoxy-N-1,3-thiazol-2-yl-1H-indazol-3-amine

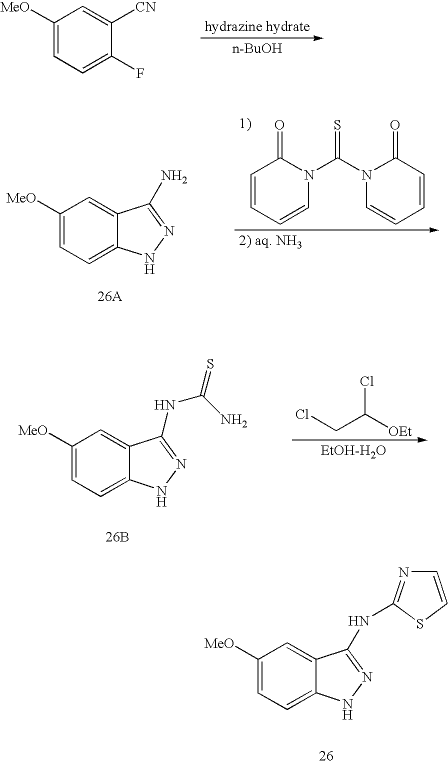

5-Methoxy-1H-indazol-3-amine (compound 26A) was prepared in 3.6% yield from 5-methoxy-2-fluorobenzonitrile according to a procedure analogous to that outlined in Example 7. ¹H NMR (300 MHz, CDCl₃) δ 3.85 (s, 3H) 3.99 (brs, 2H) 6.91 (d, 1H, J=2.27 Hz) 7.01-7.08 (m, 1H) 7.22 (d, 1H, J=9.09 Hz) 8.78 (brs, 1H). MS (ES) [m+H] calc'd for C₈H₉N₃O, 164. found 164.

5-Methoxy-1H-indazol-3-amine (compound 26B) was prepared in 53% yield from 5-methoxy-1H-indazol-3-amine according to a procedure analogous to that outlined in Example 9. ¹H NMR (300 MHz, DMSO-d₆) δ 3.77 (s, 3H) 7.04 (dd, 1H, J=9.04, 2.45 Hz) 7.35 (d, 1H, J=9.04 Hz) 7.75 (d, 1H, J=2.26 Hz) 8.67 (brs, 1H) 9.26 (brs, 1H) 10.73 (brs, 1H) 12.49 (brs, 1H). MS (ES) [m+H] calc'd for C₉H₁₀N₄OS, 223. found 223.

The title compound was prepared in 49% yield from N-(5-methoxy-1H-indazol-3-yl)thiourea according to a procedure analogous to that outlined in Example 7. ¹H NMR (300 MHz, DMSO-d₆) δ 3.78 (s, 3H) 6.96 (d, 1H, J=3.41 Hz) 7.02 (dd, 1H, J=8.90, 2.46 Hz) 7.28-7.36 (m, 2H) 7.62 (d, 1H, J=2.27 Hz) 11.14 (brs, 1H) 12.15 (s, 1H). MS (ES) [m+H] calc'd for C₁₁H₁₀N₄OS, 247. found 247.

### Example 27

### 5-Propyl-N-1,3-thiazol-2-yl-1H-indazol-3-amine

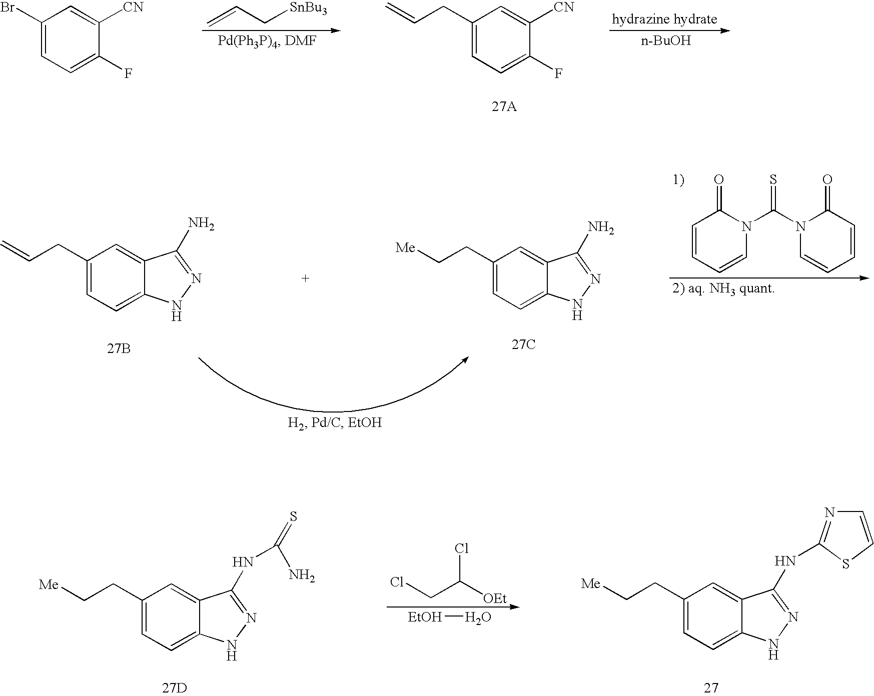

To a stirred solution of 5-bromo-2-fluorobenzonitrile (1.50 g, 7.50 mmol) in DMF (20 mL) were added allyl tributyltin (3.73 g, 11.25 mmol) and tetrakis(triphenylphosphine)palladium (433 mg, 0.375 mmol) under nitrogen atmosphere at room temperature. The mixture was stirred at 50° C. overnight. After cooling, the mixture was diluted with EtOAc, washed with 1N HCl, H₂O, and brine, dried (MgSO₄), filtered, and concentrated in vacuo. The crude oil was passed through a short NH-SiO₂ column, and the filtrate was concentrated in vacuo. Purification by silica gel chromatography (hexane/EtOAc=20:1 to 10:1) gave 739 mg (61%) of 5-allyl-2-fluorobenzonitrile (compound 27A) as a colorless oil. ¹H NMR (300 MHz, CDCl₃) δ 3.39 (d, 2H, J=6.59 Hz) 5.02-5.23 (m, 2H) 5.79-6.01 (m, 1H) 7.14 (t, 1H, J=8.57 Hz) 7.35-7.50 (m, 2H)

To a stirred solution of 5-allyl-2-fluorobenzonitrile (0.74 g, 4.57 mmol) in n-BuOH (30 mL) was added hydrazine monohydrate (0.67 mL, 13.8 mmol). The mixture was stirred under reflux overnight, and concentrated in vacuo. The residue was diluted with EtOAc, washed with H₂O and brine, dried (MgSO₄), filtered, and concentrated in vacuo. The precipitate was collected, and washed with hexane to give the mixture of 5-allyl-1H-indazol-3-amine (compound 27B) and 5-propyl-1H-indazol-3-amine (compound 27C) (2:5). The mixture was dissolved with ethanol (2 mL). Palladium charcoal (80 mg) was added to the solution, and the mixture was stirred for 4 h under hydrogen atmosphere at room temperature. The catalyst was removed by filtration and the filtrate was concentrated in vacuo to give 83.2 mg (10%) of compound 27C as a colorless solid. ¹H NMR (300 MHz, CDCl₃) δ 0.95 (t, 3H, J=7.25 Hz) 1.60-1.77 (m, 2H) 2.61-2.76 (m, 2H) 4.05 (brs, 2H) 7.16-7.23 (m, 2H) 7.33 (s, 1H) 8.75 (brs, 1H). MS (ES) [m+H] calc'd for C₁₀H₁₃N₃, 176. found 176.

N-(5-Propyl-1H-indazol-3-yl)thiourea (compound 27D) was prepared in 100% yield from 5-propyl-1H-indazol-3-amine according to a procedure analogous to that outlined in Example 9. ¹H NMR (300 MHz, CDCl₃) δ 0.96 (t, 3H, J=7.38 Hz) 1.61-1.77 (m, 2H) 2.71 (t, 2H, J=7.57 Hz) 6.81 (brs, 1H) 7.28-7.37 (m, 2H) 7.41 (s, 1H) 8.32 (brs, 1H) 9.36 (brs, 1H) 9.55 (brs, 1H). MS (ES) [m+H] calc'd for C₁₁H₁₄N₄S, 235. found 235.

The title compound was prepared in 29% yield from N-(5-propyl-1H-indazol-3-yl)thiourea according to a procedure analogous to that outlined in Example 7. ¹H NMR (300 MHz, DMSO-d₆) δ 0.92 (t, 3H, J=7.38 Hz) 1.55-1.72 (m, 2H) 2.63 (t, 2H, J=7.57 Hz) 6.96 (d, 1H, J=3.41 Hz) 7.21 (dd, 1H, J=8.33, 1.14 Hz) 7.31 (d, 1H, J=8.71 Hz) 7.34 (d, 1H, J=3.79 Hz) 7.89 (s, 1H) 11.18 (s, 1H) 12.18 (s, 1H). MS (ES) [m+H] calc'd for C₁₃H₁₄N₄S, 259. found 259.

### Example 28

### 3-(1,3-Thiazol-2-ylamino)-1H-indazol-5-ol

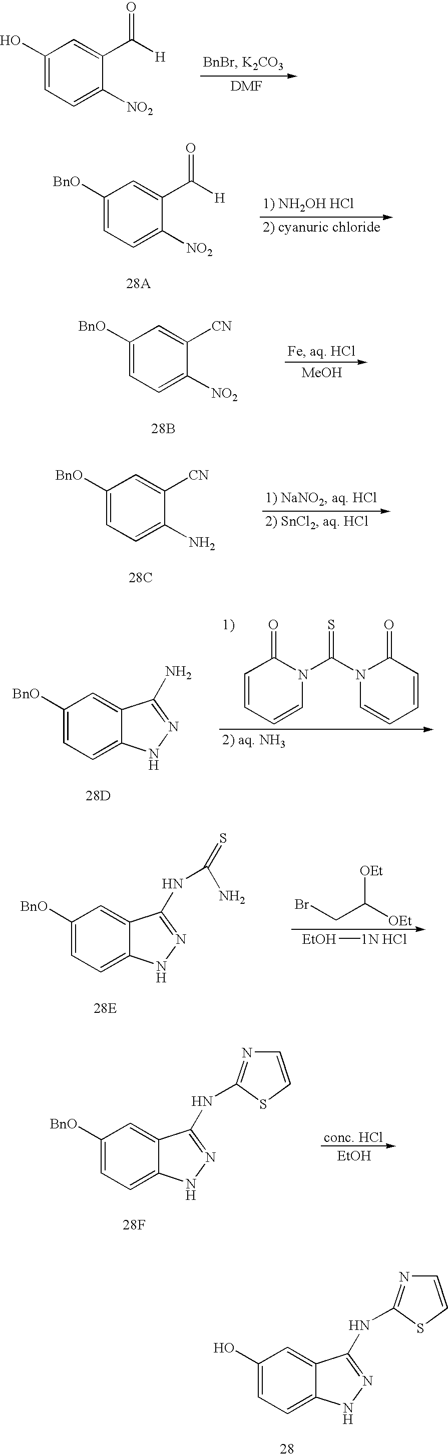

To a stirred solution of 5-hydroxy-2-nitrobenzaldehyde (25 g, 150 mmol) in DMF (200 mL) were added potassium carbonate (25 g, 180 mmol) and benzyl bromide (20 mL, 165 mmol) at room temperature and the mixture was stirred for 2 h at 80° C. After cooling, the mixture was diluted with EtOAc, washed with H₂O and brine, dried (MgSO₄), filtered, and concentrated in vacuo. Purification by recrystallization (hexane-EtOAc) gave 34.15 g (99%) of 5-(benzyloxy)-2-nitrobenzaldehyde (compound 28A) as a colorless solid. ¹H NMR (300 MHz, CDCl₃) δ 5.21 (s, 2H) 7.21 (dd, 1H, J=9.09, 3.03 Hz) 7.35-7.46 (m, 6H) 8.16 (d, 1H, J=9.09 Hz) 10.48 (s, 1H).

To a stirred solution of 5-(benzyloxy)-2-nitrobenzaldehyde (20 g, 86.9 mmol) in pyridine (100 mL) was added hydroxylamine hydrochloride (6.68 g, 96 mmol) at room temperature, and the mixture was stirred for 2 h at 80° C. and concentrated in vacuo. H₂O was added to the residue and the precipitate was collected, washed with H₂O, and dried to give the crude oxime which was used for the next step without further purification. To the crude oxime in DMF (200 mL) was added cyanuric chloride (16.03 g, 86.9 mmol) at 0° C. The mixture was stirred for 1 h at 0° C., and then for 1 h at room temperature. 1N HCl (200 mL) was added to the mixture at 0° C. The aqueous layer was extracted with EtOAc. The organic layer washed with H₂O and brine, dried (MgSO₄), filtered, and concentrated in vacuo. Purification by recrystallization (hexane-EtOAc) gave 17.83 g (81%) of 5-(benzyloxy)-2-nitrobenzonitrile (compound 28B) as a colorless solid. ¹H NMR (300 MHz, CDCl₃) δ 5.21 (s, 2H) 7.20-7.31 (m, 1H) 7.35-7.51 (m, 6H) 8.31 (d, 1H, J=9.23 Hz).

To a stirred solution of 5-isopropoxy-2-nitrobenzonitrile (17.83 g, 70.1 mmol) in MeOH (70 mL) and conc. HCl (70 mL) was added reduced iron (3.92 g, 70.1 mmol) at room temperature. The mixture was stirred at 60° C. for 2 h, and concentrated in vacuo. EtOAc, THF, and saturated aqueous NaHCO₃ were added to the residue and the insoluble material was removed by filtration. The organic layer washed with H₂O and brine, dried (MgSO₄), filtered, and concentrated in vacuo. The residue was passed through a pad of silica gel column (eluent: EtOAc). Purification by recrystallization from hexane-EtOAc gave 12.85 g (82%) of 2-Amino-5-(benzyloxy)benzonitrile (compound 28C) as a light yellow solid. ¹H NMR (300 MHz, CDCl₃) δ 4.12 (brs, 2H) 4.98 (s, 2H) 6.69 (d, 1H, J=8.71 Hz) 6.94 (d, 1H, J=2.65 Hz) 6.98-7.11 (m, 1H) 7.30-7.47 (m, 5H). MS (ES) [m+H] calc'd for C₁₄H₁₂N₂O, 225. found 225.

To a stirred solution of 2-amino-5-(benzyloxy)benzonitrile (5.0 g, 22.3 mmol) in conc. HCl (40 mL) was added sodium nitrite (1.93 mg, 27.9 mmol) in H₂O (10 mL) at 0° C., and the mixture was stirred for 30 min. The resulting solution was added dropwise to stannous chloride (34.0 g, 178 mmol) in conc. HCl (20 ml) at 0° C. The mixture was stirred for 2 h at room temperature, and concentrated in vacuo. To the residue were added 8N NaOH, EtOAc, and THF, and the insoluble material was removed by filtration. The organic layer was separated, washed with H₂O and brine, dried (MgSO₄), filtered, and concentrated in vacuo. The precipitate was collected, and recrystallized from hexane-EtOAc to give 3.334 g (62%) of 5-(benzyloxy)-1H-indazol-3-amine (compound 28D) as a colorless solid. ¹H NMR (300 MHz, DMSO-d₆) δ 5.05 (s, 2H) 5.17 (s, 2H) 6.97 (dd, 1H, J=8.85, 2.45 Hz) 7.15 (d, 1H, J=9.04 Hz) 7.25-7.44 (m, 4H) 7.44-7.53 (m, 2H) 11.20 (s, 1H). MS (ES) [m+H] calc'd for C₁₄H₁₃N₃O, 240. found 240.

N-[5-(Benzyloxy)-1H-indazol-3-yl]thiourea (compound 28E) was prepared in 100% yield from 5-(benzyloxy)-1H-indazol-3-amine according to a procedure analogous to that outlined in Example 9. ¹H NMR (300 MHz, DMSO-d₆) δ 5.08 (s, 2H) 7.12 (dd, 1H, J=9.04, 2.26 Hz) 7.30-7.46 (m, 4H) 7.46-7.54 (m, 2H) 7.89 (d, 1H, J=1.88 Hz) 8.69 (d, 1H, J=1.32 Hz) 9.27 (brs, 1H) 10.74 (brs, 1H) 12.52 (brs, 1H). MS (ES) [m+H] calc'd for C₁₅H₁₄N₄OS, 299. found 299.

To a stirred solution of N-[5-(benzyloxy)-1H-indazol-3-yl]thiourea (1.953 g, 6.27 mmol) in ethanol (45 mL) and H₂O (15 mL) was added bromoacetaldehyde diethylacetal (1.54 mL, 12.54 mmol) at room temperature. The mixture was stirred for 4 h at 80° C. After dilution with EtOAc, the organic layer washed with saturated aqueous NaHCO₃, H₂O and brine, dried (MgSO₄), filtered, and concentrated in vacuo. The residue was dissolved with EtOAc, passed through SiO₂ pad (NH-SiO₂, EtOAc), and concentrated in vacuo. Crystallization from EtOAc-diisopropyl ether gave 2.51 g (100%) of 5-(Benzyloxy)-N-1,3-thiazol-2-yl-1H-indazol-3-amine (compound 28F) as a white solid. ¹H NMR (300 MHz, DMSO-d₆) δ 5.09 (s, 2H) 6.96 (d, 1H, J=3.58 Hz) 7.10 (dd, 1H, J=8.95, 2.35 Hz) 7.29-7.45 (m, 5H) 7.46-7.54 (m, 2H) 7.73 (d, 1H, J=2.26 Hz) 11.15 (brs, 1H) 12.17 (s, 1H).

A solution of 5-(benzyloxy)-N-1,3-thiazol-2-yl-1H-indazol-3-amine (1.65 g, 5.19 mmol) in ethanol (30 mL) and conc. HCl (30 mL) was stirred at 100° C. overnight. The mixture was concentrated in vacuo. The resulting solid was collected, washed with H₂O and dried, and recrystallized from THF-diisopropyl ether to give 435 mg (36%) of the title compound as a colorless solid. ¹H NMR (300 MHz, DMSO-d₆) δ 6.88-6.97 (m, 2H) 7.22 (d, 1H, J=8.85 Hz) 7.28-7.38 (m, 2H) 9.05 (s, 1H) 11.08 (brs, 1H) 12.00 (s, 1H). MS (ES) [m+H] calc'd for C₁₀H₈N₄OS, 233. found 233.

### Example 29

### 5-Isobutyl-N-1,3-thiazol-2-yl-1H-indazol-3-amine

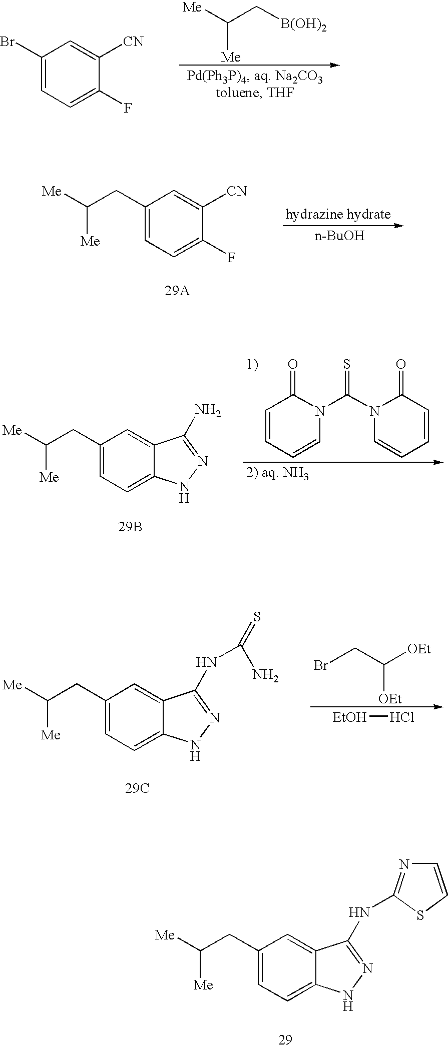

To a stirred solution of 5-bromo-2-fluorobenzonitrile (1.00 g, 5.0 mmol) in toluene (12 mL) and THF (8 ml) were added potassium carbonate (2.07 g, 15 mmol), isobutyl boronic acid (0.77 g, 7.5 mmol), and tetrakis(triphenylphosphine)palladium (289 mg, 0.25 mmol) under nitrogen atmosphere at room temperature. The mixture was stirred at 80° C. overnight. After cooling, the mixture was diluted with EtOAc, washed with H₂O and brine, dried (MgSO₄), filtered, and concentrated in vacuo. The crude oil was passed through a short SiO₂ pad (NH-SiO₂, EtOAc), and the elution fraction was concentrated in vacuo to give crude product (compound 29A), which was used for the next step without further purification.

To a stirred solution of the crude product of compound 29A in n-BuOH (20 mL) was added hydrazine monohydrate (0.73 mL, 15 mmol) at room temperature. The mixture was stirred under reflux condition overnight, and concentrated in vacuo. The mixture was diluted with EtOAc-THF, washed with H₂O and brine, dried (MgSO₄), filtered, and concentrated in vacuo to give the crude product of compound 29B, which was used for the next step without further purification.

To a solution of the crude product of compound 29B in THF (20 mL) was added 1,1′-thiocarbonyldi-2(1H)-pyridone (920 mg, 3.95 mmol) at 0° C., and the reaction was stirred for 1 h at 0° C. Aqueous ammonia (28%, 5 mL) was added to the mixture, and the reaction mixture was stirred for 1 h at room temperature. After dilution with EtOAc, the organic layer washed with H₂O and brine, dried (MgSO₄), filtered, and concentrated in vacuo. The crude product (N-(5-isobutyl-1H-indazol-3-yl)thiourea; compound 29C) was passed through a short SiO₂ pad (EtOAc), and concentrated in vacuo. Prification by preparative HPLC gave 97.4 mg (7.8% in 3 steps) of compound 29C as a colorless solid. ¹H NMR (300 MHz, DMSO-d₆) δ 0.88 (d, 6H, J=6.44 Hz) 1.75-1.95 (m, 1H) 2.52 (d, 2H, J=3.79 Hz) 7.21 (dd, 1H, J=8.71, 1.51 Hz) 7.34 (d, 1H, J=8.71 Hz) 7.98 (s, 1H) 8.68 (brs, 1H) 9.25 (brs, 1H) 10.76 (s, 1H) 12.53 (s, 1H). MS (ES) [m+H] calc'd for C₁₂H₁₆N₄S, 249. found 249.

To a stirred solution of N-(5-isobutyl-1H-indazol-3-yl)thiourea (compound 29C, 97.4 mg, 0.392 mmol) in ethanol (3 mL) and 1N HCl (1 mL) was added bromoacetaldehyde diethyl acetal (0.097 mL, 0.784 mmol) at room temperature. The mixture was stirred for 2 h at 80° C., dilution with EtOAc, washed with saturated aqueous NaHCO₃, H₂O, and brine, dried (MgSO₄), filtered, and concentrated in vacuo. The crude oil was passed through a short SiO₂ pad (NH-SiO₂, EtOAc), concentrated in vacuo. Purification by recrystallization (EtOAc-diisopropylether) gave 24.9 mg (23%) of the title compound as a colorless solid. ¹H NMR (300 MHz, DMSO-d₆) δ 0.89 (d, 6H, J=6.59 Hz) 1.75-1.97 (m, 1H) 2.53 (d, 2H, J=6.97 Hz) 6.97 (d, 1H, J=3.58 Hz) 7.18 (dd, 1H, J=8.57, 1.41 Hz) 7.31 (d, 1H, J=8.48 Hz) 7.34 (d, 1H, J=3.58 Hz) 7.86 (s, 1H) 11.20 (s, 1H) 12.20 (s, 1H). MS (ES) [m+H] calc'd for C₁₄H₁₆N₄S, 273. found 273.

### Example 30

### N-1,3-Thiazol-2-yl-4-(trifluoromethyl)-1H-indazol-3-amine

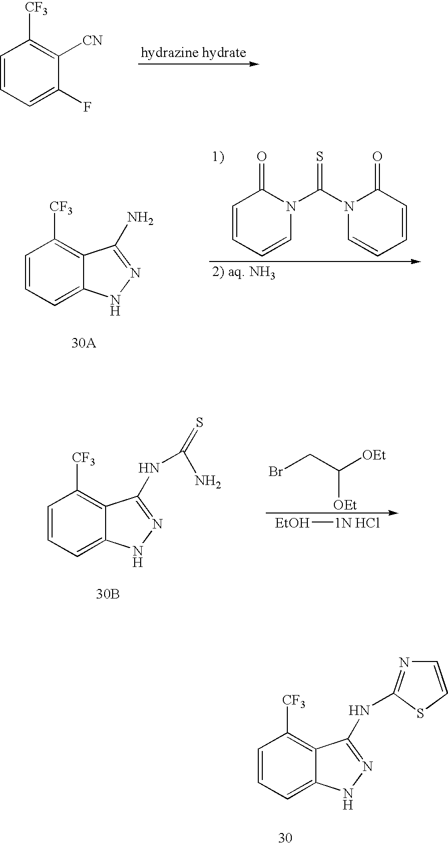

4-(Trifluoromethyl)-1H-indazol-3-amine (compound 30A) was prepared in 67% yield from 2-fluoro-6-(trifluoromethyl)benzonitrile according to a procedure analogous to that outlined in Example 7. ¹H NMR (300 MHz, DMSO-d₆) δ 4.82 (s, 2H) 7.22-7.49 (m, 2H) 7.62 (d, 1H, J=8.33 Hz) 12.24 (brs, 1H). MS (ES) [m+H] calc'd for C₈H₆F₃N₃, 202. found 202.

N-[4-(Trifluoromethyl)-1H-indazol-3-yl]thiourea (compound 30B) was prepared in 69% yield from 4-(trifluoromethyl)-1H-indazol-3-amine according to a procedure analogous to that outlined in Example 9. ¹H NMR (300 MHz, DMSO-d₆) δ 7.26-7.68 (m, 3H) 7.74-7.92 (m, 1H) 8.12 (brs, 1H) 9.02 (brs, 1H) 13.55 (brs, 1H). MS (ES) [m+H] calc'd for C9H7F3N4S, 261. found 261.

The title compound was prepared in 28% yield from N-[4-(trifluoromethyl)-1H-indazol-3-yl]thiourea according to a procedure analogous to that outlined in Example 28. ¹H NMR (300 MHz, DMSO-d₆) δ 6.75 (s, 1H) 7.14 (s, 1H) 7.52 (d, 2H, J=4.90 Hz) 7.81 (s, 1H) 9.07 (brs, 1H) 13.24 (brs, 1H). MS (ES) [m+H] calc'd for C₁₁H₇F₃N₄S, 285. found 285.

### Example 31

### 7-Bromo-5-propyl-N-1,3-thiazol-2-yl-1H-indazol-3-amine

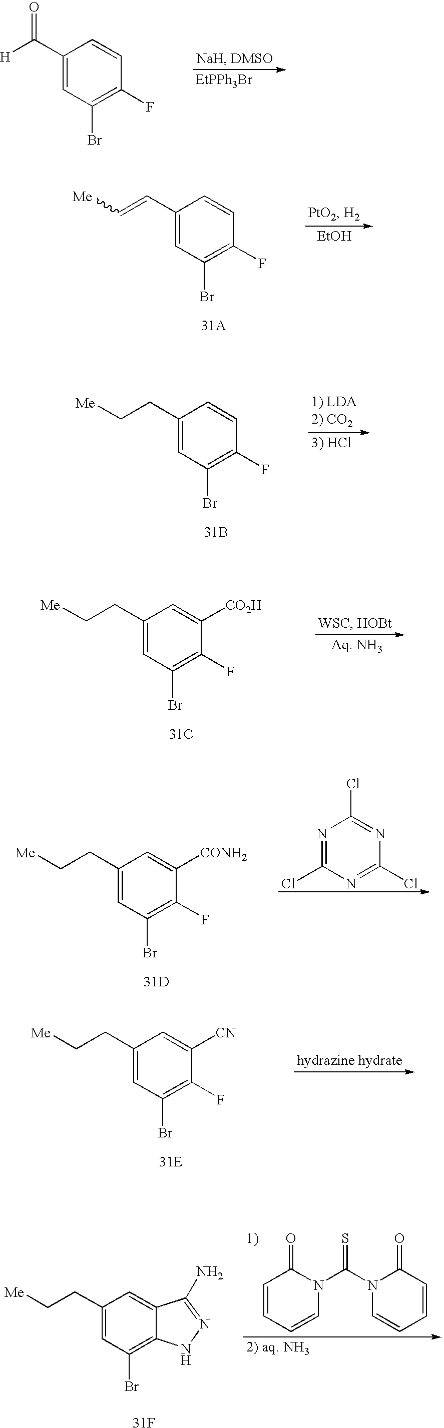

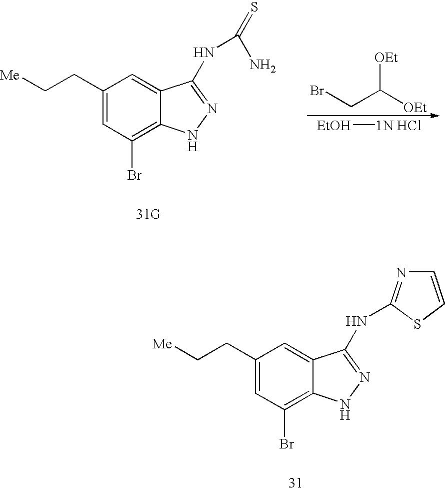

Sodium hydride (60% in oil, 4.32 g, 108 mmol) was added to a solution of 3-bromo-4-fluorobenzaldehyde (10.0 g, 49.3 mmol) in DMSO (100 mL) and THF (200 mL) at room temperature. The whole mixture was stirred at 50° C. for 1.5 h, and then cooled to 0° C. To the mixture was added ethyltriphenylphosphonium bromide (36.6 g, 98.6 mmol) at 0° C. The whole mixture was stirred at room temperature for 30 min. A solution of 3-bromo-4-fluorobenzaldehyde (10.0 g, 49.3 mmol) in DMSO (50 mL) was added to the mixture. The whole mixture was refluxed for 1.5 h, and then cooled to 0° C. Water and 1N HCl (150 mL) were added to the mixture and the mixture was extracted with ethyl acetate. The organic layer was washed with brine, dried (MgSO₄), filtered, and concentrated in vacuo. Purification by silica gel chromatography (hexane) gave 8.35 g of 2-bromo-1-fluoro-4-[(1EZ)-prop-1-en-1-yl]benzene (compound 31A) as a colorless oil which contained mineral oil derived from sodium hydride, and was used for the next step without further purification.

To a stirred solution of the above oil in ethanol (500 mL) was added platinum oxide (200 mg). The mixture was stirred for 2 h under hydrogen atmosphere at room temperature. The catalyst was removed by filtration. The filtrate was concentrated in vacuo to give 2-bromo-1-fluoro-4-propylbenzene (compound 31B) as a colorless oil (6.71 g).

To a stirred solution of lithium diisopropylamide (1.8 M in heptane/THF/ethylbenzene, 3.1 mL, 5.58 mmol) in THF (10 mL) was added a solution of give 2-bromo-1-fluoro-4-propylbenzene (1.0 g, 4.6 mmol) in THF (10 mL) at −78° C. The whole mixture was stirred at room temperature for 1 h. Carbon dioxide was bubbled into the mixture for 1 h, and then the mixture was stirred at room temperature for further 1 h. To the mixture was added 1N HCl, and the mixture was diluted with ethyl acetate. The organic layer was extracted twice with 1N NaOH (50 mL). The aqueous layer was acidified by adding 1N HCl and extracted with ethyl acetate. The organic layer washed with brine, dried (MgSO₄), filtered, and concentrated in vacuo to give 3-bromo-2-fluoro-5-propylbenzoic acid (compound 31C) as colorless oil (400 mg). ¹H NMR (300 MHz, CDCl₃) δ 0.95 (t, 3H, J=7.4 Hz) 1.50-1.70 (m, 2H) 2.50-2.62 (m, 2H) 7.60 (dd, 1H, J=6.0, 2.2 Hz) 7.74 (dd, 1H, J=6.0, 2.2 Hz).

To a stirred solution of 3-bromo-2-fluoro-5-propylbenzoic acid (400 mg, 1.5 mmol), WSC (380 mg, 2.0 mmol), and HOBt (270 mg, 2.0 mmol) in DMF (10 mL) was added aqueous ammonia (28%, 1 mL) at room temperature. The whole mixture was stirred at room temperature for 15 h. Water was added to the mixture and the mixture was extracted with ethyl acetate. The organic layer washed with 1N HCl, saturated aqueous NaHCO₃, and brine, dried (MgSO₄), filtered, and concentrated in vacuo to give 3-bromo-2-fluoro-5-propylbenzamide (compound 31D; 260 mg, 67%) as colorless crystals. The crystals were recrystallized from ethyl acetate-hexane to give colorless prisms. ¹H NMR (300 MHz, CDCl₃) δ 0.93 (t, 3H, J=7.4 Hz) 1.55-1.70 (m, 2H) 2.56-2.61 (m, 2H) 5.86 (brs, 1H) 6.62 (brs, 1H) 7.52 (1H, dd, J=6.5, 2.1 Hz) 7.85 (1H, dd, J=6.5, 2.1 Hz).

To a stirred solution of 3-bromo-2-fluoro-5-propylbenzamide (200 mg, 0.77 mmol), in DMF (5 mL) was added cyanuric chloride (150 mg, 0.84 mmol) at 0° C., and the mixture was stirred at 0° C. for 1 h. To the mixture was added 1N HCl, and the mixture was extracted with ethyl acetate. The organic layer washed with water and brine, dried (MgSO₄), filtered, and concentrated in vacuo to give 3-bromo-2-fluoro-5-propylbenzonitrile (compound 31E; 160 mg, 84%) as a colorless oil. ¹H NMR (300 MHz, CDCl₃) δ 0.95 (t, 3H, J=7.4 Hz) 1.50-1.70 (m, 2H) 2.56-2.61 (m, 2H) 7.36 (dd, 1H, J=5.2, 2.0 Hz) 7.61 (dd, 1H, J=6.4, 2.0 Hz).

7-Bromo-5-propyl-1H-indazol-3-amine (compound 31F) was prepared in 35% yield from 3-bromo-2-fluoro-5-propylbenzonitrile according to a procedure analogous to that outlined in Example 7. ¹H NMR (300 MHz, CDCl₃) δ 0.95 (t, 3H, J=7.38 Hz) 1.59-1.76 (m, 2H) 2.66 (t, 2H, J=7.57 Hz) 4.08 (brs, 2H) 7.28 (s, 1H) 7.36 (s, 1H) 8.87 (brs, 1H). MS (ES) [m+H] calc'd for C₁₀H₁₂BrN₃, 255. found 253, 255.

N-(7-Bromo-5-propyl-1H-indazol-3-yl)thiourea (compound 31G) was prepared in 100% yield from 7-bromo-5-propyl-1H-indazol-3-amine according to a procedure analogous to that outlined in Example 9. ¹H NMR (300 MHz, CDCl₃) δ 0.95 (t, 3H, J=7.35 Hz) 1.60-1.79 (m, 2H) 2.69 (t, 2H, J=7.54 Hz) 6.91 (brs, 1H) 7.42 (s, 1H) 7.48 (d, 1H, J=1.13 Hz) 8.49 (brs, 1H) 9.47 (brs, 2H). MS (ES) [m+H] calc'd for C₁₀H₁₂BrN₃, 314. found 312, 314.

The title compound was prepared in 70% yield from N-(7-bromo-5-propyl-1H-indazol-3-yl)thiourea according to a procedure analogous to that outlined in Example 28. ¹H NMR (300 MHz, DMSO-d₆) δ 0.91 (t, 3H, J=7.35 Hz) 1.55-1.73 (m, 2H) 2.64 (t, 2H, J=7.44 Hz) 7.01 (d, 1H, J=3.58 Hz) 7.36 (d, 1H, J=3.58 Hz) 7.48 (d, 1H, J=1.13 Hz) 7.94 (s, 1H) 11.32 (s, 1H) 12.63 (s, 1H). MS (ES) [m+H] calc'd for C₁₃H₁₃BrN₄S, 338. found 336, 338.

### Example 32

### 4,5-Dimethyl-N-1,3-thiazol-2-yl-1H-indazol-3-amine

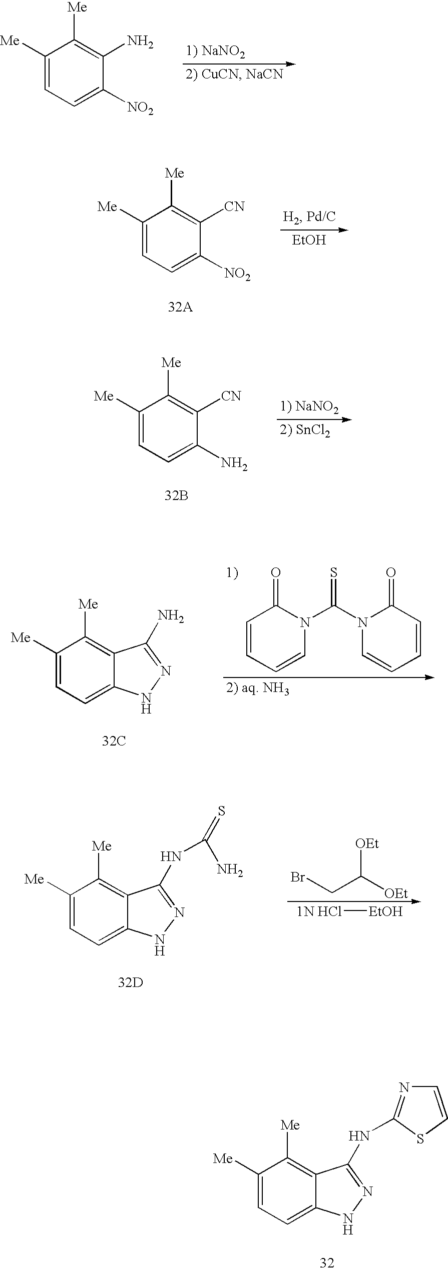

To a stirred mixture of 2,3-dimethyl-6-nitroaniline (10 g, 60 mmol) in conc. HCl (120 mL) was added dropwise a solution of sodium nitrite (4.5 g, 65 mmol) in water (50 mL) at 0° C., and the resulting mixture was stirred for 1 h at room temperature. The reaction mixture was filtered to remove insoluble materials, and the filtrate was cooled to 0° C. To the filtrate was added dropwise a solution of copper cyamide (6.4 g, 72 mmol) and sodium cyamide (8.8 g, 180 mmol) in water (50 mL) at 0° C. The reaction mixture was stirred at room temperature for 18 h and diluted with EtOAc, and the organic layer washed with H₂O and brine, dried (MgSO₄), filtered, and concentrated in vacuo to give 3.3 g (31%) of 2,3-dimethyl-6-nitrobenzonitrile (compound 32A) as a brown solid. ¹H NMR (300 MHz, DMSO-d₆) δ 2.43 (s, 3H) 2.54 (s, 3H) 7.75 (d, 1H, J=8.4 Hz) 8.15 (d, 1H, J=8.4 Hz). MS (ES) [m+H] calc'd for C₉H₈N₂O₂, 177. found 177.

To a stirred solution of 2,3-dimethyl-6-nitrobenzonitrile (3.3 g, 18 mmol) in THF (50 mL) and ethanol (50 mL) was added palladium charcoal (400 mg), and the resulting mixture was stirred for 18 h at room temperature under hydrogen atmosphere. The reaction mixture was filtered to remove the catalyst, and the filtrate was concentrated in vacuo to give 2.5 g (96%) of 6-amino-2,3-dimethyl-benzonitrile (compound 32B) as a yellow solid. ¹H NMR (300 MHz, DMSO-d₆) δ 2.08 (s, 3H) 2.26 (s, 3H) 5.66 (s, 2H) 6.54 (d, 1H, J=8.4 Hz) 7.06 (d, 1H, J=8.4 Hz).

4,5-Dimethyl-1H-indazol-3-amine (compound 32C) was prepared in 46% yield from 6-amino-2,3-dimethyl-benzonitrile according to a procedure analogous to that outlined in Example 28. ¹H NMR (300 MHz, DMSO-d₆) δ 2.23 (s, 3H) 2.49 (s, 3H) 4.83 (s, 2H) 6.92 (d, 1H, J=8.7 Hz) 6.99 (d, 1H, J=8.7 Hz) 11.26 (s, 1H). MS (ES) [m+H] calc'd for C₉H₁₁N₃, 162. found 162.

The title compound was prepared in 73% yield from 4,5-dimethyl-1H-indazol-3-amine according to a procedure analogous to that outlined in Example 28. ¹H NMR (300 MHz, DMSO-d₆) δ 2.28 (s, 3H) 2.50 (s, 3H) 6.76 (s, 1H) 7.14-7.18 (m, 3H) 9.86 (brs, 1H) 12.41 (brs, 1H). MS (ES) [m+H] calc'd for C₁₂H₁₂N₄S, 245. found 245.

### Example 33

### 5-Isopropyl-N-1,3-thiazol-2-yl-1H-indazol-3-amine

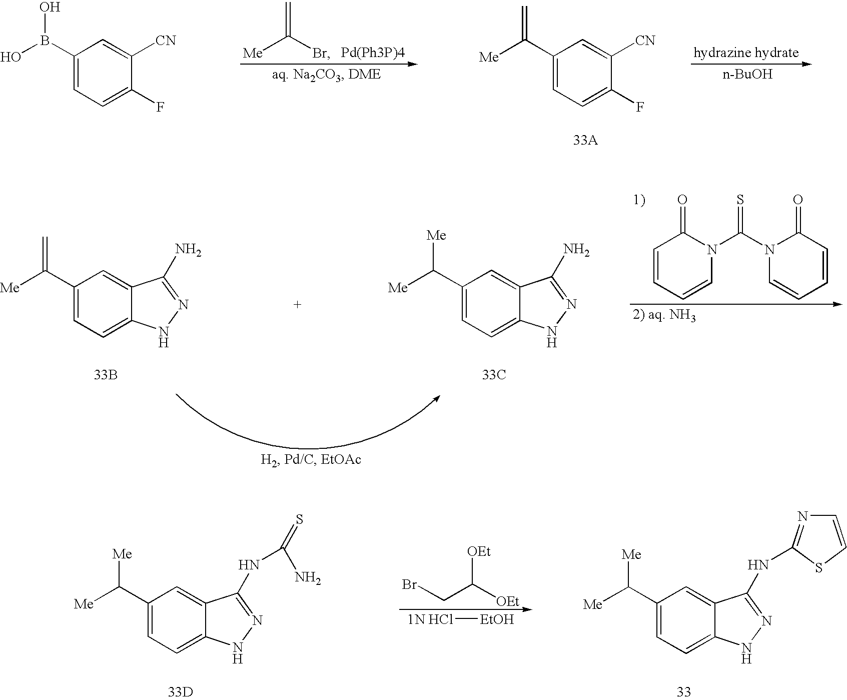

To a stirred solution of 3-cyano-4-fluorophenylboronic acid (1.00 g, 6.1 mmol) in 1,2-dimethoxyethane (30 mL) and 2M sodium carbonate solution (6 mL) were added 2-bromopropene (0.82 mL, 9.15 mmol) and tetrakis(triphenylphosphine)palladium (360 mg, 0.31 mmol) under nitrogen atmosphere at room temperature. The mixture was stirred at 80° C. overnight. After cooling, the mixture was diluted with EtOAc, washed successively with H₂O, 1N HCl, H₂O, and brine, dried (MgSO₄), filtered, and concentrated in vacuo. The precipitate was filtered off and the filtrate was concentrated in vacuo. Purification by silica gel chromatography (EtOAc/hexane=1:30 to 1:10) gave 0.41 g (42%) of 2-fluoro-5-isopropenylbenzonitrile (compound 33A) as a colorless oil. ¹H NMR (300 MHz, CDCl₃) δ 2.13 (s, 3H) 5.19 (s, 1H) 5.36 (s, 1H) 7.04-7.23 (m, 1H) 7.56-7.76 (m, 2H). MS (ES) [m+H] calc'd for C₁₀H₈FN, 162. found 162.

To a stirred solution of 2-fluoro-5-isopropenylbenzonitrile (0.409 g, 2.54 mmol) in n-BuOH (10 mL) was added hydrazine monohydrate (0.37 mL, 7.62 mmol), and the mixture was stirred under reflux overnight. The mixture was concentrated in vacuo. The residue was diluted with EtOAc, washed with H₂O and brine, dried (MgSO₄), filtered, and concentrated in vacuo. The precipitate was collected, and washed with hexane, to give the mixture of 5-allyl-1H-indazol-3-amine (compound 33B) and 5-isopropyl-1H-indazol-3-amine (compound 33C) (2:5). The mixture was dissolved with EtOAc (5 mL). Palladium charcoal (80 mg) was added to the solution, and the mixture was stirred for 2 h under hydrogen atmosphere at room temperature. The catalyst was removed by filtration and the filtrate was concentrated in vacuo to give 170 mg (38% in 2 steps) of compound 33C as a colorless solid. ¹H NMR (300 MHz, DMSO-d₆) δ 1.23 (d, 6H, J=6.97 Hz) 2.86-3.00 (m, 1H) 5.20 (2H, s) 7.13 (d, 2H, J=1.13 Hz) 7.51 (s, 1H) 11.19 (s, 1H). MS (ES) [m+H] calc'd for C₁₀H₃N₃, 176. found 176.

N-(5-Isopropyl-1H-indazol-3-yl)thiourea (compound 33D) was prepared in 100% yield from 5-isopropyl-1H-indazol-3-amine according to a procedure analogous to that outlined in Example 9. ¹H NMR (300 MHz, DMSO-d₆) δ 1.25 (d, 6H, J=6.97 Hz) 2.83-3.08 (m, 1H) 7.23-7.41 (m, 2H) 8.10 (s, 1H) 8.67 (brs, 1H) 9.28 (brs, 1H) 10.78 (brs, 1H) 12.50 (s, 1H). MS (ES) [m+H] calc'd for C₁₁H₁₄N₄S, 235. found 235.

The title compound was prepared in 2.8% yield from N-(5-isopropyl-1H-indazol-3-yl)thiourea according to a a procedure analogous to that outlined in Example 28. ¹H NMR (300 MHz, DMSO-d₆) δ 1.26 (d, 6H, J=6.97 Hz) 2.88-3.05 (m, 1H) 6.96 (d, 1H, J=3.58 Hz) 7.25-7.32 (m, 2H) 7.34 (d, 1H, J=3.58 Hz) 7.98 (s, 1H) 11.20 (brs, 1H) 12.16 (s, 1H). MS (ES) [m+H] calc'd for C₁₃H₁₄N₄S, 259. found 259.

### Example 34

### 5-Isopropoxy-N-1,3-thiazol-2-yl-1H-indazol-3-amine

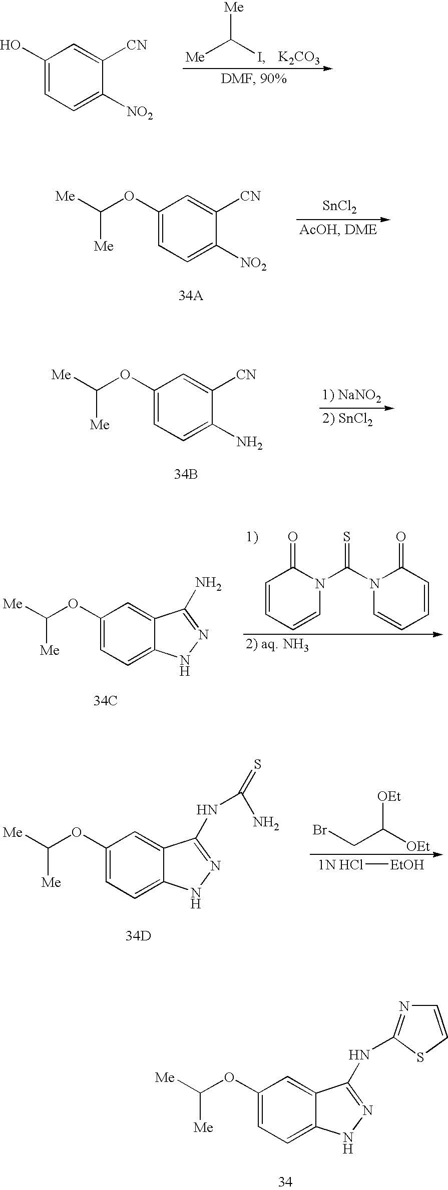

To a stirred solution of 5-hydroxy-2-nitrobenzonitrile (988 mg, 6.0 mmol) in DMF (20 mL) were added potassium carbonate (1.24 g, 4.5 mmol) and isopropyl iodide (0.72 mL, 3.6 mmol) and the mixture was stirred at 80° C. overnight. After dilution with EtOAc, the organic layer washed successively with H₂O, 1N NaOH, 1N HCl, H₂O, and brine, dried (MgSO₄), filtered, and concentrated in vacuo to give 1.11 g (90%) of 5-isopropoxy-2-nitrobenzonitrile (compound 34A) as a light yellow solid. ¹H NMR (300 MHz, CDCl₃) δ 1.42 (d, 6H, J=6.06 Hz) 4.60-4.79 (m, 1H) 7.15 (dd, 1H, J=9.28, 2.84 Hz) 7.28 (d, 1H, J=2.65 Hz) 8.29 (d, 1H, J=9.09 Hz).

To a stirred solution of 5-isopropoxy-2-nitrobenzonitrile (1.31 g, 6.34 mmol) in acetic acid (20 mL) and 1,2-dimethoxyethane (20 mL) was added stannous chloride at room temperature. The mixture was stirred at 60° C. overnight, and concentrated in vacuo. EtOAc and saturated aqueous NaHCO₃ were added to the residue and insoluble material was removed by filtration. The organic layer washed with H₂O and brine, dried (MgSO₄), filtered, and concentrated in vacuo. Purification by silica gel chromatography (NH-SiO₂, hexane:EtOAc=10:1 to 5:1) gave 530 mg (47%) of 2-amino-5-isopropoxybenzonitrile (compound 34B) as a red oil. ¹H NMR (300 MHz, CDCl₃) δ 1.29 (d, 6H, J=6.03 Hz) 4.11 (s, 2H) 4.26-4.50 (m, 1H) 6.68 (d, 1H, J=8.85 Hz) 6.89 (d, 1H, J=2.83 Hz) 6.96 (dd, 1H, J=8.95, 2.92 Hz). MS (ES) [m+H] calc'd for C₁₀H₁₂N₂O, 177. found 177.

To a stirred solution of 2-amino-5-isopropoxybenzonitrile (475 mg, 2.695 mmol) in conc. HCl (10 mL) was added sodium nitrite (224 mg, 3.23 mmol) in H₂O at 0° C., and the mixture was stirred for 30 min. The resulting solution was added dropwise to stannous chloride (1.54 g, 8.08 mmol) in conc. HCl (10 ml). The mixture was stirred for 2 h at room temperature, and concentrated in vacuo. To the residue were added 8N NaOH and EtOAc, and the insoluble material was removed by filtration. The organic layer was separated, washed with H₂O and brine, dried (MgSO₄), filtered, and concentrated in vacuo. The precipitate was collected and washed with diisopropyl ether to give 0.329 g (64%) of 5-isopropoxy-1H-indazol-3-amine (compound 34C) as a colorless solid. ¹H NMR (300 MHz, DMSO-d₆) δ 1.27 (d, 6H, J=6.06 Hz) 4.32-4.62 (m, 1H) 5.13 (s, 2H) 6.86 (dd, 1H, J=8.90, 2.08 Hz) 7.11 (d, 1H, J=9.09 Hz) 7.18 (d, 1H, J=2.27 Hz) 11.12 (s, 1H). MS (ES) [m+H] calc'd for C₁₀H₁₃N₃O, 192. found 192.

N-(5-Isopropoxy-1H-indazol-3-yl)thiourea (compound 34D) was prepared in 43% yield from 5-isopropoxy-1H-indazol-3-amine according to a procedure analogous to that outlined in Example 9. ¹H NMR (300 MHz, DMSO-d₆) δ 1.31 (d, 6H, J=6.03 Hz) 4.39-4.72 (m, 1H) 6.99 (dd, 1H, J=9.04, 2.26 Hz) 7.32 (d, 1H, J=9.04 Hz) 7.73 (d, 1H, J=2.07 Hz) 8.67 (brs, 1H) 9.28 (brs, 1H) 10.71 (s, 1H) 12.45 (s, 1H). MS (ES) [m+H] calc'd for C₁₁H₁₄N₄OS, 251. found 251.

The title compound was prepared in 34% yield from N-(5-isopropoxy-1H-indazol-3-yl)thiourea according to a procedure analogous to that outlined in Example 28. ¹H NMR (300 MHz, DMSO-d₆) δ 1.31 (d, 6H, J=6.03 Hz) 4.37-4.62 (m, 1H) 6.93-7.00 (m, 2H) 7.29 (d, 1H, J=8.85 Hz) 7.34 (d, 1H, J=3.58 Hz) 7.62 (d, 1H, J=2.07 Hz) 11.11 (brs, 1H) 12.11 (s, 1H). MS (ES) [m+H] calc'd for C₁₃H₁₄N₄OS, 275. found 275.

### Example 35

### 5-(2-Methoxy-1-methylethoxy)-N-1,3-thiazol-2-yl-1H-indazol-3-amine

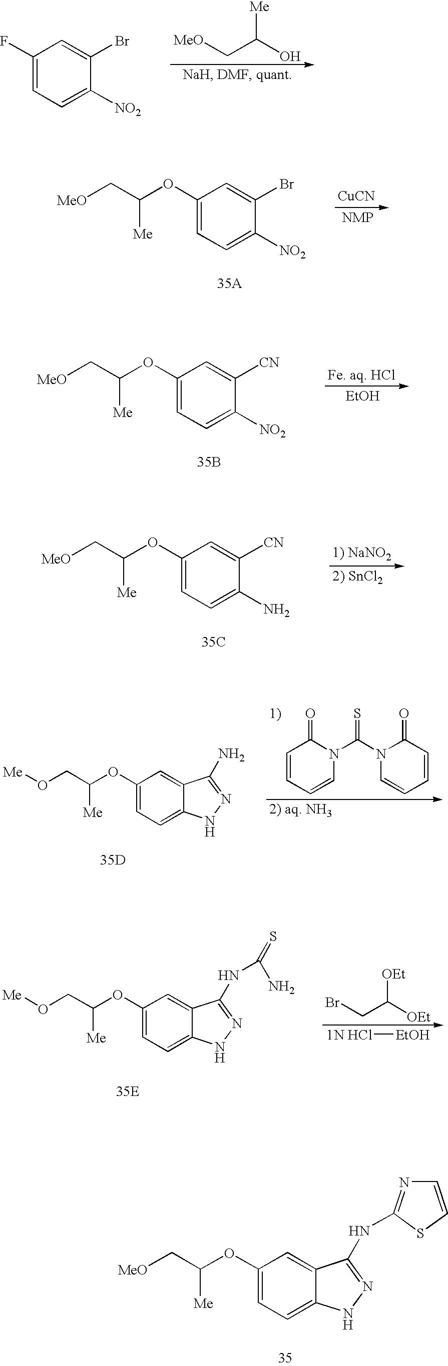

To a stirred suspension of sodium hydride (60% oil dispersion, 1.36 g, 34.1 mmol) in DMF (50 mL) was added 1-methoxy-2-propanol (3.4 mL, 34.1 mmol) at 0° C., and the mixture was stirred for 30 min at 0° C. To the mixture was added a solution of 1-bromo-5-fluoro-2-nitrobenzene (5.0 g, 22.7 mmol) in DMF (10 mL) at 0° C., and the mixture was stirred for 1 h at room temperature. To the mixture was carefully added 1N HCl (50 mL) at 0° C. The aqueous layer was extracted with EtOAc, and the organic layer washed with 1N HCl, H₂O, and brine, dried (MgSO₄), filtered, and concentrated in vacuo to give 6.60 g of 2-bromo-4-(2-methoxy-1-methylethoxy)-1-nitrobenzene (compound 35A) as a light yellow oil, which was used for the next step without further purification. ¹H NMR (300 MHz, CDCl₃) δ 1.34 (d, 3H, J=6.40 Hz) 3.40 (s, 3H) 3.47-3.62 (m, 2H) 4.57-4.71 (m, 1H) 6.94 (dd, 1H, J=9.23, 2.64 Hz) 7.25-7.28 (m, 1H) 7.97 (d, 1H, J=9.23 Hz).

To a stirred solution of 2-bromo-4-(2-methoxy-1-methylethoxy)-1-nitrobenzene (2.007 g, 6.92 mmol) in 1-methyl-2-pyrrolidinone (20 mL) was added copper cyamide (0.75 g, 8.3 mmol) at room temperature. The mixture was stirred for 30 min at 150° C. After cooling, the mixture was diluted with EtOAc. The organic layer washed successively with H₂O, 1N HCl, H₂O, and brine, dried (MgSO₄), filtered, and concentrated in vacuo. Purification by silica gel chromatography (hexane:EtOAc=30:1 to 10:1 to 5:1 to 3:1) gave 1.46 g (89%) of 5-(2-methoxy-1-methylethoxy)-2-nitrobenzonitrile (compound 35B) as a brown oil. ¹H NMR (300 MHz, CDCl₃) δ 1.37 (d, 3H, J=6.40 Hz) 3.39 (s, 3H) 3.49-3.66 (m, 2H) 4.63-4.81 (m, 1H) 7.24 (dd, 1H, J=9.42, 2.83 Hz) 7.37 (d, 1H, J=2.83 Hz) 8.28 (d, 1H, J=9.23 Hz). MS (ES) [m+H] calc'd for C₁₁H₁₂N₂O₄, 237. found 237.

2-Amino-5-(2-methoxy-1-methylethoxy)benzonitrile (compound 35C) was prepared in 49% yield from 5-(2-methoxy-1-methylethoxy)-2-nitrobenzonitrile according to a procedure analogous to that outlined in Example 34. ¹H NMR (300 MHz, CDCl₃) δ 1.26 (d, 3H, J=6.22 Hz) 3.40 (s, 3H) 3.42-3.57 (m, 2H) 4.13 (s, 2H) 4.27-4.40 (m, 1H) 6.68 (d, 1H, J=8.85 Hz) 6.95 (d, 1H, J=2.83 Hz) 6.98-7.05 (m, 1H). MS (ES) [m+H] calc'd for C₁₁H₁₄N₂O₂, 207. found 207.

5-(2-Methoxy-1-methylethoxy)-1H-indazol-3-amine (compound 35C) was prepared in 87% yield from 2-amino-5-(2-methoxy-1-methylethoxy)benzonitrile according to a procedure analogous to that outlined in Example 28. ¹H NMR (300 MHz, DMSO-d₆) δ 1.22 (d, 3H, J=6.22 Hz) 3.30 (s, 3H) 3.40-3.55 (m, 2H) 4.35-4.54 (m, 1H) 5.16 (s, 2H) 6.88 (dd, 1H, J=8.85, 2.45 Hz) 7.12 (d, 1H, J=8.85 Hz) 7.22 (d, 1H, J=2.26 Hz) 11.16 (s, 1H). MS (ES) [m+H] calc'd for C₁₁H₁₅N₃O₂, 222. found 222.

N-[5-(2-Methoxy-1-methylethoxy)-1H-indazol-3-yl]thiourea (compound 35E) was prepared in 68% yield from 5-(2-methoxy-1-methylethoxy)-1H-indazol-3-amine according to a procedure analogous to that outlined in Example 9. ¹H NMR (300 MHz, DMSO-d₆) δ 1.27 (d, 3H, J=6.22 Hz) 3.31 (s, 3H) 3.42-3.60 (m, 2H) 4.40-4.61 (m, 1H) 7.01 (dd, 1H, J=8.95, 2.35 Hz) 7.33 (d, 1H, J=8.85 Hz) 7.77 (d, 1H, J=2.07 Hz) 8.68 (brs, 1H) 9.27 (brs, 1H) 10.70 (s, 1H) 12.48 (s, 1H). MS (ES) [m+H] calc'd for C₁₂H₁₆N₄O₂S, 281. found 281.

The title compound was prepared in 49% yield from N-[5-(2-methoxy-1-methylethoxy)-1H-indazol-3-yl]thiourea according to a procedure analogous to that outlined in Example 28. ¹H NMR (300 MHz, DMSO-d₆) δ 1.27 (d, 3H, J=6.44 Hz) 3.31 (s, 3H) 3.39-3.65 (m, 2H) 4.36-4.59 (m, 1H) 6.96 (d, 1H, J=3.41 Hz) 7.00 (dd, 1H, J=9.09, 2.27 Hz) 7.30 (d, 1H, J=9.09 Hz) 7.34 (d, 1H, J=3.41 Hz) 7.66 (d, 1H, J=1.89 Hz) 11.10 (s, 1H) 12.13 (s, 1H). MS (ES) [m+H] calc'd for C₁₄H₁₆N₄O₂S, 305. found 305.

### Example 36

### 5-Isobutyl-7-methoxy-N-1,3-thiazol-2-yl-1H-indazol-3-amine

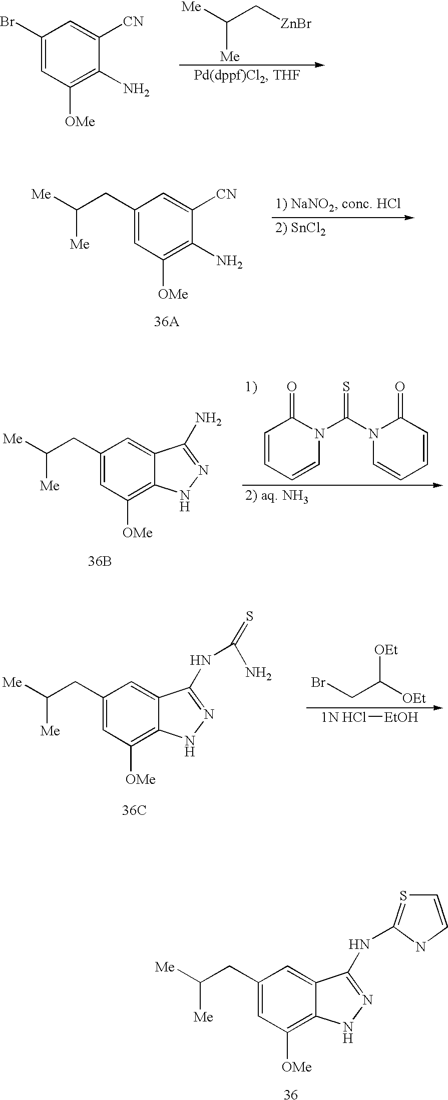

To a stirred solution of 2-amino-5-bromo-3-methoxybenzonitrile (3.41 g, 15 mmol) in THF (60 mL) were added 1,1′-bis(diphenylphosphino)ferrocenedichloro palladium (1.23 g, 1.5 mmol), and 2-methylpropylzinc bromide (0.5 M in THF, 75 mL, 37.5 mmol) at room temperature under nitrogen atmosphere, and the mixture was stirred for 2 h. To the mixture was added H₂O, and the insoluble material was removed by filtration and washed with EtOAc. The organic layer of the filtrate was separated, washed with H₂O and brine, dried (MgSO₄), filtered, and concentrated in vacuo. Purification by silica gel chromatography (hexane:EtOAc=10:1 to 5:1) gave 2.977 g (97%) of 2-amino-5-isobutyl-3-methoxybenzonitrile (compound 36A) as a light yellow oil. ¹H NMR (300 MHz, CDCl₃) δ 0.88 (d, 6H, J=6.82 Hz) 1.69-1.90 (m, 1H) 2.35 (d, 2H, J=7.19 Hz) 3.86 (s, 3H) 4.43 (brs, 2H) 6.68 (d, 1H, J=1.51 Hz) 6.76 (d, 1H, J=1.89 Hz). MS (ES) [m+H] calc'd for C₁₂H₁₆N₂O, 205. found 205.

5-Isobutyl-7-methoxy-1H-indazol-3-amine (compound 36B) was prepared in 94% yield from 2-amino-5-isobutyl-3-methoxybenzonitrile according to a procedure analogous to that outlined in Example 28. ¹H NMR (300 MHz, DMSO-d₆) δ 0.88 (d, 6H, J=6.44 Hz) 1.77-1.95 (m, 1H) 2.46 (d, 2H, J=7.19 Hz) 3.86 (s, 3H) 5.11 (s, 2H) 6.54 (s, 1H) 6.97 (s, 1H) 11.34 (s, 1H). MS (ES) [m+H] calc'd for C₁₂H₁₇N₃O, 220. found 220.

N-(5-Isobutyl-7-methoxy-1H-indazol-3-yl)thiourea (compound 36C) was prepared in 96% yield from 5-isobutyl-7-methoxy-1H-indazol-3-amine according to a procedure analogous to that outlined in Example 9. ¹H NMR (300 MHz, DMSO-d₆) δ 0.90 (d, 6H, J=6.44 Hz) 1.82-1.97 (m, 1H) 2.44-2.52 (m, 2H) 3.92 (s, 3H) 6.70 (s, 1H) 7.52 (s, 1H) 8.63 (brs, 1H) 9.21 (brs, 1H) 10.66 (s, 1H) 12.74 (s, 1H). MS (ES) [m+H] calc'd for C₁₃H₁₈N₄OS, 279. found 279.

The title compound was prepared in 47% yield from N-(5-isobutyl-7-methoxy-1H-indazol-3-yl)thiourea according to a procedure analogous to that outlined in Example 28. ¹H NMR (300 MHz, DMSO-d₆) δ 0.90 (d, 6H, J=6.44 Hz) 1.81-1.97 (m, 1H) 2.49-2.50 (m, 2H) 3.92 (s, 3H) 6.68 (s, 1H) 6.95 (d, 1H, J=3.79 Hz) 7.32 (d, 1H, J=3.79 Hz) 7.41 (s, 1H) 11.08 (brs, 1H) 12.36 (s, 1H). MS (ES) [m+H] calc'd for C₁₅H₁₈N₄OS, 303. found 303.

Biological Testing

The activity of compounds as glucokinase activators may be assayed in vitro, in vivo or in a cell line. Provided below is an enzymatic glucokinase activity assay.

Purified glucokinase may be obtained as follows. DNA encoding residues 12-465 of the full-length sequence of the human enzyme may be amplified by PCR and cloned into the HindIII and EcoRI sites of pFLAG-CTC (Sigma). SEQ. I.D. No. 1 corresponds to residues 13-466 of glucokinase.

The expression of recombinant glucokinase protein may be carried out by transformation and growth of DH10b-T1r E. coli cells incorporating the (pFLAG-CTC) plasmid in LB media. Protein expression can be induced in this system by the addition of IPTG to the culture medium.

Recombinant protein may be isolated from cellular extracts by passage over Sepharose Q Fast Flow resin (Pharmacia). This partially purified GK extract may then be further purified by a second passage over Poros HQ10 (Applied Biosystems). The purity of GK may be determined on denaturing SDS-PAGE gel. Purified GK may then be concentrated to a final concentration of 20.0 mg/ml. After flash freezing in liquid nitrogen, the proteins can be stored at −78° C. in a buffer containing 25 mM TRIS-HCl pH 7.6, 50 mM NaCl, and 0.5 mM TCEP.

It should be noted that a variety of other expression systems and hosts are also suitable for the expression of glucokinase, as would be readily appreciated by one of skill in the art.

The activation properties of compounds for GK may be determined using a black 384-well-plate format under the following reaction conditions: 25 mM Hepes pH 7.2, 25 mM NaCl, 10 mM MgCl₂, 0.01% Brij35, 1 mM DTT, 5 μM ATP, 5 mM Glucose 2% DMSO. The amount of ATP consumed may be determined quantitatively by addition of equal volume of luciferase reagent (luciferase+beetle luciferin—KinaseGlo Luminescent Kinase Assay kit from Promega). The luminescence intensity may be measured by using the Analyst HT from LJL Biosystems.

The assay reaction may be initiated as follows: 4 μl of substrate mixture (12.5 μM ATP and 12.5 mM Glucose) was added to each well of the plate, followed by the addition of 2 μl of activator (2 fold serial dilutions for 11 data points for each activator) containing 10% DMSO. 4 μL of 1.25 nM GK solution may be added to initiate the reaction. The reaction mixture may then be incubated at room temperature for 60 min, and quenched and developed by addition of 10 μL of luciferase reagent. Luminescence intensities of the resulting reaction mixtures may be measured after a 10 min incubation at room temperature. The luminescence intensity may be measured by using the Analyst HT from LJL Biosystems.

pK_{act} and % ACTₘₐₓ values may be calculated by non-linear curve fitting of the compound concentrations and luminescence intensities to a standard inhibition/activation equation. K_{act} is the concentration that displays 50% of the maximal increase in GK activity observed using a saturating activator concentration. % Actₘₐₓ represents the calculated maximal gain in GK enzyme activity at a saturating concentration of the compound.

A 50% solution (5 μL) of the test compound in dimethyl sulfoxide was added to each well of a 384 well black plate (Nalge Nunc). Then, 35 μL of a liquid obtained by diluting GST-hLGK1 obtained in Reference Example 2A with a measurement buffer (50 mM HEPES (pH 7.4), containing 200 mM KCl, 5 mM MgCl₂, 2.5 mM DTT and 50 μM 2′-(or-3′)—O—(N-methylanthraniloyl)adenosine 5′-triphosphate (Mant-ATP) (Jena Bioscience)) was added to each well to 6 μg/mL. Each well was maintained at 37° C. for 10 min, and a 25 mM D-glucose solution (10 μL) was added to start the reaction. After the start of the reaction, each well was allowed to stand at 37° C. for 60 min, before quenching the reaction by adding 25 μL of a reaction quenching solution (200 mM HEPES (pH 7.4), containing 20 mM MgCl₂, 200 mM EDTA, 0.03% Triton-X 100, 0.3% Coating 3 reagent (Caliper Life Sciences)).

Mant-ATP (substrate) and Mant-ADP (reaction resultant product) were separated from each well after quenching the reaction, with a microchip type capillary electrophoresis apparatus 250HTS (Caliper Life Sciences). The reaction rate [(peak height of reaction resultant product)/(peak height of reaction resultant product+peak height of substrate)×100(%)] was calculated from the ratio of the substrate peak height and the reaction resultant product peak height, which were obtained by fluorescence detection (excitation wavelength 355 nm, measurement wavelength 460 nm), and used as an index of the GK activity.

For the control, the reaction rate was calculated in the same manner as above, except that a “50% dimethyl sulfoxide solution” was used instead of the “50% solution of the test compound in dimethyl sulfoxide”.

A percentage obtained by dividing the reaction rate of the well with the test compound (test compound addition group) by the reaction rate of the well with a 50% dimethyl sulfoxide solution alone (control group) was taken as the GK activation value by the test compound, and the concentration of the test compound necessary for the activation of 50% of the maximum activity value determined as an EC₅₀ value. The results are given in Table 1.

TABLE 1
EC₅₀ of Exemplified Compounds against GK

| Compound | EC₅₀ (μM) |
|---|---|
| 7 | 5 |
| 9 | 2.5 |
| 20 | 6.6 |
| 22 | 15 |
| 25 | 8.6 |
| 29 | 1.2 |
| 33 | 2.9 |
| 34 | 1.7 |
| 35 | 2.4 |

It will be apparent to those skilled in the art that various modifications and variations can be made in the compounds, compositions, kits, and methods of the present invention without departing from the spirit or scope of the invention. Thus, it is intended that the present invention cover the modifications and variations of this invention provided they come within the scope of the appended claims and their equivalents.

What is claimed is:

1. A compound comprising: 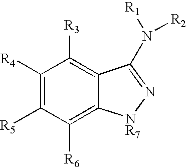
or a polymorph, solvate, ester, tautomer, enantiomer, pharmaceutically acceptable salt or prodrug thereof, wherein
R₁ is hydrogen or a substituent convertible in vivo to hydrogen;
R₂ is a substituted or unsubstituted hetero(C₂₋₁₀)aryl;
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring; and
R₇ is selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or R₇ and R₆ are taken together to form a substituted or unsubstituted ring.

2. The compound according to claim 1, wherein the compound comprises: 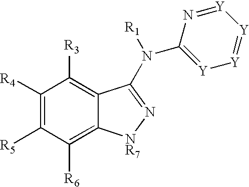
wherein
each Y is independently selected from the group consisting of CR₈ and N; and
R₈ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or two R₈ are taken together to form a substituted or unsubstituted ring.

3. The compound according to claim 2, wherein R₈ is selected from the group consisting of halo, oxycarbonyl, carboxy, carboxamido, acetoxy, (C₁₋₅)alkyl, hydroxy(C₁₋₅)alkyl, aza(C₁₋₅)alkyl and acetylamino(C₁₋₅)alkyl, each substituted or unsubstituted.

4. The compound according to claim 2, wherein R₈ is methyl.

5. The compound according to claim 2, wherein R₈ is —C(O)—O—R₁₃, wherein R₁₃ is selected from the group consisting of a substituted or unsubstituted (C₁₋₁₀)alkyl.

6. The compound according to claim 1, wherein the compound comprises: 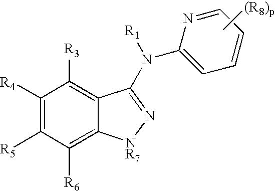
wherein
p is selected from the group consisting of 0, 1, 2, 3 and 4; and
R₈ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or two R₈ are taken together to form a substituted or unsubstituted ring.

7. The compound according to claim 6, wherein R₈ is selected from the group consisting of halo, oxycarbonyl, carboxy, carboxamido, acetoxy, (C₁₋₅)alkyl, hydroxy(C₁₋₅)alkyl, aza(C₁₋₅)alkyl and acetylamino(C₁₋₅)alkyl, each substituted or unsubstituted.

8. The compound according to claim 6, wherein R₈ is methyl.

9. The compound according to claim 6, wherein R₈ is —C(O)—O—R₁₃, wherein R₁₃ is selected from the group consisting of a substituted or unsubstituted (C₁₋₁₀)alkyl.

10. The compound according to claim 1, wherein the compound comprises: 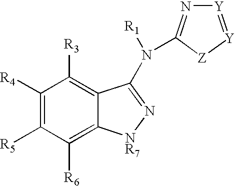
wherein
each Y is independently selected from the group consisting of CR₉ and N;
Z is selected from the group consisting of CR₁₀R₁₁, NR₁₂, S and O;
each R₉ is independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted;
R₁₀ and R₁₁ are each independently selected from the group consisting of halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted; and
R₁₂ is selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted,
or any two of R₉, R₁₀, R₁₁ and R₁₂ are taken together to form a substituted or unsubstituted ring.

11. The compound according to claim 10, wherein R₉ is selected from the group consisting of halo, oxycarbonyl, carboxy, carboxamido, acetoxy, (C₁₋₅)alkyl, hydroxy(C₁₋₅)alkyl, aza(C₁₋₅)alkyl and acetylamino(C₁₋₅)alkyl, each substituted or unsubstituted.

12. The compound according to claim 10, wherein R₉ is methyl.

13. The compound according to claim 10, wherein R₉ is —C(O)—O—R₁₃, wherein R₁₃ is selected from the group consisting of a substituted or unsubstituted (C₁₋₁₀)alkyl.

14. The compound according to claim 10, wherein R₁₀ is selected from the group consisting of halo, oxycarbonyl, carboxy, carboxamido, acetoxy, (C₁₋₅)alkyl, hydroxy(C₁₋₅)alkyl, aza(C₁₋₅)alkyl and acetylamino(C₁₋₅)alkyl, each substituted or unsubstituted.

15. The compound according to claim 10, wherein R₁₀ is methyl.

16. The compound according to claim 10, wherein R₁₀ is —C(O)—O—R₁₃, wherein R₁₃ is selected from the group consisting of a substituted or unsubstituted (C₁₋₁₀)alkyl.

17. The compound according to claim 1, wherein the compound comprises: 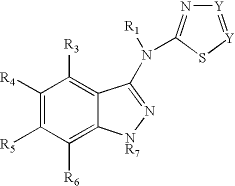
wherein
each Y is independently selected from the group consisting of CR₉ and N; and
each R₉ is independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or two R₉ are taken together to form a substituted or unsubstituted ring.

18. The compound according to claim 1, wherein the compound comprises: 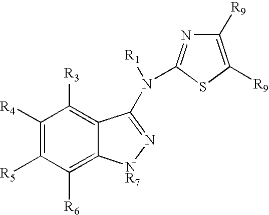
wherein
each R₉ is independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or two R₉ are taken together to form a substituted or unsubstituted ring.

19. The compound according to claim 18, wherein R₉ is selected from the group consisting of halo, oxycarbonyl, carboxy, carboxamido, acetoxy, (C₁₋₅)alkyl, hydroxy(C₁₋₅)alkyl, aza(C₁₋₅)alkyl and acetylamino(C₁₋₅)alkyl, each substituted or unsubstituted.

20. The compound according to claim 18, wherein R₉ is methyl.

21. The compound according to claim 18, wherein R₉ is —C(O)—O—R₁₃, wherein R₁₃ is selected from the group consisting of a substituted or unsubstituted (C₁₋₁₀)alkyl.

22. The compound according to claim 1, wherein R₁ is hydrogen.

23. The compound according to claim 1, wherein R₂ is a substituted or unsubstituted hetero(C₂₋₁₀)aryl comprising a heteroatom at the 2-position.

24. The compound according to claim 1, wherein R₂ is a substituted or unsubstituted hetero(C₂₋₁₀)aryl comprising at least one nitrogen.

25. The compound according to claim 24, wherein the nitrogen is at the 2-position.

26. The compound according to claim 1, wherein R₂ is a substituted or unsubstituted hetero(C₂₋₁₀)aryl comprising an H-bond acceptor.

27. The compound according to claim 26, wherein the H-bond acceptor is at the 2-position.

28. The compound according to claim 1, wherein R₂ is 2-thiazolyl.

29. The compound according to claim 1, wherein R₃ is selected from the group consisting of hydrogen, halo, nitro, oxy, hydroxy, alkoxy, amino and (C₁₋₅)alkyl, each substituted or unsubstituted.

30. The compound according to claim 1, wherein R₃ is selected from the group consisting of hydrogen, (C₁₋₁₅)alkyl and halo(C₁₋₅)alkyl.

31. The compound according to claim 1, wherein R₃ is alkoxy.

32. The compound according to claim 1, wherein R₃ is methoxy.

33. The compound according to claim 1, wherein R₃ is 2-methoxy.

34. The compound according to claim 1, wherein R₄ is selected from the group consisting of hydrogen, halo, nitro, oxy, hydroxy, alkoxy, amino and (C₁₋₅)alkyl, each substituted or unsubstituted.

35. The compound according to claim 1, wherein R₄ is selected from the group consisting of hydrogen, halo, nitro, hydroxy, (C₁₋₅)alkyl, halo(C₁₋₅)alkyl, hydroxy(C₁₋₅)alkyl, (C₁₋₅)alkoxy, (C₁₋₅)alkoxy(C₁₋₅)alkoxy, (C₁₋₅)alkylcarbonyl, amino and (C₁₋₅)alkylcarbonylamino, each substituted or unsubstituted.

36. The compound according to claim 1, wherein R₄ is alkoxy.

37. The compound according to claim 1, wherein R₄ is methoxy.

38. The compound according to claim 1, wherein R₄ is 2-methoxy.

39. The compound according to claim 1, wherein R₄ is —CF₃.

40. The compound according to claim 1, wherein R₅ is selected from the group consisting of hydrogen, halo, nitro, oxy, hydroxy, alkoxy, amino and (C₁₋₅)alkyl, each substituted or unsubstituted.

41. The compound according to claim 1, wherein R₅ is hydrogen.

42. The compound according to claim 1, wherein R₅ is alkoxy.

43. The compound according to claim 1, wherein R₅ is methoxy.

44. The compound according to claim 1, wherein R₅ is 2-methoxy.

45. The compound according to claim 1, wherein R₅ is —CF₃.

46. The compound according to claim 1, wherein R₆ is selected from the group consisting of hydrogen, halo, nitro, oxy, hydroxy, alkoxy, aryloxy, heteroaryloxy, amino, (C₁₋₁₀)alkyl, aryl(C₁₋₁₀)alkyl, (C₄₋₁₂)aryl and hetero(C₂₋₁₀)aryl, each substituted or unsubstituted.

47. The compound according to claim 1, wherein R₆ is selected from the group consisting of hydrogen, halo, (C₁₋₅)alkoxy, (C₁₋₅)alkyl and halo(C₁₋₅)alkyl.

48. The compound according to claim 1, wherein R₆ is alkoxy.

49. The compound according to claim 1, wherein R₆ is methoxy.

50. The compound according to claim 1, wherein R₆ is 2-methoxy.

51. The compound according to claim 1, wherein R₇ is selected from the group consisting of hydrogen and substituted or unsubstituted (C₁₋₅)alkyl.

52. The compound according to claim 1, wherein R₇ is selected from the group consisting of hydrogen, (C₁₋₅)alkyl, aza(C₁₋₅)alkyl, (mono- or di-(C₁₋₅)alkylamino)(C₁₋₅)alkyl and (C₁₋₅)alkoxycarbonylamino(C₁₋₅)alkyl, each substituted or unsubstituted.

53. The compound according to claim 1, wherein
R₁ is hydrogen;
R₂ is 2-thiazolyl;
R₃ is hydrogen, (C₁₋₅)alkyl or halo-(C₁₋₅)alkyl;
R₄ is hydrogen, halo, nitro, hydroxy, (C₁₋₅)alkyl, halo(C₁₋₅)alkyl, hydroxy(C₁₋₅)alkyl, (C₁₋₅)alkoxy, (C₁₋₅)alkoxy(C₁₋₅)alkoxy, (C₁₋₅)alkylcarbonyl, amino or (C₁₋₅)alkylcarbonylamino;
R₅ is hydrogen;
R₆ is hydrogen, halo, (C₁₋₅)alkoxy, (C₁₋₅)alkyl or halo(C₁₋₅)alkyl; and
R₇ is hydrogen, (C₁₋₅)alkyl, aza(C₁₋₅)alkyl, (mono- or di-(C₁₋₅)alkylamino)(C₁₋₅)alkyl or (C₁₋₅)alkoxycarbonylamino(C₁₋₅)alkyl.

54. The compound according to claim 1 selected from the group consisting of:
thiazol-2-yl-(5-trifluoromethyl-1H-indazol-3-yl)-amine;
(4-phenyl-thiazol-2-yl)-(5-trifluoromethyl-1H-indazol-3-yl)-amine;
2-(5-Trifluoromethyl-1H-indazol-3-ylamino)-thiazole-4-carboxylic acid ethyl ester;
(4-phenyl-thiazol-2-yl)-(6-trifluoromethyl-1H-indazol-3-yl)-amine;
4-methyl-N-(5-(trifluoromethyl)-1H-indazol-3-yl)thiazol-2-amine;
N-(1-benzyl-5-trifluoromethyl-1H-indazol-3-yl)-thiazol-2-yl-amine;
5-Bromo-N-1,3-thiazol-2-yl-1H-indazol-3-amine;
5-Chloro-N-1,3-thiazol-2-yl-1H-indazol-3-amine;
1-Methyl-N-1,3-thiazol-2-yl-5-(trifluoromethyl)-1H-indazol-3-amine;
1-Ethyl-N-1,3-thiazol-2-yl-5-(trifluoromethyl)-1H-indazol-3-amine;
1-Isobutyl-N-1,3-thiazol-2-yl-5-(trifluoromethyl)-1H-indazol-3-amine;
tert-Butyl {3-[3-(1,3-thiazol-2-ylamino)-5-(trifluoromethyl)-1H-indazol-1-yl]propyl}carbamate;
1-(3-Aminopropyl)-N-1,3-thiazol-2-yl-5-(trifluoromethyl)-1H-indazol-3-amine;
1-[3-(Dimethylamino)propyl]-N-1,3-thiazol-2-yl-5-(trifluoromethyl)-1H-indazol-3-amine;
tert-Butyl {4-[3-(1,3-thiazol-2-ylamino)-5-(trifluoromethyl)-1H-indazol-1-yl]butyl}carbamate;
1-(4-Aminobutyl)-N-1,3-thiazol-2-yl-5-(trifluoromethyl)-1H-indazol-3-amine;
1-[4-(Dimethylamino)butyl]-N-1,3-thiazol-2-yl-5-(trifluoromethyl)-1H-indazol-3-amine;
[3-(1,3-Thiazol-2-ylamino)-1H-indazol-5-yl]methanol;
5-Ethyl-N-1,3-thiazol-2-yl-1H-indazol-3-amine;
5-Nitro-N-1,3-thiazol-2-yl-1H-indazol-3-amine;
N³-1,3-Thiazol-2-yl-1H-indazole-3,5-diamine;
N-[3-(1,3-Thiazol-2-ylamino)-1H-indazol-5-yl]acetamide;
N-1,3-Thiazol-2-yl-7-(trifluoromethyl)-1H-indazol-3-amine;
7-Fluoro-N-1,3-thiazol-2-yl-1H-indazol-3-amine;
1-[3-(1,3-Thiazol-2-ylamino)-1H-indazol-5-yl]ethanone;
5-Methoxy-N-1,3-thiazol-2-yl-1H-indazol-3-amine;
5-Propyl-N-1,3-thiazol-2-yl-1H-indazol-3-amine;
3-(1,3-Thiazol-2-ylamino)-1H-indazol-5-ol;
5-Isobutyl-N-1,3-thiazol-2-yl-1H-indazol-3-amine;
N-1,3-Thiazol-2-yl-4-(trifluoromethyl)-1H-indazol-3-amine;
7-Bromo-5-propyl-N-1,3-thiazol-2-yl-1H-indazol-3-amine;
4,5-Dimethyl-N-1,3-thiazol-2-yl-1H-indazol-3-amine;
5-Isopropyl-N-1,3-thiazol-2-yl-1H-indazol-3-amine;
5-Isopropoxy-N-1,3-thiazol-2-yl-1H-indazol-3-amine;
5-(2-Methoxy-1-methylethoxy)-N-1,3-thiazol-2-yl-1H-indazol-3-amine; and
5-Isobutyl-7-methoxy-N-1,3-thiazol-2-yl-1H-indazol-3-amine.

55. The compound according to claim 1 selected from the group consisting of:
5-bromo-N-1,3-thiazol-2-yl-1H-indazol-3-amine;
1-methyl-N-1,3-thiazol-2-yl-5-(trifluoromethyl)-1H-indazol-3-amine;
5-nitro-N-1,3-thiazol-2-yl-1H-indazol-3-amine;
N-[3-(1,3-thiazol-2-ylamino)-1H-indazol-5-yl]acetamide;
1-[3-(1,3-thiazol-2-ylamino)-1H-indazol-5-yl]ethanone;
5-isobutyl-N-1,3-thiazol-2-yl-1H-indazol-3-amine;
5-isopropyl-N-1,3-thiazol-2-yl-1H-indazol-3-amine;
5-isopropoxy-N-1,3-thiazol-2-yl-1H-indazol-3-amine; and
5-(2-methoxy-1-methylethoxy)-N-1,3-thiazol-2-yl-1H-indazol-3-amine.

56. The compound according to claim 1, wherein the compound is in the form of a pharmaceutically acceptable salt.

57. A process comprising the steps of
reacting a compound comprising the formula 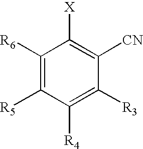
with a compound comprising the formula
H₂NNHR₇
under conditions that form a first reaction product comprising the formula 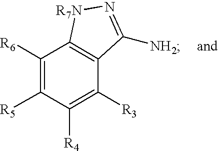
reacting the first reaction product with a compound comprising the formula
X—R₂
under conditions that form a product comprising the formula 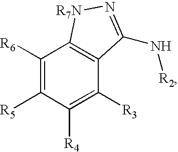
wherein
X is selected from the group consisting of F, Br, Cl and I;
R₂ is a substituted or unsubstituted hetero(C₂₋₁₀)aryl;
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring; and
R₇ is selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or R₇ and R₆ are taken together to form a substituted or unsubstituted ring.

58. A process comprising the steps of
reacting a compound comprising the formula 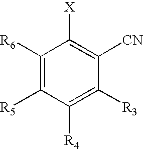
with an alcohol under conditions that form a first reaction product comprising the formula 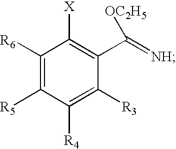
reacting the first reaction product with a compound comprising the formula
NH₂R₁
under conditions that form a second reaction product comprising the formula 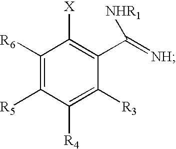
reacting the second reaction product with a compound comprising the formula
H₂NNHR₇
under conditions that form a third reaction product comprising the formula 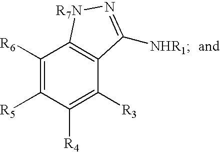
reacting the third reaction product with a compound comprising the formula
X—R₂
under conditions that form a product comprising the formula 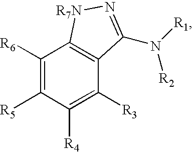
wherein
each X is independently selected from the group consisting of F, Br, Cl and I;
R₁ is hydrogen or a substituent convertible in vivo to hydrogen;
R₂ is a substituted or unsubstituted hetero(C₂₋₁₀)aryl;
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring; and
R₇ is selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or R₇ and R₆ are taken together to form a substituted or unsubstituted ring.

59. A process comprising the steps of
reacting a compound comprising the formula 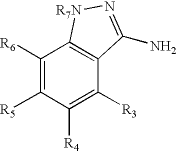
with a compound comprising the formula 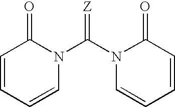
under conditions that form a first reaction product comprising the formula 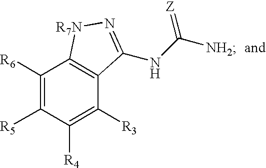
reacting the first reaction product with a compound comprising the formula 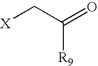
under conditions that form a product comprising the formula 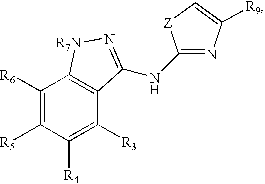
wherein
X is selected from the group consisting of F, Br, Cl and I;
Z is selected from the group consisting of CR₁₀R₁₁, NR₁₂, S and O;
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring;
R₇ is selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or R₇ and R₆ are taken together to form a substituted or unsubstituted ring;
R₉ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted;
R₁₀ and R₁₁ are each independently selected from the group consisting of halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted; and
R₁₂ is selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted,

60. A process comprising the steps of
treating a compound comprising the formula 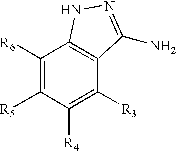
under conditions that form a first reaction product comprising the formula 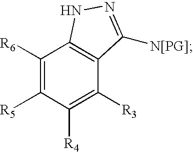
reacting the first reaction product with a compound comprising the formula
X—R₇
under conditions that form a second reaction product comprising the formula 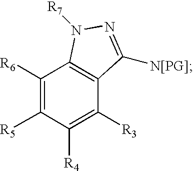
treating the second reaction product under conditions that form a third reaction product comprising the formula 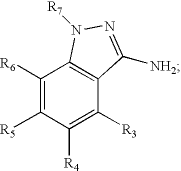
reacting the third reaction product with a compound comprising the formula 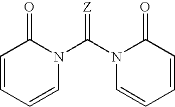
under conditions that form a fourth reaction product comprising the formula 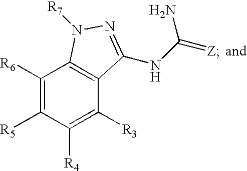
reacting the fourth reaction product with a compound comprising the formula 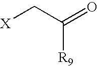
under conditions that form a product comprising the formula 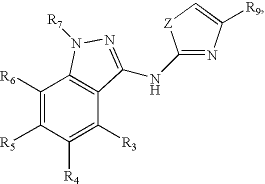
wherein
each X is independently selected from the group consisting of F, Br, Cl and I;
Z is selected from the group consisting of CR₁₀R₁₁, NR₁₂, S and O;
PG is a protecting group;
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring;
R₇ is selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or R₇ and R₆ are taken together to form a substituted or unsubstituted ring;
each R₉ is independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted;
R₁₀ and R₁₁ are each independently selected from the group consisting of halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted; and
R₁₂ is selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted.

61. A process comprising the steps of
treating a compound comprising the formula 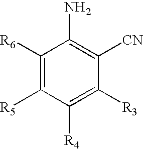
under conditions that form a first reaction product comprising the formula 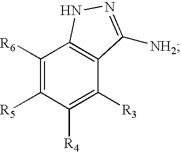
treating the first reaction product under conditions that form a second reaction product comprising the formula 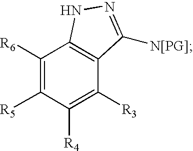
reacting the second reaction product with a compound comprising the formula
X—R₇
under conditions that form a third reaction product comprising the formula 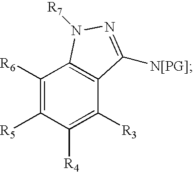
treating the third reaction product under conditions that form a fourth reaction product comprising the formula 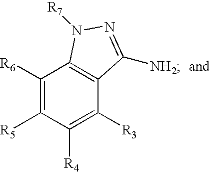
reacting the fourth reaction product with a compound comprising the formula
X—R₂
under conditions that form a product comprising the formula 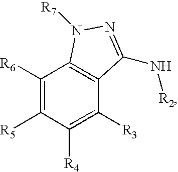
wherein
each X is independently selected from the group consisting of F, Br, Cl and I;
PG is a protecting group;
R₂ is a substituted or unsubstituted hetero(C₂₋₁₀)aryl;
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring; and
R₇ is selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or R₇ and R₆ are taken together to form a substituted or unsubstituted ring.

62. A process comprising the steps of
reacting a compound comprising the formula 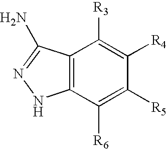
with 2,5-hexandione under conditions that form a first reaction product comprising the formula 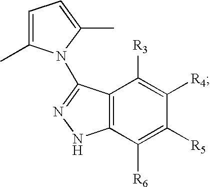
reacting the first reaction product with a compound comprising the formula
X—R₇
under conditions that form a second reaction product comprising the formula 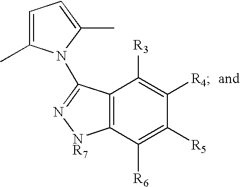
treating the second reaction product under conditions that form a product comprising the formula 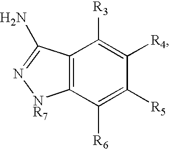
wherein
X is selected from the group consisting of F, Br, Cl and I;
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring; and
R₇ is selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or R₇ and R₆ are taken together to form a substituted or unsubstituted ring.

63. A process comprising the steps of
reacting a compound comprising the formula 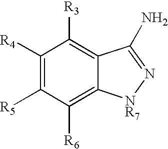
with NH₄SCN under conditions that form a first reaction product comprising the formula 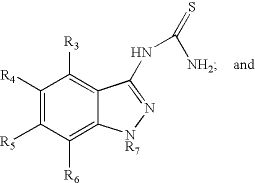
treating the first reaction product under conditions that form a product comprising the formula 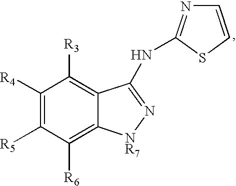
wherein
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring; and
R₇ is selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or R₇ and R₆ are taken together to form a substituted or unsubstituted ring.

64. A process comprising the steps of
treating a compound comprising the formula 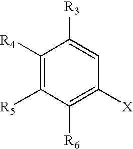
under conditions that form a first reaction product comprising the formula 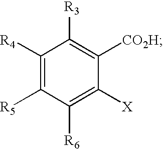
treating the first reaction product under conditions that form a second reaction product comprising the formula 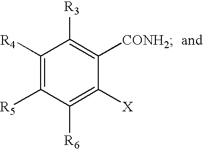
reacting the second reaction product with 2,4,6-trichloro-1,3,5-triazine under conditions that form a product comprising the formula 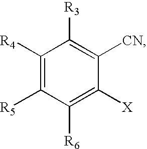
wherein
X is selected from the group consisting of F, Br, Cl and I; and
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring.

65. A process comprising the steps of
treating a compound comprising the formula 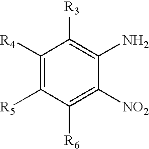
under conditions that form a first reaction product comprising the formula 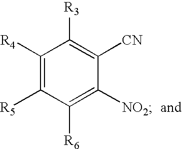
treating the first reaction product under conditions that form a product comprising the formula 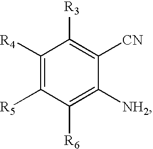
wherein
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring.

66. A compound comprising: 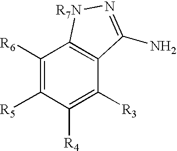
wherein
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring; and
R₇ is selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or R₇ and R₆ are taken together to form a substituted or unsubstituted ring.

67. A compound comprising: 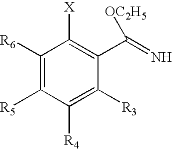
wherein
X is selected from the group consisting of F, Br, Cl and I; and
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring.

68. A compound comprising: 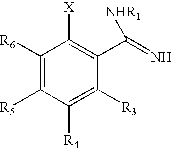
wherein
X is selected from the group consisting of F, Br, Cl and I;
R₁ is hydrogen or a substituent convertible in vivo to hydrogen; and
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring.

69. A compound comprising: 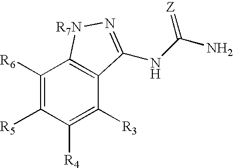
wherein
Z is selected from the group consisting of CR₁₀R₁₁, NR₁₂, S and O;
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring;
R₇ is selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or R₇ and R₆ are taken together to form a substituted or unsubstituted ring;
R₁₀ and R₁₁ are each independently selected from the group consisting of halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted; and
R₁₂ is selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted.

70. A compound comprising: 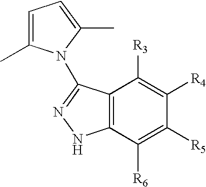
wherein
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring.

71. A compound comprising: 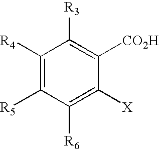
wherein
X is selected from the group consisting of F, Br, Cl and I; and
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring.

72. A compound comprising: 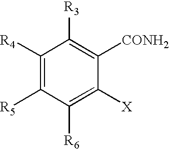
wherein
X is selected from the group consisting of F, Br, Cl and I; and
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring.

73. A compound comprising: 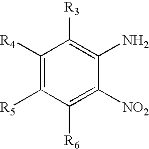
wherein
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring.

74. A compound comprising: 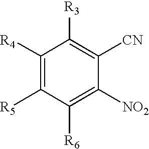
wherein
R₃, R₄, R₅ and R₆ are each independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, aza(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₂₋₁₀)aryl, (C₉₋₁₂)bicycloaryl and hetero(C₄₋₁₂)bicycloaryl, each substituted or unsubstituted, or any two of R₃, R₄, R₅ and R₆ are taken together to form a substituted or unsubstituted ring.

75. A pharmaceutical composition comprising as an active ingredient a compound according to claim 1.

76. A pharmaceutical composition comprising a compound according to claim 1, wherein the composition is adapted for administration by a route selected from the group consisting of orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, and intrathecally.

77. A kit comprising:
a compound according to claim 1; and
instructions which comprise one or more forms of information selected from the group consisting of indicating a disease state for which the compound is to be administered, storage information for the compound, dosing information and instructions regarding how to administer the compound.

78. An article of manufacture comprising:
a compound according to claim 1; and
packaging materials.

79. A therapeutic method comprising administering a compound according to claim 1 to a subject.

80. A method of activating glucokinase comprising contacting glucokinase with a compound according to claim 1.

81. A method of activating glucokinase comprising causing a compound according to claim 1 to be present in a subject in order to activate glucokinase in vivo.

82. A method of activating glucokinase comprising administering a first compound to a subject that is converted in vivo to a second compound wherein the second compound activates glucokinase in vivo, the second compound being a compound according to claim 1.

83. A method of treating a disease state for which increasing glucokinase activity ameliorates the pathology and/or symptomology of the disease state, the method comprising causing a compound according to claim 1 to be present in a subject in a therapeutically effective amount for the disease state.

84. The method according to claim 83, wherein the disease state is selected from the group consisting of hyperglycemia, diabetes, dyslipidaemia, obesity, insulin resistance, metabolic syndrome X, impaired glucose tolerance, polycystic ovary syndrome and cardiovascular disease.

85. A method of treating a disease state for which increasing glucokinase activity ameliorates the pathology and/or symptomology of the disease state, the method comprising administering a compound according to claim 1 to a subject, wherein the compound is present in the subject in a therapeutically effective amount for the disease state.

86. The method according to claim 85, wherein the disease state is selected from the group consisting of hyperglycemia, diabetes, dyslipidaemia, obesity, insulin resistance, metabolic syndrome X, impaired glucose tolerance, polycystic ovary syndrome and cardiovascular disease.

87. A method of treating a disease state for which increasing glucokinase activity ameliorates the pathology and/or symptomology of the disease state, the method comprising administering a first compound to a subject that is converted in vivo to a second compound wherein the second compound activates glucokinase in vivo, the second compound being a compound according to claim 1.

88. The method according to claim 87, wherein the disease state is selected from the group consisting of hyperglycemia, diabetes, dyslipidaemia, obesity, insulin resistance, metabolic syndrome X, impaired glucose tolerance, polycystic ovary syndrome and cardiovascular disease.
